(19) United States
(12) Patent Application Publication
(10) Pub. No.: US 2026/0021110 A1
(43) Pub. Date: Jan. 22, 2026

(54) METHODS OF ORAL CONTRACEPTION BY CONTROLLED RELEASE OF DIENOGEST AND ETHYNYL ESTRADIOL

(71) Applicant: CHEMO RESEARCH, S.L., Madrid (ES)

(72) Inventor: Enrico Colli, Madrid (ES)

(21) Appl. No.: 18/777,376

(22) Filed: Jul. 18, 2024

(51) Int. Cl. A61K 31/569; A61K 31/567; A61P 15/18

(52) U.S. Cl. CPC A61K 31/569; A61K 31/567; A61P 15/18

(57) ABSTRACT

Methods for oral contraception comprising administering an oral contraceptive composition comprising 17α-cyanomethyl-17-β-hydroxyestra-4,9-dien-3-one (dienogest, DNG) and 17α-ethinylestradiol (ethynyl estradiol, EE) are provided. Embodiments of the disclosed methods provide effective contraception with a Pearl Index of less than 0.7 when up to one or two daily oral doses are skipped or delayed.

### FIELD

Embodiments of the present invention pertains to the field of women's health and more specifically contraception. In particular, it relates to methods for oral contraception using oral contraceptive compositions comprising 17α-cyanomethyl-17-β-hydroxyestra-4,9-dien-3-one (dienogest) (DNG) and 17α-ethinylestradiol (ethynyl estradiol) (EE). Methods of the present disclosure can achieve surprisingly high levels of contraception efficacy, as well as unusually good bleeding profiles relative to known contraceptives.

### BACKGROUND

Combined oral contraceptives (COCs) are among the most common methods of reversible birth control. Although effective in preventing pregnancy, these combinations have shown some safety and tolerability issues.

It is known that some women experience adverse events while taking oral contraceptives, including breast tenderness, headache, nausea, mood swings and loss of libido. In addition, epidemiological studies have shown a dose dependent increased risk of arterial and venous thrombotic events (VTEs). Presence of adverse events, as well as irregular bleeding patterns are often leading to lack of compliance and discontinuation, exposing women to a higher risk of pregnancy. The use of low-dose oral contraceptives containing ≤35 mcg EE has been reported to decrease incidence of VTEs and other adverse events of COCs (Helmerhorst F M, 1998; Meade T W. 1982; Gerstman B B et al., 1991; Lidegaard O. 1993; Guida et al. 2010; Stanczyk et al. 2013). Accordingly, there has been a sustained effort to reduce the dose of EE in COCs.

The synthetic progestogen dienogest (DNG) is known to have a protective effect on the endometrium, to be well tolerated and associated with high contraceptive efficacy when combined with the synthetic estrogen ethinyl estradiol (EE). The COC Valette™ (Bayer Schering Pharma AG, Germany), containing 2 mg DNG and 0.03 mg EE, is a low dose DNG/EE oral contraceptive marketed with a regimen of 21 days active pill intake followed by 7 tablet-free days. This COC has also been studied in an extended regimen of 84 days of pill intake followed by 7 tablet-free days. The extended-cycle use was reported to be effective and mostly well tolerated, appearing to be a favorable option for women who don't wish to bleed every month (Wiegratz et al. 2011). Wiegratz et al. 2003 evaluated the effect of decreasing the EE dose of a DNG/EE contraceptive combination to 20 mcg EE or below. In particular, they compared the contraceptive activity and cycle control of 30 mcg EE/2 mg DNG with oral dosage forms comprising 20 mcg EE+2 mg DNG (20 EE/DNG), 10 mcg EE+2 mg estradiol valerate (EV)+2 mg DNG (EE/EV/DNG) or 20 mcg EE+100 mcg levonorgestrel (LNG) (EE/LNG). The tablets were taken for six cycles (21 days and 7 days of hormone-free interval). In this study, the cycle control was significantly better with 30 EE/DNG or EE/LNG than with 20 EE/DNG or EE/EV/DNG. Thus, the reduction of the EE dose to 20 mcg or lower had to be compensated by more potent progestins, in order to maintain an appropriate cycle control.

This is in line with the findings of Stanczyk et al. 2013, showing that while lowering the dose of EE from 35 to 20 mcg resulted in reduced symptoms of breast tenderness, nausea and dizziness, it was found to be associated with a higher incidence of unscheduled bleeding than in COCs with a higher EE dose.

CA 02594939 (WO 2006/087177) relates to an oral pharmaceutical form comprising DNG equal to or less than 2.0 mg and EE less than 0.030 mg, where DNG is released proportionately in two phases and one of the phases is released with a time delay relative to the other phase. More specifically, DNG has a rapid (non-slow) in vitro release within the first phase and a delayed (slow) in vitro release within the second phase; EE has a conventional rapid in vitro release. This document does neither provide any experimental data showing the efficacy of the disclosed compositions in the inhibition of ovulation nor information on its bleeding pattern.

Besides, Guida et al. 2010 evaluated an oral contraceptive with a four-phasic dose regimen wherein the progestogen is DNG and EE is replaced by natural 17β-estradiol (E2), in the form of estradiol valerate (EV). This oral contraceptive includes four hormonal dosage steps in which estrogen and progestin doses follow as close as possible the menstrual cycle physiology. In particular, E2V is combined with DNG in a four-phasic dose regimen wherein the first two tablets contain 3 mg E2V; the next five tablets include 2 mg E2V+2 mg DNG, followed by 17 tablets with 2 mg E2V+3 mg DNG; followed by two tablets with 1 mg E2V only, and finally two placebo tablets. Previous attempts to replace EE with E2, particularly when E2 was administered as part of a monophasic or a biphasic treatment resulted in an unacceptable bleeding pattern. The solution provided by Guida et al. 2010 results in a significant increase in the total amount of DNG in comparison to one cycle administration (21+7 hormone-free) administration of Valette™ (61 mg vs 42 mg).

Despite significant progresses, it is still desirable to develop new methods of contraception using DNG/EE oral contraceptive forms containing low dosages of EE, such as 0.02 mg of EE or less, which are well tolerated, provide a reliable contraceptive efficacy and an acceptable bleeding pattern.

### SUMMARY

Embodiments of the present invention relates to methods for oral contraception using an oral contraceptive composition comprises 2 mg of 17α-cyanomethyl-17-β-hydroxyestra-4,9-dien-3-one (dienogest, DNG) and 0.02 mg of 17α-ethinylestradiol (ethynyl estradiol, EE), in an extended-release matrix such that the DNG and the EE are both subject to controlled release.

While low doses of EE (≤0.02 mg per day) in oral contraceptives are associated with increased bleeding (Stanczyk et al. 2024), the prolonged release formulations of the present disclosure in contrast have been shown by the inventors in Phase II clinical trials to be well tolerated (comparable to the immediate release reference product Valette™) and to provide a reliable contraceptive efficacy with an adequate bleeding pattern. For example, prolonged release formulations of the present disclosure exhibit a better profile compared to an immediate-release composition comprising drospirenone (DRSP) 3 mg/EE 0.02 mg, which caused a mean of 3.2-6.2 days bleeding per cycle in two trials of 12 cycles in greater than 1500 participants.

In Example 3 it was shown that all tested DNG and EE prolonged-release formulations (i.e. T1: 2 mg DNG+0.02 mg EE; T2: 1 mg DNG+0.01 mg EE; T3: 2 mg DNG+0.01 mg EE) were safe and well tolerated after multiple dose of once daily administration for 7 days in healthy premenopausal females. The tolerability of the three investigational prolonged release (PR) formulations was comparable to the immediate release reference product Valette™ (2 mg DNG+0.03 mg EE).

After repeated daily intake over 7 days of administration, the main pharmacokinetic characteristics of the three Test PR were:
Tmax shifted from 1.5 hours to approximately 4 hours in the PR formulations.
AUC0-24 h was strictly proportional compared to the immediate release reference product.
Peak Through Fluctuations were reduced in the PR formulations compared to immediate release reference product (Cmax was reduced by approx. 25% and Cmin was slightly higher in the PR formulations).

The Phase II study described in Examples 5 and 8 was performed to assess the inhibition of ovarian activity of the prolonged release DNG and EE formulation described herein, explore its further effects on reproductive parameters and assess the safety and tolerability of three different strengths. The three strengths tested were T1 (EE/DNG 10 μg/1 mg), T2 (10 μg/2 mg), and T3 (20 μg/2 mg). The extended-regimen Velmari® (EE 20 μg/drospirenone 3 mg) was used as a reference product since in the tested drugs the maximum content of EE is of 0.02 mg (which is lower than the 0.03 mg in Valette™).

In this Phase II study, the selected dosage regimen was an extended regimen wherein the oral contraceptives were administered for 4 consecutive cycles (TC1 to TC4), wherein TC1 to TC3 corresponds to a first treatment period (extended) 87 days intake+4 hormone-free days (91); and TC4 to a second treatment period of 24 days intake+4 hormone-free days.

The PR composition at EE 20 μg/DNG 2 mg inhibited the ovarian activity most effectively. It induced 100% inhibition of ovulation, and with no or minimum ovarian activity in the largest number of subjects (using the Hoogland score). The results were highly similar to the reference product Velmari®. The ovarian suppression was confirmed by the TVU findings and hormone levels.

The bleeding pattern was least favorable in the Velmari® group with a mean value of only 76 bleeding-free days, compared with 78 days for T3 (20 μg/2 mg), 87 days for T2 (10 μg/2 mg), and 89 days for T1 (EE/DNG 10 μg/1 mg), TC1 to TC4 inclusive.

Also, during the TC1-TC3 period, the PR formulations of the invention presented a more favorable profile with respect to the number of “heavy” bleeding, as well as for “spotting” and “no bleeding” days.

All products were safe and well tolerated. One single serious AE reported in this study was unrelated to the investigational medicinal product (IMP) and occurred under Velmari® treatment. The safety data did not reveal any clinically meaningful differences between the treatment groups.

The Phase Ill studies described herein demonstrate that the methods for oral contraception of the present invention are capable of providing surprisingly improved contraceptive efficacy based on overall Pearl Index (PI), as well as surprisingly improved cycle control in terms of bleeding profiles compared to known contraceptive methods.

Low doses of EE<0.02 mg per day in oral contraceptives are associated with more bleeding (Stanczyk et al. 2024). However, the methods for oral contraception of the present invention provides a clinically superior bleeding profile (less than −2.5 bleeding days per cycle at cycle 9) than currently marketed COCs containing immediate-release DNG 3 mg/EE 0.02 mg, which has a mean of 3.2-6.2 days bleeding per cycle in two trials of 12 cycles in >1500 participants (Valette SmPC, 2018).

The prolonged-release DNG/EE formulations of present invention aim to reduce the peak of exposure compared with immediate-release formulations (with maximal concentration [Cmax] occurring after 1.5 hours) and to delay the time to maximal concentration [Tmax] by a few hours to achieve a global similar total exposure with reduced peak intensity and reduced fluctuations in systemic blood levels of the active ingredients.

Without wishing to be bound to any theory, it is hypothesised that the improved cycle control with the prolonged-release DNG/EE formulations of present invention is due to reduced fluctuations in serum levels of the active ingredients. Because Tmax is delayed by using the prolonged-release formulations of the present disclosure, administration of the scheduled active daily oral dosage units can be missed, skipped or delayed for up to 24 hours from the scheduled daily administration without reducing protection against pregnancy. By contrast, the commercially available immediate-release contraceptive Valette, containing 2 mg DNG and 0.03 mg EE, permits a delay of only up to 12 hours from the scheduled daily administration.

Accordingly, the first aspect of the invention relates to methods for oral contraception comprising: (i) a first phase wherein active daily dosage units of an oral contraceptive composition are administered to the female subject over a period of 21 to 27 consecutive days; and (ii) a second phase wherein no contraceptive composition is administered to the female subject over a period of 1 to 7 consecutive days, wherein: the oral contraceptive composition comprises 2 mg of 17α-cyanomethyl-17-β-hydroxyestra-4,9-dien-3-one (dienogest, DNG) and 0.02 mg of 17α-ethinylestradiol (ethynyl estradiol, EE), wherein the DNG and the EE are both subject to controlled release; and wherein protection against pregnancy is not reduced when administration of the active daily oral dosage units is missed, skipped or delayed for up to 24 hours from the scheduled daily administration.

In a further aspect, the method for oral contraception of present invention provides an overall Pearl Index of contraception that is less than 0.7 with a confidence interval (CI) of at least 95%. In some embodiments the overall Pearl Index of contraception is less than 0.2 with a confidence interval (CI) of at least 95%.

In a further aspect, the present invention also provides methods for treating of one or more of acne, endometriosis, dysmenorrhea, dysfunctional uterine bleeding, cycle dependent complaints or uterine fibroids in a female subject.

### BRIEF DESCRIPTION OF THE DRAWINGS

FIG. 1A: Dienogest (DNG) dissolution profile of Formulation 1.

FIG. 1B: Ethinylestradiol (EE) dissolution profile of Formulation 1

FIG. 2A: Dienogest (DNG) dissolution profiles of Formulations 2 and 3.

FIG. 2B: Ethinylestradiol (EE) dissolution profiles of Formulations 2 and 3.

FIG. 3A: Dienogest (DNG) dissolution profiles of Formulations 4 and 5.

FIG. 3B: Ethinylestradiol (EE) dissolution profiles of Formulations 4 and 5.

FIG. 4A: Dienogest (DNG) dissolution profiles of Formulations 6 to 8.

FIG. 4B: Ethinylestradiol (EE) dissolution profiles of Formulations 6 to 8.

FIG. 5: Mean (arithmetic) dienogest plasma concentration-time profile (linear) after oral administration of a multiple dose once daily for 7 days.

FIG. 6: Mean (arithmetic) ethinylestradiol plasma concentration-time profile (linear) after oral administration of a multiple dose once daily for 7 days.

FIG. 7: Dienogest plasma concentration-time profile on dosing days 1 to 7 (before kinetic day 7). Legend: TEST 1: 2 mg dienogest and 20 μg ethinylestradiol per modified release tablet; TEST 2: 1 mg dienogest and 10 μg ethinylestradiol per modified release tablet; TEST 3: 2 mg dienogest and 10 μg ethinylestradiol per modified release tablet; REFERENCE: 2 mg dienogest and 30 μg ethinylestradiol per immediate release tablet.

FIG. 8: Ethinylestradiol plasma concentration-time profile on dosing days 1 to 7 (before kinetic day 7). Legend: TEST 1: 2 mg dienogest and 20 μg ethinylestradiol per modified release tablet; TEST 2: 1 mg dienogest and 10 μg ethinylestradiol per modified release tablet; TEST 3: 2 mg dienogest and 10 μg ethinylestradiol per modified release tablet; REFERENCE: 2 mg dienogest and 30 μg ethinylestradiol per immediate release tablet.

FIG. 9: Bleeding Pattern TC1 to TC3, Mean Number of Days (Full Analysis Set). Abbreviations: DNG=dienogest; EE=ethinyl estradiol; FAS=full analysis set; N=number of subjects; T1=EE/DNG 10 μg/1 mg; T2=EE/DNG 10 μg/2 mg; T3=EE/DNG 20 μg/2 mg; TC=treatment cycle; Velmari®=Velmari Langzyklus 0.02/3 mg tablets (EE 20 μg/drospirenone 3 mg).

FIG. 10: Bleeding Pattern TC1 to TC4, Mean Number of Days (Full Analysis Set). Abbreviations: DNG=dienogest; EE=ethinyl estradiol; FAS=full analysis set; N=number of subjects; T1=EE/DNG 10 μg/1 mg; T2=EE/DNG 10 μg/2 mg; T3=EE/DNG 20 μg/2 mg; TC=treatment cycle; Velmari®=Velmari Langzyklus 0.02/3 mg tablets (EE 20 μg/drospirenone 3 mg).

FIGS. 11A & 11B: Charts showing proportion of participants with unscheduled bleeding during cycles 2-9 depending on oral contraceptive composition.

FIGS. 12A & 12B: Charts showing mean number of days for subjects with unscheduled bleeding and/or spotting depending on the oral contraceptive.

FIG. 13: Charts shown proportion of subjects with Prolonged Beeding (>10 consec. days) in cycles 2-4, 4-7 and 8-9 depending on the oral contraceptive.

### DETAILED DESCRIPTION

### Definitions

As used herein, the term “dienogest (DNG)” or “17α-cyanomethyl-17-β-hydroxyestra-4,9-dien-3-one” is defined for purposes of the invention as comprising (i) unsalified dienogest (also known as dienogest base), its pharmaceutically acceptable salts and mixtures thereof; as well as (ii) esters, solvates, complexes, polymorphs, hydrates or prodrugs of (i) as used herein. Preferably, the dienogest in the composition is unsalified dienogest, a pharmaceutically acceptable salt thereof, or a mixture thereof. Dienogest has PubChem CID number: 68861.

As used herein, the term “ethynyl estradiol (EE)” or “17α-ethinylestradiol” is defined for purposes of the invention as comprising (i) unsalified ethynyl estradiol (also known as ethynyl estradiol base), its pharmaceutically acceptable salts and mixtures thereof; as well as (ii) esters, solvates, complexes, polymorphs, hydrates or prodrugs of (i) as used herein. Preferably, the ethinyl estradiol in the composition is unsalified ethynyl estradiol, a pharmaceutically acceptable salt thereof, or a mixture thereof. Ethynyl estradiol has PubChem CID number: 5991.

As used herein, the term “on-drug pregnancy” is defined for purposes of the invention as all conceptions that occur from day 1 (the initiation of contraceptive administration) through seven days after the final tablet (active or placebo) is taken.

As used herein, the term “exposure cycle” is defined for purposes of the invention as a 28-day treatment cycle. As used herein, the term “evaluable cycle” is defined for the purposes of the invention as an exposure cycle with intercourse without use of back-up contraceptive during the cycle.

Esters of DNG and/or EE may be prepared through functionalization of hydroxyl and/or carboxyl groups that may be present within the molecular structure of the compound (Advanced Organic Chemistry: Reactions, Mechanisms and Structure, 4th Ed. (New York: Wiley-Interscience, 1992).

Pharmaceutically acceptable salts include those formed with free amino groups such as those derived from hydrochloric, phosphoric, acetic, oxalic, tartaric acids and the like, and those formed with free carboxyl groups such as those derived from sodium, potassium, ammonium, calcium, ferric hydroxides, isopropylamine, triethylamine, 2-ethylamino ethanol, histidine, procaine or similar.

As used herein, the mass of DNG and/or EE in any dosage form of the invention or of a test, reference, control or comparator dosage form comprising DNG and/or EE refers to the amount (mass) of unsalified DNG and/or EE, or a pharmaceutically salt of DNG and/or EE, or a mixture thereof.

As used herein with respect to the dosage form of the invention, the term “oral”, “oral dosage form”, “oral pharmaceutical dosage form”, “oral administration”, “oral compositions” “oral pharmaceutical compositions”, “oral contraceptive compositions”, “oral tablets”, “oral capsules”, “orally ingested”, “orally”, “oral route” and the like all refer to any method of administration through the mouth. The oral dosage form of the invention is usually ingested intact, although it may be ingested tampered (e.g., crushed) and usually with the aid of water or a beverage to hasten passage through the mouth.

As used herein, the term “extended-release” dosage forms mean pharmaceutical preparations which release an active ingredient from a dosage form or a portion thereof in other than an immediate release fashion. Extended-release pharmaceutical compositions are made by incorporating a controlled release material (e.g., controlled release excipients) in the dosage form. Extended-release dosage forms are sometimes designed to accomplish pharmaceutical, pharmacokinetic, pharmacodynamic, therapeutic or convenience objectives not offered by conventional dosage forms such as a solution or an immediate release dosage form.

As used herein, the term “extended-release” is interchangeably with “modified release”, “controlled release”, “prolonged release”, “slow release”, “sustained release”, “long acting” and the like. Extended-release dosage forms release the active ingredient from a dosage form or a portion thereof over an extended period of time (over a period of time of 4, 6 hours or 8 hours or greater, preferably over for period greater than about 8 hours, and most preferably over for period greater than about 10 hours, 12, 14, 16, 18, 20, 22 or 24 hours. Extended-release dosage forms may be either delayed onset formulations, i.e., “delayed onset, extended-release” (e.g., a delay in release of 1, 1.5, 2, 2.5, 3, 3.5, 4, 4.5, 5, 5.5, 6, 6.5, 7, 7.5 or 8 hours after ingestion, preferably at least 2 hours after ingestion) or preferably “extended-release” (i.e., without a significant initial delay in release). In some embodiments, these extended-release compositions are formulated to be suitable for daily administration.

As used herein, “controlled release material”, “controlled release means”, “rate controlling means”, “rate controlling excipient”, “rate controlling ingredient”, “rate controlling material”, “release rate controlling means”, “release rate controlling excipient”, “release rate controlling ingredient”, “release rate controlling material”, and “material to provide controlled release” means an in vitro or in vivo release rate controlling excipient or material incorporated in the dosage form whose function or primary function is to modify release (e.g, onset of release, rate of release, duration of release) of an active drug (e.g., DNG and/or EE) from a dosage form or a portion (i.e., cause the dosage form to release in other than an immediate release fashion). In more preferred embodiments of the invention, the controlled release material functions to provide one or more of the following, compared to immediate release DNG and/or EE: (1) change in the onset of release; (2) change in the rate of release; (3) change in the duration of release; (4) change in the time of peak plasma concentration; (5) change in the peak plasma concentration; (6) change in the extent of absorption; (7) change in the onset of therapeutic effect; (8) change in the duration of therapeutic effect; and (9) change in the gastrointestinal anatomic location of release.

As used herein, the terms “missed”, “skipped” and delayed are interchangeable and refer to when one or more oral dosage units of a contraceptive composition are not administered to a subject at the time prescribed by a method of contraception, but the one or more missed, skipped or delayed oral dosage units are administered at a later time but within 24 hours after said skipped, missed or delayed dosage unit should have been administered.

As used herein a contraceptive method relates to a method for preventing pregnancy.

As used herein, “treatment”, “treating” or “treat” refer to: (i) preventing or retarding a disease, disorder or condition from occurring in a subject which may be predisposed to the disease, disorder and/or condition but has not yet been diagnosed as having it; (ii) inhibiting the disease, disorder or condition, i.e., arresting or slowing down its development or progression; and/or (iii) relieving the disease, disorder or condition, i.e., causing regression of the disease, disorder and/or condition. In certain embodiments, such term refers to the amelioration or eradication of a disease or symptoms associated with a disease.

As used herein a “therapeutically effective amount” refers to an amount effective, at dosages and for periods of time necessary to achieve the desired therapeutic result, such as one or more of the following therapeutic results, such as a significant delay of the onset or progression of the disease; or a significant reduction of the severity of one or more symptoms. A therapeutically effective amount is also typically one in which any toxic or detrimental effect of the active ingredient or pharmaceutical composition is outweighed by the therapeutically beneficial effects.

### Contraceptive Methods

One aspect of the present invention is the use of the oral contraceptive compositions described herein in contraception; in other words, it provides the oral composition as described herein as a contraceptive composition. When used for contraceptive purposes, said oral composition is used in a female subject of child-bearing age, i.e., from the puberty to the menopause. Women of child-bearing age also include women in peri-menopause.

A related aspect pertains to an oral contraceptive method for a female subject in need thereof characterized in that it comprises the step of administering active daily dosage units of an oral composition as described herein to said female subject over a period of several consecutive days. In some embodiments, the active daily dosage unit is able to inhibit ovulation when daily administered to a female patient over the selected period, in preferred embodiments wherein said daily dosage unit is administered for 21 to 28 consecutive days.

One embodiment relates to methods for oral contraception comprising: (i) a first phase wherein active daily dosage units of an oral contraceptive composition are administered to the female subject over a period of 21 to 27 consecutive days; and (ii) a second phase wherein no contraceptive composition is administered to the female subject over a period of 1 to 7 consecutive days, wherein: the oral contraceptive composition comprises 2 mg of 17α-cyanomethyl-17-β-hydroxyestra-4,9-dien-3-one (dienogest, DNG) and 0.02 mg of 17α-ethinylestradiol (ethynyl estradiol, EE), wherein at least one of the DNG and the EE are subject to controlled release; and protection against pregnancy is not reduced when administration of the active daily oral dosage units is missed, skipped or delayed for up to 24 hours from the scheduled daily administration.

The Pearl Index (PI), i.e. the number of pregnancies per 100 women per year of treatment, is a common calculation used in clinical trials for reporting the effectiveness of a birth control method. In general, a higher PI means a product is less effective at preventing pregnancy compared to a product with a lower PI. While method failures occur during ‘perfect use’, subject failures (non-compliance) occur during ‘typical use’. The overall PI includes pregnancies due to method and subject failures. As used herein the “overall PI” is calculated as the number of confirmed on-drug pregnancies divided by cumulative cycles of exposure with the quotient multiplied by 1300.

In an embodiment, the overall Pearl Index of the oral contraceptive composition is less than 4, preferably less than 2, wherein the overall Pearl Index is calculated as the number of confirmed on-drug pregnancies divided by cumulative cycles of exposure with the quotient multiplied by 1300. In an embodiment, the overall Pearl Index of the oral contraceptive composition is less than 0.7 with a confidence interval (CI) of at least 95%, wherein the overall Pearl Index is calculated as the number of confirmed on-drug pregnancies divided by cumulative cycles of exposure with the quotient multiplied by 1300. In some embodiments an overall Pearl Index of contraception is less than 0.2, with a confidence interval (CI) of at least 95%, wherein the overall Pearl Index is calculated as the number of confirmed on-drug pregnancies divided by cumulative cycles of exposure with the quotient multiplied by 1300.

In an embodiment, the oral contraceptive composition comprises 2 mg of 17α-cyanomethyl-17-β-hydroxyestra-4,9-dien-3-one (dienogest, DNG) and 0.02 mg of 17α-ethinylestradiol (ethynyl estradiol, EE), in an extended-release matrix.

In an embodiment, DNG and EE are the sole contraceptive ingredients in said daily dosage units. Preferably, the dosage units do not comprise any other active ingredient.

The number of days for the administration of said daily dosage units can be 21, 22, 23, 24, 25 or 26 and the number of days free or of placebo dosages intake then 7, 6, 5, 4, 3 or 2 respectively, in the 28-day menstrual cycle. In a particular embodiment, said daily dosage unit is administered for 24 consecutive days followed by a 4-day hormone free period. In some other embodiments, the contraceptive method of the invention consists in administering “continuously” daily dosage units of the invention. Such a method does not comprise a free-contraceptive period i.e. a period in which no contraceptive or placebo pill is administered. In further embodiments the number days of administration of the daily dosage units is of 28 or a multiple of 28, for example 2 to 3 times 28.

In additional embodiments, said method comprises administering the daily dosage units for an extended regimen with hormone free interval every 6 months or one year. In still further embodiments, said method comprises administering the daily dosage units from 87 to 120 consecutive days, followed by one 3 or 4-day hormone free period.

As used herein a period of 1 to 7 consecutive days include periods of 1 day, of 2 consecutive days, of 3 consecutive days, of 4 consecutive days, of 5 consecutive days, of 6 consecutive days, and of 7 consecutive days.

As used herein a period of 21 to 27 consecutive days include periods of 21 consecutive days, of 22 consecutive days, of 23 consecutive days, of 24 consecutive days, of 25 consecutive days, of 26 consecutive days, and of 27 consecutive days. As mentioned above, the duration of the first phase plus the second phase is preferably 28 days.

In some embodiments the first phase lasts from 21 to 24 consecutive days, and the second phase lasts from 4 to 7 consecutive days. In some embodiments the first phase lasts 24 consecutive days, and the second phase lasts from 4 consecutive days.

In the first phase, the composition of active daily dose units may remain constant or may vary, with respect to the daily amount of EE and/or DNG. Preferably, it remains constant.

The second phase is a free-contraceptive period i.e. a phase during which no contraceptive ingredients is administered to the female subject. During the said second phase, daily placebo dosage units may be administered to the female subject. In some other cases, no pill is administered to the female subject. Said second phase may enable regular menstrual bleedings to occur and thus may enable to mimic the natural menstrual cycle.

As used herein, the term “active daily dosage unit” refers to physically discrete units suitable as unitary dosage which consists of a contraceptive composition as fully described hereabove in the present specification.

As used herein the term “scheduled daily administration” means that the active daily dosage units of the oral contraceptive composition are administered to the female subject every day at about the same time, i.e. every 24 hours.

Another aspect relates to improved contraception even when the administration of the active daily doses of the oral contraceptive composition is missed, skipped or delayed for up to 24 h from the scheduled daily administration.

For example, in some embodiments administration of a scheduled daily oral dose can be missed, skipped or delayed for up to 24 hours from the scheduled daily administration without reducing protection against pregnancy. In some embodiments two daily oral doses are then administered within 24 hours from the scheduled daily administration of the one scheduled daily oral dose that is missed, skipped or delayed. In other embodiments administration of two non-consecutive scheduled daily oral doses can be missed, skipped or delayed for up to 24 hours from the scheduled daily administration. Two daily oral doses may then be administered within 24 hours from each scheduled daily administration of the two non-consecutive scheduled daily oral doses that are missed, delayed or skipped.

Another aspect relates to improved contraception even when the administration of an active daily dose unit of the oral contraceptive composition is missed, skipped or delayed for more than 24 h from the scheduled daily administration between days 8 and 14 of administration. In such cases, protection against pregnancy is not reduced provided that in the 7 days preceding the missed, skipped or delayed active daily dose unit of the oral contraceptive composition, the female subject has taken all active daily dosage units according to the scheduled daily administration. In some embodiments two daily oral doses are then administered together and the remaining active daily dose unit of the oral contraceptive composition are administered according to the scheduled daily administration.

As explained above, methods of the present disclosure can yield surprisingly high levels of contraceptive efficacy. One measure of this increased contraceptive efficacy relates to significant reductions in the overall Pearl Index (PI) of contraception.

In some embodiments the overall Pearl Index of contraception is less than 0.7 with a confidence interval (CI) of at least 95%, when administration of up to two non-consecutive active daily oral dosage units are skipped or delayed for up to 24 hours from the scheduled daily administration. In some embodiments the method provides a reduced overall Pearl Index (PI) of contraception compared to orally administering a progestogen-only contraceptive composition. In some embodiments the method provides a reduced overall Pearl Index (PI) of contraception compared to orally administering a contraceptive composition comprising 3 mg of 17β-Hydroxy-6β,7β:15β,16β-dimethylene-3-oxo-17α-pregn-4-ene-21-carboxylic acid, γ-lactone (drospirenone, DRSP) and 0.02 mg of 17α-ethinylestradiol (ethynyl estradiol, EE). In some embodiments the method provides a reduced overall Pearl Index (PI) of contraception compared to orally administering a contraceptive composition comprising 2 mg of 17α-cyanomethyl-17-β-hydroxyestra-4,9-dien-3-one (dienogest, DNG) and 0.03 mg of 17α-ethinylestradiol (ethynyl estradiol, EE).

In some embodiments the overall Pearl Index of contraception is less than 0.7, with a confidence interval (CI) of at least 95%, when the method is carried out over a period of 7 to 9 menstruation cycles. In some embodiments the overall Pearl Index of contraception is less than 0.6, with a confidence interval (CI) of 95%. In some embodiments the overall Pearl Index of contraception is less than 0.4, with a confidence interval (CI) of 95%. In some embodiments the overall Pearl Index of contraception is less than or equal to 0.2, with a confidence interval (CI) of 95%.

As explained above, methods of the present disclosure can also yield surprisingly improved tolerability in terms of improved bleeding profile compared to known contraceptive methods.

Methods of the present disclosure can provide an improved bleeding profile in the female subject compared to orally administering a progestogen-only contraceptive composition. In some embodiments the method provides an improved bleeding profile in the female subject compared to orally administering a contraceptive composition comprising 3 mg of 17β-Hydroxy-6β,7β:15β,16β-dimethylene-3-oxo-17α-pregn-4-ene-21-carboxylic acid, γ-lactone (drospiren-one, DRSP) and 0.02 mg of 17α-ethinylestradiol (ethynyl estradiol, EE). In some embodiments the method provides an improved bleeding profile in the female subject compared to orally administering a contraceptive composition comprising 2 mg of 17α-cyanomethyl-17-β-hydroxyestra-4,9-dien-3-one (dienogest, DNG) and 0.03 mg of 17α-ethinylestradiol (ethynyl estradiol, EE).

In some embodiments the improved bleeding profile provided by the inventive method comprises reduced incidences of spotting and/or bleeding. For example, in some embodiments when the oral contraceptive composition is administered to a plurality of female subjects over a period of at least 9 menstruation cycles, no more than 90% of the female subjects experience bleeding, spotting, or a combination thereof, during menstruation cycles 2-9.

In some embodiments, the improved bleeding profile provided by the inventive methods results in a reduced number of bleeding/spotting days per cycle. For example, in some embodiments when the oral contraceptive composition is administered to a plurality of female subjects over a period of at least 9 menstruation cycles, the female subjects experience less than 5 days of bleeding/spotting per cycle.

In some embodiments the improved bleeding profile provided by the inventive method comprises reduced incidences of unscheduled spotting and/or bleeding. For example, in some embodiments when the oral contraceptive composition is administered to a plurality of female subjects over a period of at least 9 menstruation cycles, no more than 60% of the female subjects experience unscheduled bleeding, unscheduled spotting, or a combination thereof, during menstruation cycles 2-9.

In some embodiments when the oral contraceptive composition is administered to a plurality of female subjects over a period of at least 6 menstruation cycles, no more than 55% of the female subjects experience unscheduled bleeding, unscheduled spotting, or a combination thereof, during menstruation cycles 2-6.

In some embodiments when the oral contraceptive composition is administered to a plurality of female subjects over a period of at least 9 menstruation cycles, no more than 90% of the female subjects experience scheduled bleeding, scheduled spotting, or a combination thereof, during menstruation cycles 2-9.

In some embodiments when the oral contraceptive composition is administered to a plurality of female subjects over a period of at least 9 menstruation cycles, no more than 50% of the female subjects experience frequent bleeding, frequent spotting, or a combination thereof, during menstruation cycles 2-9.

In some embodiments when the oral contraceptive composition is administered to a plurality of female subjects over a period of at least 9 menstruation cycles, no more than 55% of the female subjects experience irregular bleeding, irregular spotting, or a combination thereof, during menstruation cycles 2-9.

In some embodiments when the oral contraceptive composition is administered to a plurality of female subjects over a period of at least 9 menstruation cycles, no more than 55% of the female subjects experience prolonged bleeding lasting more than ten consecutive days, prolonged spotting lasting more than ten consecutive days, or a combination thereof, during menstruation cycles 2-9.

In some embodiments when the oral contraceptive composition is administered to a plurality of female subjects over a period of at least 9 menstruation cycles, at least 10% of the female subjects experience absence of bleeding, absence of spotting, or a combination thereof, during menstruation cycles 2-9.

### Oral Compositions

Another aspect of the invention pertains to an oral composition (preferably, an oral contraceptive composition) comprising 2 mg of DNG and equal to or less than 0.02 mg of EE, wherein the pharmaceutical form of said composition is a modified release form wherein at least 80%, preferably at least 85%, at least 90%, at least 95%, at least 97%, at least 99%, and more preferably all the content of EE is intended for slow or controlled release.

In preferred embodiments, at least 80%, preferably at least 85%, at least 90%, at least 95%, at least 97% or at least 99% %, and more preferably all the content of DNG and EE, wherein each is independently selected, is intended for slow or controlled release; preferably all the content of DNG and EE is intended for slow or controlled release.

In a preferred embodiment, the modified release oral contraceptive composition of the invention comprises an extended-release matrix.

In some embodiments, the dosage form of the invention is an oral dosage form (preferably a tablet) comprising: (i) 2 mg of DNG and equal to or less than 0.02 mg of EE, and (ii) controlled release material to render said dosage form suitable for extended-release, preferably said dosage form suitable for dosing every 24 hours. In other words, in preferred embodiments, the composition as described herein is a daily dosage form. Furthermore, the slow or controlled release dosage forms of the present invention may preferably release EE, preferably EE and DNG, at a rate that is independent of pH, e.g., between pH 1.6 and 7.2. In other words, the dosage forms of the present invention avoid “dose dumping” upon oral administration.

Oral contraception compositions of the invention may contain 2 mg of DNG and equal to or less than 0.02 mg of EE, preferably from 0.01 mg to 0.02 mg of EE, including 0.0125, 0.015, or 0.0175, more preferably 0.02 mg of EE. Said composition may contain other active ingredients. Preferably, does not contain other active ingredients with contraceptive effects. In preferred embodiments, DNG and EE are the only active ingredients in the composition.

In some embodiments, the composition of the invention is further characterized by its pharmacokinetic profile. For a given DNG and EE-containing composition, the DNG and/or EE plasma concentration versus time curve may be determined by following plasma DNG and/or EE concentration over a period of about 72 h after a single oral intake of one daily dosage unit of the said composition. Alternatively, said pharmacokinetic profile may also be obtained over a period of 7 days after a daily oral intake of said composition. The AUC₀ₕ₋ₜₗₐₛₜ, the AUC(0-τ), the Cₘₐₓ and the tₘₐₓ are determined based on the DNG or EE plasma concentration versus time curve.

The oral administration of said DNG- and EE-containing composition is preferably performed in fasting conditions i.e. without food and not close to mealtime (i.e. in general, approximately 6 h-10 h after meal) since food ingestion may modify the absorption rate of drospirenone in the gastrointestinal tract. Generally, the study population is composed by healthy premenopausal females, including women in peri-menopause.

In some preferred embodiments, the pharmacokinetic and pharmacodynamic parameters of the specifications and claims are determined under fed conditions. In other preferred embodiments, the pharmacokinetic and pharmacodynamic parameters of the specifications and claims are determined under fast conditions. Preferably, these are determined under fast conditions.

In preferred embodiments, the tₘₐₓ and Cₘₐₓ values refer to the maximum DNG or EE plasma concentration and the time to reach it, respectively, after the oral administration of a daily dosage unit of the DNG and EE-containing composition during 7 days. This tₘₐₓ and Cₘₐₓ may reflect the mean tₘₐₓ and Cₘₐₓ, respectively, of a population under study and may be the arithmetic or geometric mean, preferably the arithmetic mean. In particularly preferred embodiments, said composition is adapted to provide a pharmacokinetic profile for EE characterized by a Tmax from 3.5 h to 4 h, preferably around 3.75 h, further to oral administration to a human premenopausal female under fasting conditions once daily for 7 days.

In some embodiments, optionally in combination with any of the above, the oral composition presents a pharmacokinetic profile further to oral administration under fasting conditions once daily for 7 days characterized by: Tmax of DNG from 3.5 h to 4 h, preferably around 3.75 h; and a Tmax of EE from 3.5 h to 4 h, preferably around 3.75 h.

Preferably, the pharmacokinetic profile of said composition further to oral administration under fasting conditions once daily for 7 days is further characterized by a Cₘₐₓ of EE from 60 μg/mL to 65 μg/mL. In preferred embodiments, it is further characterized by a Cₘₐₓ of EE from 60 μg/mL to 65 μg/mL and a Cₘₐₓ of DNG from 55 ng/mL to 60 ng/mL.

The AUC(0-τ) (=AUCtₗₐₛₜ) refers to the area under the concentration/time curve, calculated by the trapezoidal rule from time 0 h to last observed concentration at time t. The term AUC(0-τ) as used herein refers to the area under the concentration-time curve during a dosing interval. The AUC(0-t) may reflect the mean AUC(0-τ), of a population under study and may be the arithmetic or geometric mean, preferably the arithmetic mean. In some embodiments, optionally in combination with any of the above, said composition is further characterized by having an AUC(0-τ) for EE from 680 to 710 ng*h/mL further to oral administration under fasting conditions once daily for 7 days, preferably said AUC(0-τ) is from 680 to 710 ng*h/mL and the AUC(0-τ) of DNG is from 710 to 740 ng*h/mL.

In some embodiments, after single-dose oral administration to the female subject, the oral contraceptive composition provides: a Cₘₐₓ of EE of less than 75 μg/mL; and a Cₘₐₓ of DNG of less than 70 ng/mL.

In some embodiments, after single-dose oral administration to the female subject, the oral contraceptive composition provides: a Cₘₐₓ of EE ranging from 29.2 μg/mL to 36.9 μg/mL; and a Cₘₐₓ of DNG ranging from 34.9 ng/mL to 48.5 ng/mL.

In some embodiments, after single-dose oral administration to the female subject under fasting conditions, the oral contraceptive composition provides: a Tmax of EE ranging from 2.0 hours to 5.0 hours; and a Tmax of DNG ranging from 1.5 hours to 6.0 hours.

In some embodiments, after single-dose oral administration to the female subject, the oral contraceptive composition provides: a Tmax of EE ranging from 3.1 hours to 4.7 hours; and a Tmax of DNG ranging from 3.0 hours to 4.4 hours.

In some embodiments, after single-dose oral administration to the female subject, the oral contraceptive composition provides: an AUC_{0-72 hr} of EE of at least 200 pg×h/mL; and an AUC_{0-72 hr} of DNG of at least 300 ng×h/mL.

In some embodiments, after single-dose oral administration to the female subject, the oral contraceptive composition provides: an AUC_{0-72 hr} of EE ranging from 435 pg×h/mL to 562 pg×h/mL; and an AUC_{0-72 hr} of DNG ranging from 545 ng×h/mL to 689 ng×h/mL.

In some embodiments, after single-dose oral administration to the female subject, the oral contraceptive composition provides: a Cₘₐₓ of EE of less than 75 μg/mL; and a Cₘₐₓ of DNG of less than 70 ng/mL; a Tmax of EE ranging from 2.0 hours to 5.0 hours; and a Tmax of DNG ranging from 1.5 hours to 6.0 hours; and an AUC_{0-72 hr} of EE of at least 200 pg×h/mL; and an AUC_{0-72 hr} of DNG of at least 300 ng×h/mL.

In some embodiments, after single-dose oral administration to the female subject, the oral contraceptive composition provides: a Cₘₐₓ of EE ranging from 29.2 μg/mL to 36.9 μg/mL; a Tmax of EE ranging from 3.1 hours to 4.7 hours; an AUC_{0-72 hr} of EE ranging from 435 pg×h/mL to 562 pg×h/mL; a Cₘₐₓ of DNG ranging from 34.9 ng/mL to 48.5 ng/mL; a Tmax of DNG ranging from 3.0 hours to 4.4 hours; and an AUC_{0-72 hr} of DNG ranging from 545 ng×h/mL to 689 ng×h/mL.

In some embodiments, after once daily oral administration to the female subject under fasting conditions for 7 days, the oral contraceptive composition provides: a Cₘₐₓ of EE ranging from 60 μg/mL to 65 μg/mL; and a Cₘₐₓ of DNG ranging from 55 ng/mL to 60 ng/mL.

In some embodiments, after once daily oral administration to the female subject under fasting conditions for 7 days, the oral contraceptive composition provides: a Tmax of EE ranging from 3.5 hours to 4.0 hours; and a Tmax of DNG ranging from 3.5 hours to 4.0 hours.

In some embodiments, after once daily oral administration to the female subject under fasting conditions for 7 days, the oral contraceptive composition provides: an AUC_{0-24 hr} of EE ranging from 680 pg×h/mL to 710 pg×h/mL; and an AUC_{0-24 hr} of DNG ranging from 710 ng×h/mL to 740 ng×h/mL.

In some embodiments, after once daily oral administration to the female subject under fasting conditions for 7 days, the oral contraceptive composition provides: a Cₘₐₓ of EE ranging from 60 μg/mL to 65 μg/mL; a Tmax of EE ranging from 3.5 hours to 4.0 hours; an AUC_{0-24 hr} of EE ranging from 680 pg×h/mL to 710 pg×h/mL; a Cₘₐₓ of DNG ranging from 55 ng/mL to 60 ng/mL; a Tmax of DNG ranging from 3.5 hours to 4.0 hours; and an AUC_{0-24 hr} of DNG ranging from 710 ng×h/mL to 740 ng×h/mL.

In some embodiments, after single-dose oral administration to the female subject under fasting conditions, the oral contraceptive composition provides: a Cₘₐₓ of EE of less than 65 μg/mL; and a Cₘₐₓ of DNG of less than 65 ng/mL.

In some embodiments, after single-dose oral administration to the female subject under fasting conditions, the oral contraceptive composition provides: a Cₘₐₓ of EE ranging from 29.2 μg/mL to 35.6 μg/mL; and a Cₘₐₓ of DNG ranging from 34.9 ng/mL to 42.7 ng/mL.

In some embodiments, after single-dose oral administration to the female subject under fasting conditions, the oral contraceptive composition provides: a Tmax of EE ranging from 3.0 hours to 5.0 hours; and a Tmax of DNG ranging from 3.0 hours to 6.0 hours.

In some embodiments, after single-dose oral administration to the female subject under fasting conditions, the oral contraceptive composition provides: a Tmax of EE ranging from 3.9 hours to 4.7 hours; and a Tmax of DNG ranging from 3.6 hours to 4.4 hours.

In some embodiments, after single-dose oral administration to the female subject under fasting conditions, the oral contraceptive composition provides: an AUC_{0-72 hr} of EE of at least 200 pg×h/mL; and an AUC_{0-72 hr} of DNG of at least 300 ng×h/mL.

In some embodiments, after single-dose oral administration to the female subject under fasting conditions, the oral contraceptive composition provides: an AUC_{0-72 hr} of EE ranging from 435 pg×h/mL to 531 pg×h/mL; and an AUC_{0-72 hr} of DNG ranging from 545 ng×h/mL to 665 ng×h/mL.

In some embodiments, after single-dose oral administration to the female subject under fasting conditions, the oral contraceptive composition provides: a Cₘₐₓ of EE of less than 65 μg/mL; and a Cₘₐₓ of DNG of less than 65 ng/mL; a Tmax of EE ranging from 2.0 hours to 5.0 hours; and a Tmax of DNG ranging from 1.5 hours to 6.0 hours and an AUC_{0-72 hr} of EE of at least 200 pg×h/mL; and an AUC_{0-72 hr} of DNG of at least 300 ng×h/mL.

In some embodiments, after single-dose oral administration to the female subject under fasting conditions, the oral contraceptive composition provides: a Cₘₐₓ of EE ranging from 29.2 μg/mL to 35.6 μg/mL; a Tmax of EE ranging from 3.9 hours to 4.7 hours; an AUC_{0-72 hr} of EE ranging from 435 pg×h/mL to 531 pg×h/mL; a Cₘₐₓ of DNG ranging from 34.9 ng/mL to 42.7 ng/mL; a Tmax of DNG ranging from 3.6 hours to 4.4 hours; and an AUC_{0-72 hr} of DNG ranging from 545 ng×h/mL to 665 ng×h/mL.

In some embodiments, after single-dose oral administration to the female subject under fed conditions, the oral contraceptive composition provides: a Cₘₐₓ of EE of less than 60 μg/mL; and a Cₘₐₓ of DNG of less than 65 ng/mL.

In some embodiments, after single-dose oral administration to the female subject under fed conditions, the oral contraceptive composition provides: a Cₘₐₓ of EE ranging from 30.2 μg/mL to 36.9 μg/mL; and a Cₘₐₓ of DNG ranging from 40.0 ng/mL to 48.5 ng/mL.

In some embodiments, after single-dose oral administration to the female subject under fed conditions, the oral contraceptive composition provides: a Tmax of EE ranging from 2.0 hours to 5.0 hours; and a Tmax of DNG ranging from 1.5 hours to 5 hours.

In some embodiments, after single-dose oral administration to the female subject under fed conditions, the oral contraceptive composition provides: a Tmax of EE ranging from 3.4 hours to 4.1 hours; and a Tmax of DNG ranging from 3.0 hours to 3.6 hours.

In some embodiments, after single-dose oral administration to the female subject under fed conditions, the oral contraceptive composition provides: an AUC_{0-72 hr} of EE of at least 200 pg×h/mL; and an AUC_{0-72 hr} of DNG of at least 300 ng×h/mL.

In some embodiments, after single-dose oral administration to the female subject under fed conditions, the oral contraceptive composition provides: an AUC_{0-72 hr} of EE ranging from 459 pg×h/mL to 562 pg×h/mL; and an AUC_{0-72 hr} of DNG ranging from 563 ng×h/mL to 689 ng×h/mL.

In some embodiments, after single-dose oral administration to the female subject under fed conditions, the oral contraceptive composition provides: a Cₘₐₓ of EE of less than 60 μg/mL; and a Cₘₐₓ of DNG of less than 65 ng/mL; a Tmax of EE ranging from 2.0 hours to 5.0 hours; and a Tmax of DNG ranging from 1.5 hours to 5 hours; and an AUC_{0-72 hr} of EE of at least 200 pg×h/mL; and an AUC_{0-72 hr} of DNG of at least 300 ng×h/mL.

In some embodiments, after single-dose oral administration to the female subject under fed conditions, the oral contraceptive composition provides: a Cₘₐₓ of EE ranging from 30.2 μg/mL to 36.9 μg/mL; a Tmax of EE ranging from 3.4 hours to 4.1 hours; an AUC_{0-72 hr} of EE ranging from 459 pg×h/mL to 562 pg×h/mL; a Cₘₐₓ of DNG ranging from 40.0 ng/mL to 48.5 ng/mL; a Tmax of DNG ranging from 3.0 hours to 3.6 hours; and an AUC_{0-72 hr} of DNG ranging from 563 ng×h/mL to 689 ng×h/mL.

In some embodiments, optionally in combination with any of the above, the composition of the invention is further characterized by its dissolution profile. In particular embodiments, the composition as described herein is characterized by EE, preferably DNG and EE, respectively, having a dissolution profile characterized in that: no more than 25% of the amount initially present in said composition is dissolved within 1 hour; and between 30% and 60% of the amount initially present in said composition is dissolved within 2 hours.

In preferred embodiments, at least 70%, preferably at least 80% of the amount initially present in said composition is dissolved within 8 hours. Preferably, within a range of 5 to 8 hours encompasses a time range of 5.5 to 8 hours, of 6 to 8 hours, of 6.5 to 8 hours, of 7 to 8 hours and of 7.5 to 8 hours. In particularly preferred embodiments, the composition is further characterized in that at least 70%, preferably at least 80% of the drospirenone is dissolved within 5 hours.

At least 70% of the EE and/or DNG encompasses at least 70%, at least 75%, at least 80%, at least 85%, at least 88%, at least 90%, at least 91%, at least 92%, at least 93%, at least 94%, at least 95%, at least 96%, at least 97%, at least 98%, at least 99%, at least 99.5% and 100%.

In particularly preferred embodiments, the composition as described herein is characterized by EE, preferably DNG and EE, respectively, having a dissolution profile characterized in that: no more than 25% of the amount initially present in said composition is dissolved within 1 hour; between 30% and 60% of the amount initially present in said composition is dissolved within 2 hours, and at least 70%, preferably at least 80% of the amount initially present in said composition is dissolved within 5 hours.

In some embodiments the in vitro dissolution rate of EE and/or DNG in the composition is assessed by the USP1 (baskets) method. Briefly, a tablet consisting of the composition comprising EE and DNG to be tested is placed in either 500 mL or 900 mL of water at 37° C. (±0.5° C.). The dissolution test is performed using a USP dissolution test apparatus 1 (baskets) at a stirring rate of 75 rpm. As used herein the phrase “Dissolution Method I” refers to the dissolution test using 900 mL of water; whereas the phrase “Dissolution Method II” refers to the dissolution test using 500 mL of water.

In some embodiments the DNG and the EE are formulated such that, when the oral contraceptive composition is subjected to an in vitro dissolution test according to USP1 (baskets) method using 500 mL of water at 37° C. (±0.5° C.) at a stirring rate of 75 rpm (Dissolution Method II), the DNG and the EE, respectively, exhibit a dissolution profile such that between 30% and 60% of the DNG initially present in the composition, and between 30% and 60% of the EE initially present in the composition, is dissolved within 2 hours.

In some embodiments the DNG and the EE are formulated such that, when the oral contraceptive composition is subjected to an in vitro dissolution test according to USP1 (basket) method using 500 mL of water at 37° C. (±0.5° C.) at a stirring rate of 75 rpm (Dissolution Method II), the DNG and the EE, respectively, exhibit a dissolution profile such that: (i) no more than 20% of the DNG initially present in the composition is dissolved within 0.5 hour; (ii) no more than 25% of the EE initially present in the composition is dissolved within 0.5 hour; (iii) between 30% and 60% of the DNG initially present in the composition is dissolved within 2 hours; and (iv) between 30% and 60% of the EE initially present in the composition is dissolved within 2 hours.

In some embodiments the DNG and the EE are formulated such that, when the oral contraceptive composition is subjected to an in vitro dissolution test according to USP1 (basket) method using 500 mL of water at 37° C. (±0.5° C.) at a stirring rate of 75 rpm (Dissolution Method II), the DNG and the EE, respectively, exhibit a dissolution profile such that: (i) no more than 20% of the DNG initially present in the composition is dissolved within 0.5 hour; (ii) no more than 25% of the EE initially present in the composition is dissolved within 0.5 hour; (iii) between 30% and 60% of the DNG initially present in the composition is dissolved within 2 hours; (iv) between 30% and 60% of the EE initially present in the composition is dissolved within 2 hours; (v) at least 70% of the DNG initially present in the composition is dissolved within 8 hours; and (vi) at least 70% of the EE initially present in the composition is dissolved within 8 hours.

In some embodiments the DNG and the EE are formulated such that, when the oral contraceptive composition is subjected to an in vitro dissolution test according to USP1 (basket) method using 500 mL of water at 37° C. (±0.5° C.) at a stirring rate of 75 rpm (Dissolution Method II), the DNG and the EE, respectively, exhibit a dissolution profile such that: (i) no more than 20% of the DNG initially present in the composition is dissolved within 0.5 hour; (ii) between 5% and 25% of the EE initially present in the composition is dissolved within 0.5 hour; (iii) between 37% and 57% of the DNG initially present in the composition is dissolved within 2 hours; (iv) between 37% and 57% of the EE initially present in the composition is dissolved within 2 hours; (v) at least 80% of the DNG initially present in the composition is dissolved within 8 hours; and (vi) at least 80% of the EE initially present in the composition is dissolved within 8 hours.

A composition with such an in vitro dissolution profile and/or the in vivo pharmacokinetic profile described above may be achieved in different ways.

The release of the slow-release or controlled-release proportion of EE, preferably of EE and DNG, can be controlled by a large number of controlled release means. Conventional forms of retardation include coating retardation and matrix retardation. In coating retardation, the core of a pharmaceutical composition containing an active ingredient is provided with a coating which consist of one or more hydrophylic and/or hydrophobic polymers and slows down release of the active ingredient. In matrix retardation, the active ingredient is contained in a matrix which is formed from one or more excipients and controls release of the active ingredient.

Examples of these release rate controlling excipients are inert plastics matrices, hydrocolloids, ion exchangers, slow-release coatings, gastro-resistant coatings, pellet mixtures, mixtures of minitablets and/or granules, microcapsules, osmotically controlled systems, erosion-controlled systems, diffusion-controlled systems and combinations thereof, fat- and wax-containing matrices.

In some embodiments, the oral dosage form comprises a plurality of pharmaceutically acceptable beads or pellets coated with the drug and overcoated with controlled release material. These beads or pellets may be compressed into tablets or filled into hard gelatin capsules. In other embodiments, the dosage form includes a capsule within a capsule, each capsule containing a different drug or the same drug(s). In some particular embodiments, the outer capsule may be an enteric coated capsule or a capsule containing an immediate release formulation to provide rapid plasma concentrations or a rapid onset of effect or a loading dose and the inner capsule contains an extended-release formulation. In one embodiment of the invention, the dosage form involves one or more tablets within a capsule, wherein the EE and/or DNG can be either in the tablet and/or in one of the capsules. In one embodiment of the invention, the composition is ingested orally as a tablet or capsule, preferably as a tablet.

In some other embodiments, the slow or controlled release behavior is achieved by an osmotically driven release system, wherein the oral dosage form may comprise (i) a drug layer; and (ii) a displacement layer comprising an osmopolymer; and (b) a semipermeable wall surrounding the bilayer core having a passageway disposed therein for the release of the drug(s). In some preferred embodiments, the oral dosage form comprises a compressed tablet, compressed capsule or uncompressed capsule. In some preferred embodiments, the oral dosage form comprises a tablet.

In some embodiments the oral contraceptive composition comprises EE and DNG, wherein the EE is dispersed within a matrix, preferably wherein EE and DNG are dispersed within a matrix. In certain preferred embodiments the oral dosage form of the present invention comprises a matrix which includes a controlled release material and EE, preferably EE and DNG. In certain preferred embodiments, the matrix is compressed into a tablet and may be optionally overcoated with a coating that in addition to the controlled release material of the matrix may control the release of the EE, preferably EE and DNG, from the formulation, such that blood levels of the active ingredient(s), are maintained within the therapeutic range over an extended period of time. In some embodiments, the coating is for immediate disintegration and release of the active ingredient. In certain alternative embodiments, the matrix is encapsulated. In some preferred embodiments, the extended-release oral dosage form of the present invention comprises a plurality of pharmaceutically acceptable extended-release matrices comprising EE, DNG or EE and DNG, the dosage form maintaining the blood plasma levels of the active ingredient(s) within the therapeutic range over an extended period of time when administered to patients.

In some embodiments, the dosage form of the invention comprises oral DNG and EE formulated to release the EE, preferably the EE and DNG, from the dosage form or to initiate the release of the EE, preferably the EE and DNG, from the dosage form after a certain specific amount of time post-oral ingestion, or at an approximately specific anatomic location in the gastrointestinal tract, or when the dosage form is in contact with specific gastrointestinal conditions (e.g., pH range, osmolarity, electrolyte content, food content, pressure, time since first ingestion, osmotic pressure in the dosage form, osmotic pressure in the gastrointestinal tract, hydration, etc), said dosage form suitable for providing an orally effective therapeutic for a short, intermediate or extended duration of effect, said dosage form providing a rapid or delayed onset of clinical effect. Preferably, said dosage form initiates the release of the EE, preferably the EE and DNG, immediately after ingestion.

In some embodiments of the invention, EE, preferably EE and DNG, is in the form of multiparticulates. In some embodiments of the invention, EE, preferably EE and DNG, is dispersed in a matrix. In some embodiments of the invention, EE, preferably EE and DNG, is in the form of multiparticulates that can be dispersed in a matrix or contained in a capsule. In some embodiments of the invention, EE, preferably EE and DNG, is in a matrix that is in the form of pellets. In some embodiments of the invention, EE, preferably EE and DNG is in coated beads. In some other embodiments of the invention, EE, preferably EE and DNG, is in the form of multiparticulates that are dispersed in a matrix and compressed into a tablet.

In some embodiments either DNG or EE may be present in the pharmaceutical composition of the invention in either micronized form or in non-micronized form. As used herein, the term “micronized” means a material that has been subjected to micronization. As used herein, the term “micronization” refers to a process of reducing the average particle size of a solid material, typically to provide a mixture of particles wherein more than 90% of particles have particle size of less than 15 μm, more preferably of less than 10 microns.

In some embodiments, the slow or controlled release behaviour of either DNG or EE is achieved by using non-micronized DNG and/or non-micronized EE with an appropriate particle size distribution. In such cases, the composition may optionally comprise other controlled release agents as described above. As used herein the term “non-micronized” means a material comprising a mixture of particles wherein more than 90% of particles have particle size (D₁₀) of more than 15 μm, preferably of more than 10 microns; and more than 90% of particles have particle size (D₉₀) of less than 250 μm, preferably of less than 200 μm.

In some particularly preferred embodiments, the dosage form of the invention comprises an oral formulation (e.g., tablet or capsule) which is coated to prevent substantial direct contact of EE, preferably EE and DNG, with the oral cavity (e.g. tongue, oral mucosa), oropharyngeal mucosal surface, esophagus or stomach. In some preferred embodiments, the dosage form of the invention comprises an oral formulation which is coated with a film or polymer.

In some embodiments, optionally in combination with any of the above, said extended-release form is a tablet and may be coated or not. Preferably, said tablet is a film-coated tablet comprising a tablet core and a film coating, wherein the tablet core comprises the content of EE for slow or controlled release, preferably wherein the tablet core comprises the content of DNG and EE for slow or controlled release. Preferably, the coating film allows for immediate disintegration for fast, active release. For instance, this film can be the one-step film coating system (Opadry™ II) which combines polymer, plasticizer and pigment which allows for immediate disintegration for fast, active release (Colorcon®|Opadry® II Complete Aqueous Film Coating System). In some embodiments, the extended-release EE, preferably EE and DNG, dosage form of the invention is a solid dispersion. By reducing drug particle size and therefore improving drug wettability, the bioavailability may be substantially improved. In addition, surfactants may be included to stabilize the dosage form in order to increase solubility and reduce recrystallization (see Vasconcelos et al, Drug Discovery Today, 2007; 12:1068-75, which is herein incorporated in its entirety by reference).

In some embodiments, EE (preferably EE and DNG) may be dispersed within an extended-release matrix as described herein above. The term “extended-release matrix” refers to one or more hydrophilic polymers and/or one or more hydrophobic polymers and/or one or more other type hydrophobic materials. In some embodiments, said extended-release matrix comprises one or more hydrophilic polymers and one or more hydrophobic polymers, such as in hydrophobic/hydrophilic matrix systems (PVAc/PVP). Suitable materials for inclusion in an extended-release matrix (also referred herein as polymeric matrix forming agents) are:
(a) Hydrophilic polymers include but are not limited to gums, cellulose ethers, hydrophilic acrylic polymers, ammonium alginate, sodium alginate, potassium alginate, calcium alginate, propylene glycol alginate, alginic acid, polyvinyl alcohol, povidone (PVP), carbomer, potassium pectate, potassium pectinate, and protein derived materials. Of these polymers, the cellulose ethers, especially hydroxyalkyl celluloses and carboxyalkyl celluloses are preferred. The oral dosage form may contain between 10% and 80% w/w, preferably from 20% to 70%, more preferably from 30% to 60%, of at least one hydrophilic polymer.
(b) Hydrophobic polymers include but are not limited to ethyl cellulose, hydroxyethylcellulose, hydrophobic acrylic polymers and polyvinylacetate (PVAc) based polymers. Other hydrophobic materials which may be employ are digestible, long chain (C8C50, especially C12-C40), substituted or unsubstituted hydrocarbons, such as fatty acids, fatty alcohols such as cetostearyl alcohol, stearyl alcohol; cetyl alcohol myristyl alcohol etc, fatty acids, mineral and vegetable oils and waxes such as beeswax, carnauba wax, microcrystalline wax, and ozokerite. Hydrocarbons having a melting point of between 25° and 90° C. are preferred. Of these long chain hydrocarbon materials, fatty (aliphatic) alcohols are preferred. The oral dosage form may contain up to 55-60% w/w of at least one digestible, long chain hydrocarbon.
(c) Polyalkylene glycols. The oral dosage form may contain up to 60% w/w of at least one polyalkylene glycol.

In some embodiments, the dienogest: matrix forming agent weight ratio may be about 1:2.5, about 1:5, about 1:10, about 1:12.5, about 1:15, about 1:20, about 1:25, about 1:30, about 1:35, about 1:40, about 1:45 or about 1:50. In preferred embodiments, is of 1:20 to 1:30, such as about 1:20, about 1:21, about 1:22, about 1:23, about 1:24, about 1:25, about 1:26, about 1:27, about 1:28, about 1:29 or about 1:30.

The at least one hydroxyalkyl cellulose is preferably a hydroxy (C1 to C6) alkyl cellulose, preferably selected from hydroxypropylcellulose, hydroxypropylmethylcellulose and hydroxyethyl cellulose, more preferably said hydrophilic polymer is HMPC. Preferably, HPMC in controlled-release (CR) grade. Typical HPMC products used in controlled release matrices are METHOCEL (HPMC) K100 Premium LV, K4M Premium, K15M Premium, K100M Premium, E4M Premium, and E10M Premium CR. All of these products are available in controlled-release (CR) grades, which are specially produced, ultra-fine particle size materials. These grades differ primarily in viscosity and methoxyl substitution type.

The viscosity of the matrix forming agents (e.g. HPMC) may be of 2 to 150,000 mPa·s in a 2% by weight aqueous solution at 20° C. (determined using a Pharm. Eu. capillary viscosimeter). In a particular embodiment, said composition comprises as polymeric matrix forming agent, a HPMC with a viscosity of 80000-120000 cP, such as HPMC K100M. In a preferred embodiment the composition comprises as matrix forming agent a HPMC with a viscosity of 80-120 mPa-s, such as HPMC K100 Premium LV. The viscosity ranges provided herein correspond to apparent viscosity at 2% in water at 20° C. (determined using a Pharm. Eu. capillary viscosimeter).

The amount of the at least one hydroxyalkyl cellulose in the present oral dosage form will be determined, inter alia, by the precise rate of EE, and preferably EE and DNG, release required.

In preferred embodiments, said hydrophilic polymer, preferably a hydroxy (C1 to C6) alkyl cellulose, or the preferred embodiments described herein above, is at a concentration from 25% to 60% w/w, such as from 30% to 50% w/w, including about 30%, about 40% and about 50%, or from 45% to 55% w/w, more preferably around 50% w/w.

Acrylic polymers such as polymethacrylates are pharmacologically inactive and have good compatibility with the skin and mucosal membranes. Eudragit is a trade name of copolymers derived from esters of acrylic and methacrylic acids. Eudragit grades differ in their proportion of neutral, alkaline or acid groups resulting in different physicochemical properties. In preferred embodiments of the present invention, the gastroinsoluble polymethacrylate grade (Eudragit RL/RS) is preferred. More preferably, this acrylic resin is Eudragit RS PO.

The amount of the at least one acrylic polymer in the present oral dosage form will be determined, inter alia, by the precise rate of EE, and preferably EE and DNG, release required. In preferred embodiments, said acrylic resin is found at 10% w/w to 40% w/w, preferably at 20% w/w to 30% w/w.

In some embodiments, said matrix composition further contains a glidant. A variety of agents may be incorporated as glidant agent (e.g., fumed silicon dioxides, Aerosil™, Aerosil™ C0K84, Aerosil™ 200, etc.). Glidants enhance the pharmaceutical formulations of the invention by increasing the viscosity of solutions complementing the action of cellulose ethers (e.g., HPMCs).

In addition to the above ingredients, a sustained-release matrix may also contain suitable quantities of other excipients, e.g., diluents, lubricants, binders, granulating aids, colorants, flavorants and glidants that are conventional in the pharmaceutical art.

Suitable diluents, also known as fillers, include corn starch, microcrystalline cellulose, powdered cellulose, silicified cellulose, lactose monohydrate, anhydrous lactose, mannitol, sorbitol, sucrose, fructose, dextrose, and/or mixtures thereof. Preferably, lactose monohydrate and mannitol are used. Diluents may be presents in an amount from about 20% to about 95% by weight, preferably from 30% to 90% by weight, and more preferably from 35% to 80% by weight, even more preferably from 30% to 60%, including about 40%, about 45%, about 50%, about 55% and about 60% by weight of the total weight of the composition.

The dosage form according to the invention may also comprise a binder. The binding agent can be selected from the group consisting of hydroxypropyl cellulose, hydroxypropyl methyl cellulose, hydroxyethyl cellulose, methylcellulose, polyvinylpyrrolidone, sodium carboxymethyl cellulose, calcium carboxymethyl cellulose, and/or mixtures thereof. Preferably polyvinylpyrrolidone (e.g. Povidone K30) is used. Binders may be present in an amount from about 0.5% to about 20% by weight, preferably from 1% to 10% by weight, and more preferably from 2-7% by weight, preferably about 5% by weight of the total weight of the composition.

The dosage form according to the invention can also comprise a disintegration agent. Disintegrating agents may be selected from the group consisting of low-substituted hydroxypropyl cellulose, sodium carboxymethyl cellulose, calcium carboxymethyl cellulose, crospovidone, sodium croscarmellose, and/or mixtures thereof. Disintegrating agents may be present in an amount from about 2% to about 50% by weight, preferably from about 5% to about 45% by weight, and more preferably from 10% to 40% by weight of the total weight of the composition.

Lubricants may be selected from the group consisting of talc, alkaline earth salts of stearic acid, such as magnesium and calcium stearate, stearic acid, glycerin palmitostearate, stearyl fumarate, and/or mixtures thereof. In preferred embodiments, the lubricant is magnesium stearate. The lubricant may be present in an amount from about 0% to 5% by weight, preferably from about 1% to about 3% (e.g., about 2%) based of the total weight of the composition.

In preferred embodiments, the extended-release oral dosage form of the invention comprises:
a diluent, preferably at 30-60% w/w;
a polymeric matrix forming agent, preferably at 10-60% w/w;
a binder, preferably at 1-10% w/w; and
a lubricant, preferably at 0-5% w/w.

In further preferred embodiments, said composition comprises:
lactose (e.g. lactose monohydrate) at 35-45% w/w; preferably around 40% w/w;
HPMC (e.g. HPMC K100) at 45-55% w/w, preferably around 50% w/w;
povidone (e.g. povidone K30) at 2.5-7.5% w/w, preferably around 5% w/w; and
magnesium stearate at 1.5-2.5% w/w, preferably around 2% w/w.

In even preferred embodiments, said composition comprises:
lactose monohydrate around 40% w/w;
HPMC K100 around 50% w/w;
povidone K30 around 5% w/w; and
magnesium stearate around 2% w/w.

In some embodiments the polymeric matrix is a hydroxypropyl methylcellulose (HPMC). In some embodiments the polymeric matrix is HPMC K100 (a hydroxypropyl methylcellulose thickener yielding a viscosity of 100,000 cP at 2% in water).

In some embodiments the diluent is a lactose. In some embodiments the diluent is lactose monohydrate.

In some embodiments the extended-release matrix comprises: (a) 30-60% w/w of a diluent based on a total weight of the composition; (b) 10-60% w/w of a polymeric matrix based on the total weight of the composition; (c) 1-10% w/w of a binder based on a total weight of the composition; and (d) 0-5% w/w of a lubricant based on a total weight of the composition.

In some embodiments the extended-release matrix comprises: (a) 35-45% w/w of a lactose based on a total weight of the composition; (b) 45-55% w/w of a hydroxypropyl methylcellulose (HPMC) based on the total weight of the composition; (c) 2-7% w/w of a povidone based on the total weight of the composition; and (d) 1-3% w/w of a stearate based on the total weight of the composition.

In some embodiments the extended-release matrix comprises: (a) 35-45% w/w of lactose monohydrate based on a total weight of the composition; (b) 45-55% w/w of HPMC K100 (a hydroxypropyl methylcellulose thickener yielding a viscosity of 100,000 cP at 2% in water) based on the total weight of the composition; (c) 2-7% w/w of povidone K30 (a cross-linked polyvinylpyrrolidone having average molecular weight of 40,000) based on the total weight of the composition; and (d) 1-3% w/w of a magnesium stearate based on the total weight of the composition.

In some embodiments the extended-release matrix comprises: (a) 46.82% w/w of the HPMC K100 (a hydroxypropyl methylcellulose thickener yielding a viscosity of 100,000 cP at 2% in water) based on a total weight of the composition; (b) 36.48% w/w of the lactose monohydrate based on the total weight of the composition; (c) 4.68% w/w of the povidone K30 (a cross-linked polyvinylpyrrolidone having average molecular weight of 40,000) based on a total weight of the composition; and (d) 1.87% w/w of the magnesium stearate based on a total weight of the composition.

In some embodiments the extended-release matrix has a Carr Index ranging from 17% to 18%. In some embodiments the extended-release matrix has a Carr Index ranging from 17.0% to 17.5%.

In some embodiments the oral contraceptive composition is in the form of a tablet comprising the extended-release matrix. In some embodiments a hardness of the tablet ranges from 15N to 60 N. In some embodiments a hardness of the tablet ranges from 28N to 58 N. In some embodiments n the tablet is a film-coated tablet comprising a tablet core and a film coating, and wherein the tablet core comprises the extended-release matrix. In some embodiments the film coating comprises a polymer, a plasticizer and a pigment. In some embodiments the polymer comprises a polyethylene glycol. In some embodiments the polymer comprises a polyethylene glycol having an average molecular weight within 5,000-7,000.

In some embodiments the oral contraceptive composition does not comprise estrogen.

In order to facilitate the preparation of an extended-release oral dosage form according to this invention there is provided, another aspect of the present invention, a process for the preparation of an extended-release oral dosage form according to the present invention. In preferred embodiments, said dosage oral form is a solid form (e.g. tablets or capsules).

Methods for the manufacturing of the extended-release oral dosage form of the invention are well known in the art, such as wet granulation, dry granulation or direct compression. In wet granulation, components are typically mixed and granulated using a wet binder. The wet granulates are then sieved, dried and optionally ground prior to compressing into tablets. Dry granulation is usually described as a method of controlled crushing of precompacted powders densified by either slugging or passing the material between two counter-rotating rolls. More specifically, powdered components that may contain very fine particles are typically mixed prior to being compacted to yield hard slugs which are then ground and sieved before the addition of other ingredients and final compression to form tablets. Direct compression is generally considered to be the simplest and the most economical process for producing tablets. However, it may only be applied to materials that don't need to be granulated before tableting. Direct compression requires only two principal steps; i.e., the mixing of all the ingredients and the compression of this mixture. However, direct compression is applicable to only a relatively small number of substances as the ingredients of the tablets often need to be processed by some granulation technique to make them compressible and/or for improving their homogeneity and flowability.

Mixing and formulation of low dose drugs can be very challenging due to problems related to segregation, content uniformity and physical stability. A careful control on these factors may be necessary while manufacturing the oral dosage form described herein. Many types of equipments have been designed to facilitate mixing of low dose drugs with excipients which could also be used in the manufacturing of the extended-release oral form of the invention, including but not limited to high shear granulation, ordered mixing and spray drying. Remington: The Science and Practice of Pharmacy, Pharmaceutical Press, 2013.

In a particular embodiment, said process is for obtaining a solid, extended-release oral dosage form and comprises incorporating EE, preferably EE and DNG, in a extended-release matrix. This process may comprise the following steps:
i. mixing EE and optionally DNG, with a diluent, and then mixing these together to obtain a mixture;
ii. granulating a diluent and a polymeric matrix forming agent as described herein;
iii. drying, optionally sieving, and mixing the granules obtained in step ii) with the mixture obtained in step i).

In some embodiments the extended-release matrix comprises a diluent, a polymeric matrix, a binder, and a lubricant. In some embodiments the extended-release matrix is formed from a granular core comprising a polymeric matrix forming agent and a diluent. In some embodiments the extended-release matrix comprises the granular core, the DNG, the EE, and an additional amount of the diluent. A mass ratio of the polymeric matrix forming agent to the diluent in the granular core may range from 2:1 to 10:1. For example, in some embodiments the mass ratio of the polymeric matrix forming agent to the diluent in the granular core ranges from 3:1 to 10:1, or from 3:1 to 9:1, or from 4:1 to 9:1, or from 4:1 to 8:1, or from 5:1 to 8:1, or from 5.5:1 to 8.5:1, or from 6:1 to 8:1, or from 6.5 to 8:1, or from 6.5:1 to 7:1, or from 7:1 to 8:1, or from 7:1 to 7.5:1, or from 7.5:1 to 8:1, or from 8:1 to 8.5:1. In some embodiments the mass ratio of the polymeric matrix forming agent to the diluent in the granular core is about 7.2:1. In some embodiments the granular core further comprises a binder.

In preferred embodiments, said oral dosage form is a tablet and the process further comprises:
iv. blending the mixture obtained in step iii) with a lubricant, and then compress into tablets;
v. optionally coating the tablets obtained in step iv) with a coating agent and dry.

Alternatively, the mixture obtained in step iii) can be used to fill hard gelatin capsules.

In another embodiment, when only there is a modified or extended release of EE, a multiparticulate dose formulation as described above (e.g. pellets) may be preferred. Said process may comprise:
a. mixing together DNG immediate release and EE extended-release fraction; and
b. fill in hard gelatin capsules or compress in tablets (e.g., see steps iv and v above).

The contraceptive composition as described herein may be suitable for administration as the daily active oral form in various administration regimens, preferred administration regimens are described herein below for contraceptive purposes but may also be used for medical purposes referred herein.

In a particular embodiment, said composition is suitable for administration as the daily active oral form in a regimen comprising the administration of the active oral form for 24 consecutive days followed by a period of 4 days of daily administration of a placebo oral form. In another embodiment, said composition is suitable for administration as the daily active oral form in a regimen comprising the administration of the active oral form daily for 87 consecutive days followed by a period of 4 days of daily administration of a placebo oral form or no oral form administration.

### Medical Uses and Methods of the Composition

In a further aspect, the present invention also provides an oral composition as described herein for use in the treatment of one or more of the following diseases or disorders in a female subject, preferably a premenopausal female.
Acne
Endometriosis
Dysmenorrhea
Other: dysfunctional uterine bleeding, cycle dependent complains, uterine fibroids.

In a related aspect, it refers to a method of treating one or more of the diseases or disorders mentioned above, wherein said method comprises administering to a female subject in need thereof, preferably a premenopausal female, a therapeutically effective dosage of an oral composition as described herein.

In a further related aspect, the present invention provides the use of an oral composition as described herein for the manufacturing of a medicament for the treatment of one or more of the diseases or disorders mentioned above.

Preferred embodiments and features of the kit are as described herein above for the oral composition, contraceptive methods and uses. In particular, for endometriosis treatment an extended regimen with hormone free interval every 6 months or one year may be preferred.

### Kit of the Invention

In an additional aspect, the present invention also provides a kit based on the compositions described in the present application. Such a kit is particularly suitable for use in the contraceptive and medical methods as described above.

The said contraceptive kit comprises one or more packaging units. One or more packaging units includes, without being limited to, 1 packaging unit, 2 packaging units, 3 packaging units, 4 packaging units, 5 packaging units and 6 packaging units.

Each packaging unit may comprise from 21 to 28 daily active dosage units. As fully described above, each daily active dosage unit consists of a composition as described herein.

In some embodiments, the contraceptive kit is characterized in that each packaging unit comprises 28 daily dosage units and no daily dosage unit of a pharmaceutically acceptable placebo. Such a contraceptive kit is particularly appropriate to perform the contraceptive method of the invention which consists in administering “continuously” without free-contraceptive period.

In other embodiments each packaging unit comprises:
21 to 27 active daily dosage units consisting of a contraceptive composition as fully described in the present application; and
optionally, 1 to 7 daily dosage units of a pharmaceutically acceptable placebo.

Such a contraceptive kit is particularly appropriate to perform the contraceptive method of the invention which comprises:
a first phase wherein active daily dosage units of the invention which do not comprise estrogen are administered to the female patient over a period of 21 to 27 consecutive days followed by
a second phase wherein no contraceptive composition is administered to the female patient over a period of 1 to 7 consecutive days.

In some other embodiments, each packaging unit of the kit comprises 24 daily dosage units comprising an effective amount of a contraceptive composition as described herein and, optionally, 4 daily dosage units of a pharmaceutically acceptable placebo.

The packaging unit as described above may have one of the conventional forms usually used for oral contraceptives. For example, the packaging unit may be a conventional blister pack comprising the appropriate number of dosage units in a sealed blister pack (e.g. an aluminium blister) with a cardboard, paperboard, foil or plastic backing and enclosed in a suitable cover.

Each blister container may be conveniently numbered or marked in order to facilitate compliance. he packaging unit may contain daily dosage units in the order in which they are to be taken, i.e. starting with the first of the at least 21 dosage units that contain the combination of drospirenone optionally followed by 7 or less empty blisters or by 7 or less dosage units that comprise a pharmaceutically acceptable placebo.

The kit of the invention may comprise other appropriate components such as instructions for use.

Preferred embodiments and features of the kit are as described herein above for the oral composition, contraceptive methods and uses; and therapeutic methods and uses.

It is contemplated that any features described herein can optionally be combined with any of the embodiments of any medical or contraceptive use, composition, kit, contraceptive methods, methods of treatment, or method of manufacturing of the invention; and any embodiment discussed in this specification can be implemented with respect to any of these. It will be understood that certain embodiments described herein are shown by way of illustration and not as limitations of the invention.

All publications and patent applications are herein incorporated by reference to the same extent as if each individual publication or patent application was specifically and individually indicated to be incorporated by reference.

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “comprises” also encompasses and expressly discloses the terms “consists of” and “consists essentially of”. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination unless otherwise apparent from the context.

As used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by ±1, 2, 3, 4, 5, 6, 7, 8, 9, or 10%. Accordingly, the term “about” may mean the indicated value ±5% of its value, preferably the indicated value ±2% of its value, most preferably the term “about” means exactly the indicated value (±0%).

The following examples serve to illustrate the present invention and should not be construed as limiting the scope thereof.

### EXAMPLES

### A. Preparation and Dissolution Profiles of DNG-EE Extended-Release Formulations

### Example 1: Dissolution Profiles of DNG-EE Extended-Release Formulations with Alternative Matrix Forming Polymers at Various Concentrations

### Lactose—Eudragit RS PO: Formulation 1

Table 1 shows the qualitative and quantitative composition of Formulation 1.

TABLE 1
Composition of Formulation 1

Formulation 1

| Material | mg/tablet | % (w/w) |
|---|---|---|
| Dienogest | 2.00 | 4.00 |
| Ethinyl estradiol | 0.02 | 0.04 |
| Lactose | 35.48 | 70.96 |
| monohydrate | | |
| Eudragit RS PO | 10.00 | 20.00 |
| Povidone K30 | 1.50 | 3.00 |
| Ethanol 96% | * | |
| Water | * | |
| Magnesium | 1.00 | 2.00 |
| stearate | | |
| Total | 50.0 | 100 |

* Evaporated during the process

### Manufacturing Process:

Eudragit RS PO and lactose (1^{st} fraction, 16 mg) were granulated with the binder solution: Water, Ethanol 96%, Povidone K30. The granules obtained were mixed and blended with dienogest, Ethinyl estradiol and the remaining lactose (2^{nd} fraction, remaining lactose). The granules were lubricated with Magnesium stearate and compressed to round, biconvex tablets, diameter 5.0 mm, average weight 50.0 mg.

The “Dissolution Method I” used for testing dissolution of the Dienogest/Ethinyl Estradiol 2.00 mg/0.02 mg extended-release tablets is shown in Table 2 below.

TABLE 2
Dissolution Method I

| Apparatus: | USP 1 (baskets) | |
|---|---|---|
| Medium: | Water, purified | |
| Speed: | 75 | rpm |
| Volume: | 900 | mL |

| | |
|---|---|
| Temperature: | 37° C. ± 0.5° C. |

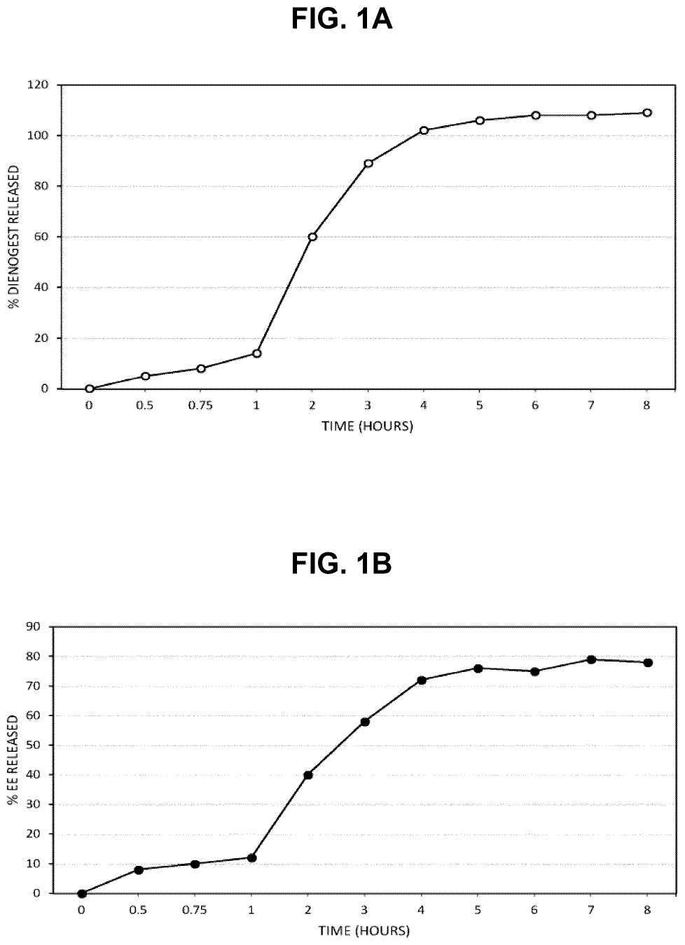

The dissolution profile of dienogest and EE in Formulation 1 is shown in FIGS. 1A and 1B, respectively.

### Mannitol—Eudragit RS PO: Formulations 2 and 3

Table 3 below shows the qualitative and quantitative composition of Formulations 2 and 3. Formulations 2 and 3 show the effect of Eudragit RS PO at concentrations of 30% and 40% in the dissolution profile of both dienogest and ethinyl estradiol. The quantity of Eudragit RS PO was adjusted by changing the quantity of Mannitol.

TABLE 3
Composition of Formulations 2 and 3

| | Formulation 2 | | Formulation 3 | |
|---|---|---|---|---|
| Material | mg/tablet | % (w/w) | mg/tablet | % (w/w) |
| Dienogest | 2.00 | 4.00 | 2.00 | 4.00 |
| Ethinyl estradiol | 0.02 | 0.04 | 0.02 | 0.04 |
| Mannitol | 29.48 | 58.96 | 24.48 | 48.96 |
| Eudragit RS PO | 15.00 | 30.00 | 20.00 | 40.00 |
| Povidone K30 | 2.50 | 5.00 | 2.50 | 5.00 |
| Ethanol 96% | * | * | * | * |
| Water | * | * | * | * |
| Magnesium stearate | 1.00 | 2.00 | 1.00 | 2.00 |
| Total | 50.0 | 100 | 50.0 | 100 |

* Evaporated during the process

### Manufacturing Process:

Eudragit RS PO and mannitol (1st fraction, 16 mg) were granulated with the binder solution: Water, Ethanol 96%, Povidone K30. The granules obtained were mixed and blended with dienogest, Ethinyl estradiol and the remaining lactose (2^{nd} fraction). The granules were lubricated with Magnesium stearate and compressed to round, biconvex tablets, diameter 5.0 mm, average weight 50.0 mg.

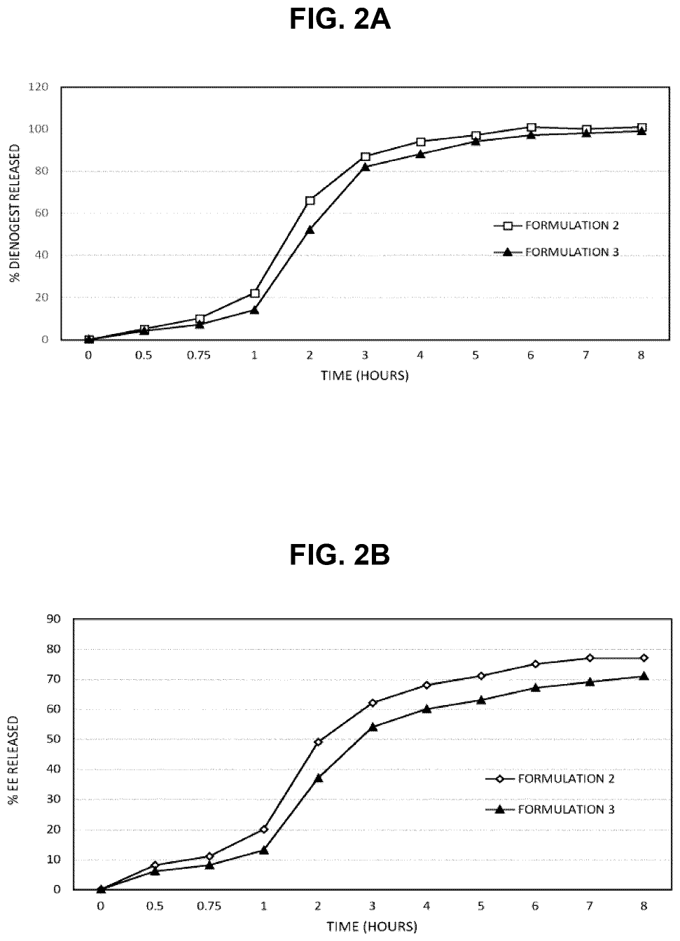

The dissolution profiles of Formulations 2 and 3 were obtained using the Dissolution Method I described in Table 2. The dissolution profile of dienogest and EE in Formulations 2 and 3 is shown in FIGS. 2A and 2B, respectively.

### Lactose—HPMC: Formulations 4 and 5

Table 4 below shows the qualitative and quantitative composition of Formulations 4 and 5. Formulations 4 and 5 show the effect of HPMC K100M at concentrations of about 30% and about 50% in the dissolution profile of both dienogest and ethinyl estradiol. The quantity of HPMC was adjusted by changing the quantity of lactose. Table 9 shows the qualitative and quantitative composition of Formulations 4 and 5.

TABLE 4
Composition of Formulations 4 and 5

| | Formulation 4 | | Formulation 5 | |
|---|---|---|---|---|
| Material | mg/tablet | % (w/w) | mg/tablet | % (w/w) |

CORE

| | | | | |
|---|---|---|---|---|
| Dienogest | 2.00 | 3.75 | 2.00 | 3.75 |
| Ethinyl estradiol | 0.02 | 0.04 | 0.02 | 0.04 |
| Lactose | 29.48 | 55.20 | 19.48 | 36.48 |
| HPMC K100M | 15.00 | 28.10 | 25.00 | 46.82 |
| Povidone K30 | 2.50 | 4.68 | 2.50 | 4.68 |
| Ethanol 96% | * | * | * | * |
| Water | * | * | * | * |
| Magnesium stearate | 1.00 | 1.87 | 1.00 | 1.87 |

COATING

| | | | | |
|---|---|---|---|---|
| Opadry 2 white | 1.00 | 1.87 | 1.00 | 1.87 |
| PEG 6000 | 2.40 | 4.49 | 2.40 | 4.49 |
| Total | 50.0 | 100 | 50.0 | 100 |

* Evaporated during the process

### Manufacturing Process:

HPMC and lactose (1^{st} fraction, 16 mg) were granulated with the binder solution: Water, Ethanol 96%, Povidone K30. The granules obtained were mixed and blended with dienogest, Ethinyl estradiol and the remaining lactose (2^{nd} fraction). The granules were lubricated with Magnesium stearate and compressed to round, biconvex tablets, diameter 5.0 mm, average weight 50.0 mg. Film coating of tablet cores with white Opadry and PEG6000 in film coater HT0003.

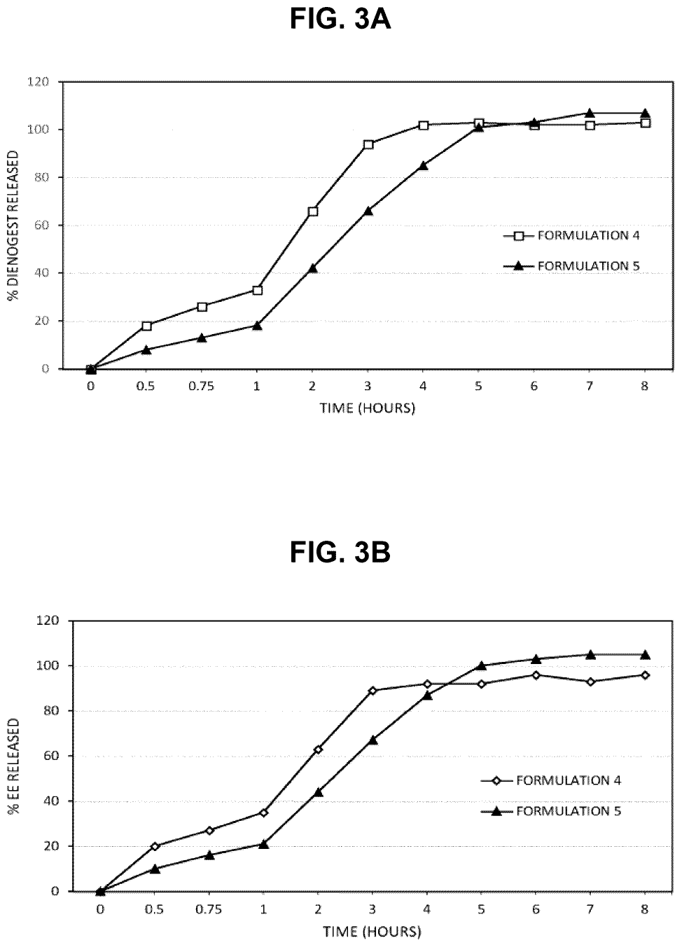

The dissolution profiles of Formulations 4 and 5 were obtained using the Dissolution Method I described in Table 2. The dissolution profile of dienogest and EE in Formulations 4 and 5 is shown in FIGS. 3A and 3B, respectively.

Formulation 5 with 50% HPMC (i.e., 46.82% of HPMC) presents the preferred prolonged-release profile.

### Lactose—HPMC: Formulations 6, 7 and 8

Table 5 below provides the qualitative and quantitative composition of Formulations 6 to 8, comprising 1 mg DNG/0.01 mg EE, 2 mg DNG/0.01 mg EE, and 2 mg DNG/0.02 mg, respectively. These formulations are lactose-HPMC formulations with about 50% HPMC K100M produced by the same manufacturing process used in Formulations 4 and 5 above.

TABLE 5
Composition of Formulations 6, 7 and 8

| | Formulation 6 | | Formulation 7 | | Formulation 8 | |
|---|---|---|---|---|---|---|
| Material | mg/tablet | % (w/w) | mg/tablet | % (w/w) | mg/tablet | % (w/w) |

CORE

| | | | | | | |
|---|---|---|---|---|---|---|
| Dienogest | 1.00 | 1.87 | 2.00 | 3.75 | 2.00 | 3.75 |
| Ethinyl | 0.01 | 0.02 | 0.01 | 0.02 | 0.02 | 0.04 |
| estradiol | | | | | | |
| Lactose | 20.49 | 38.37 | 19.48 | 36.50 | 19.48 | 36.48 |
| monhydrate | | | | | | |
| HPMC K100M | 25.00 | 46.82 | 25.00 | 46.82 | 25.00 | 46.82 |
| Povidone K30 | 2.50 | 4.68 | 2.50 | 4.68 | 2.50 | 4.68 |
| Ethanol 96% | * | * | * | * | * | * |
| Water | * | * | * | * | * | * |
| Magnesium | 1.00 | 1.87 | 1.00 | 1.87 | 1.00 | 1.87 |
| stearate | | | | | | |

COATING

| | | | | | | |
|---|---|---|---|---|---|---|
| Opadry 2 yelow | 1.00 | 1.87 | — | — | — | — |
| Opadry 2 pink | — | — | 1.00 | 1.87 | — | — |
| Opadry 2 white | — | — | — | — | 1.00 | 1.87 |
| PEG 6000 | 2.40 | 4.49 | 2.40 | 4.49 | 2.40 | 4.49 |
| Total | 53.4 | 100 | 53.4 | 100 | 53.4 | 100 |

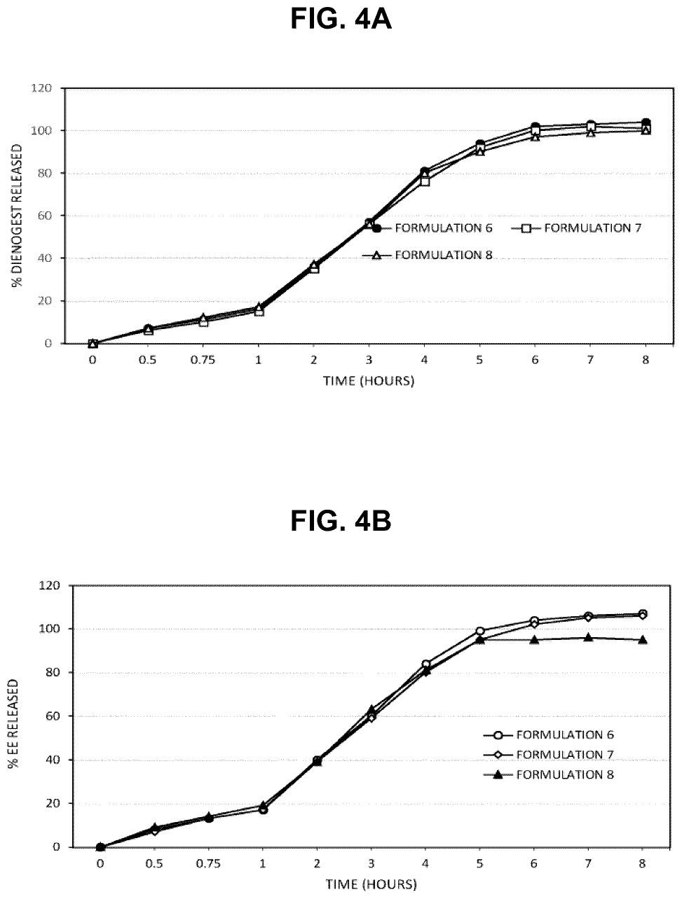

The dissolution profiles of Formulations 6, 7 and 8 were obtained using the Dissolution Method I described in Table 2. The dissolution profile of dienogest and EE in formulations 6 to 8 are shown in FIGS. 4A and 4B, respectively. It can be seen that the various batches produced present almost overlapping dissolution profiles.

### Example 2: Effect on Granulation Parameters on Characteristics of Lactose-HPMC (DNG 2 Mg EE 0.02 mg) Extended-Release Formulations

### Manufacturing Process:

A two-step wet granulation process using a high shear mixer was carried out to prepare various Lactose-HPMC EE 0.02 mg/DNG 2 mg extended-release formulations.

Step 1: These formulations were prepared as prolonged-release tablets where lactose monohydrate (1^{st} fraction) and HPMC are first pre-blended in the bin blender. This pre-blend was granulated in a high shear mixer with a granulation solution prepared by dissolving of povidone in ethanol and purified water and sprayed on the bottom of the granulator to obtain dried pre-blend granules. The dried pre-blend granules were then passed through a mesh for sizing.

Step 2: The dried and sized pre-blend granules were mixed in a bin blender with Dienogest (DNG), Ethinylestradiol (EE) and lactose monohydrate (2^{nd} fraction). The resulting blend was lubricated in a bin blender with Magnesium Stearate. The final blend was compressed using a rotary tablet press into tablets as per set specifications and finally coated in a coating pan.

Evaluation of certain process variables that can potentially impact on the quality attributes of the Lactose-HPMC (DNG 2 mg EE 0.02 mg) extended-release formulations was carried out as described below.
Effect of Mesh Size: Formulations 9, 10 and 11

Three types of different sieve mesh sizes, and their influence on the PSD size and granule flow character, were tested for the extended-release Formulation 8 described above. Table 6 below shows the data obtained in the different batches sieved respectively through 0.8 mm, 1.5 mm and 2.0 mm.

TABLE 6
Effect of different sieve mesh size on PSD and flow characters of the granules

| | 0.8 mm mesh size | | 1.5 mm mesh size | | 2.0 mm mesh size | |
|---|---|---|---|---|---|---|
| Sample | Formulation 9 | | Formulation 10 | | Formulation 11 | |
| Sieve | % | % | % | % | % | % |
| 710 μm | 0.14 | 0.15 | 10.28 | 8.58 | 10.41 | 12.90 |
| 500 μm | 7.85 | 0.64 | 9.53 | 11.76 | 7.15 | 4.68 |
| 355 μm | 9.44 | 4.51 | 5.96 | 5.89 | 4.95 | 3.10 |
| 250 μm | 4.71 | 5.00 | 4.56 | 4.82 | 3.15 | 1.67 |
| 180 μm | 3.99 | 8.84 | 4.66 | 5.11 | 3.13 | 1.81 |
| 125 μm | 6.20 | 6.36 | 13.59 | 9.05 | 7.61 | 5.00 |
| 90 μm | 12.74 | 10.06 | 17.16 | 14.41 | 1.47 | 8.89 |
| <90 μm | 54.77 | 64.39 | 34.27 | 40.38 | 62.09 | 61.85 |
| Total | 99.84 | 99.95 | 100.01 | 100.00 | 99.96 | 99.90 |
| Bulk density (g/ml) | 0.39 | 0.36 | 0.47 | 2.46 | 0.40 | 0.39 |
| Tapped Density | 0.56 | 0.53 | 0.57 | 0.57 | 0.55 | 0.58 |
| (g/ml) | | | | | | |
| Carr Index % | 30.36 | 32.08 | 17.19 | 17.18 | 27.00 | 33.84 |
| Flow character | POOR | VERY | FAIR | FAIR | POOR | VERY |
| | | POOR | | | | POOR |

The comparison of the batches shows that the granules sieved through 1.5 mm (Cl 17.18-17.19%) present a better flow of granules than those sieved through 0.8 mm and 2.0 mm (Cl 30.36-32.08% vs 27.00-33.84%) due to granules PSD increase. The granules of Formulation 10 milled through Conical mill at 1.5 mm produced consistent particle size distribution and free flowing granules to ensure consistent particle size distribution on milled granules on every batch.
Effect of Compression Force/Hardness: Formulations 12, 13 and 14

The effect of main compression force during tableting of the extended-release Formulation 8 described above was evaluated. A batch size of 60,000 was tableted at different compression forces (from 15N to 50N) to evaluate the impact on dissolution. A pilot compression machine Kilian S100MX (power assisted, rotary tablet press) was employed, the process parameters are summarized in Table 7 below:

TABLE 7
Tableting parameters

| PROCESS | | | |
|---|---|---|---|
| PARAMETERS | Formulation 12 | Formulation 13 | Formulation 14 |
| Hardness (N) | 15 | 25 | 50 |
| Compression height (mm) | 1.0 | 0.5 | 0.2 |
| Compression speed (tabs/h) | 25,000 | 25,000 | 25,000 |

The dissolution profiles of Formulations 12-14 were then obtained using the “Dissolution Method II” summarized below in Table 8.

TABLE 8
Dissolution Method II

| | |
|---|---|
| Dissolution Medium | Water, 500 ml |
| Apparatus | USP I Baskets |
| Speed rotation | 75 rpm |
| Temperature | 37.0 ± 0.5° C. |
| Timepoints | 30, 45, 60, 120, 180, 240, 300, 360, |
| | 420, 480 and infinite point at 200 rpm |

The dissolution profile of DNG for Formulations 12-14 is summarized in Table 9 below.

TABLE 9
DNG dissolution profiles from film-coated tablets
at different hardness and compression

| | Formulation 12 | | Formulation 13 | | Formulation 14 | |
|---|---|---|---|---|---|---|
| Time | (15N) | | (25N) | | (50N) | |
| (min) | DNG Mean (%) | RSD | DNG Mean (%) | RSD | DNG Mean (%) | RSD |
| 0 | 0 | 0 | 0 | 0 | 0 | 0 |
| 30 | 9 | 9.8 | 12 | 8.7 | 7 | 17.9 |
| 45 | 16 | 12.4 | 19 | 8.4 | 12 | 23.6 |
| 60 | 21 | 11.8 | 24 | 8.1 | 17 | 26.1 |
| 120 | 48 | 12.4 | 55 | 6.7 | 42 | 8.5 |
| 180 | 69 | 6.7 | 79 | 6.7 | 65 | 6.2 |
| 240 | 84 | 2.1 | 94 | 4.3 | 81 | 5.9 |
| 300 | 92 | 1.1 | 103 | 3.1 | 91 | 5.0 |
| 360 | 94 | 2.0 | 106 | 3.3 | 96 | 4.3 |
| 420 | 94 | 1.7 | 109 | 5.1 | 99 | 3.6 |
| 480 | 94 | 1.8 | 107 | 3.4 | 100 | 3.2 |
| inf | 94 | 1.8 | 108 | 3.6 | 77 | 7.6 |

The dissolution profile of EE for Formulations 12-14 is summarized in Table 10 below.

TABLE 10
EE dissolution profiles for film-coated tablets
at different hardness and compression

| | Formulation 12 | | Formulation 13 | | Formulation 14 | |
|---|---|---|---|---|---|---|
| Time | (15N) | | (25N) | | (50N) | |
| (min) | EE Mean (%) | RSD | EE Mean (%) | RSD | EE Mean (%) | RSD |
| 0 | 0 | 0 | 0 | 0 | 0 | 0 |
| 30 | 13 | 9.7 | 15 | 8.8 | 10 | 19.8 |
| 45 | 19 | 8.9 | 22 | 13.3 | 14 | 10.9 |
| 60 | 24 | 7.9 | 27 | 13.5 | 20 | 19.5 |
| 120 | 51 | 11.0 | 58 | 7.4 | 45 | 9.6 |
| 180 | 74 | 6.6 | 82 | 8.0 | 72 | 5.8 |
| 240 | 91 | 3.5 | 99 | 3.7 | 89 | 4.8 |
| 300 | 98 | 2.4 | 106 | 3.5 | 100 | 4.6 |
| 360 | 97 | 3.5 | 108 | 4.6 | 105 | 2.5 |
| 420 | 97 | 3.8 | 110 | 6.3 | 104 | 3.7 |
| 480 | 96 | 3.8 | 109 | 3.8 | 103 | 2.9 |
| inf | 96 | 4.1 | 108 | 3.9 | 88 | 7.3 |

Effect of HPMC/Lactose Mass Ratio of Pre-Blend Granules: Formulations 15, 16 and 17

The effect of HPMC/Lactose mass ratio within the pre-blend granules formed in Step 1 of the two-step wet granulation process, described above, was studied in Formulations 15, 16 and 17 summarized in Table 11 below.

TABLE 11
Composition of Formulations 15, 16 and 17

| BATCH | Formulation 15 | | Formulation 16 | | Formulation 17 | |
|---|---|---|---|---|---|---|
| MATERIAL | mg/Tablet | Formula % | mg/Tablet | Formula % | mg/Tablet | Formula % |

PREBLEND GEOMETRICAL DILUTION

| | | | | | | |
|---|---|---|---|---|---|---|
| DIENOGEST | 2.00 | 3.75 | 2.00 | 3.75 | 2.00 | 3.75 |
| ETHINYL | 0.02 | 0.04 | 0.02 | 0.04 | 0.02 | 0.04 |
| ESTRADIOL | | | | | | |
| LACTOSE | 16.00 | 29.96 | 16.00 | 29.96 | 16.00 | 29.96 |
| MONOHYDRATE | | | | | | |
| GRANULAC 200 | | | | | | |
| LACTOSE | — | — | — | — | — | — |
| MONOHYDRATE | | | | | | |
| FAST FLO | | | | | | |

GRANULATION

| | | | | | | |
|---|---|---|---|---|---|---|
| HPMC K100 LV CR | 5.00 | 9.36 | 15.00 | 28.10 | 25.00 | 46.82 |
| LACTOSE | 23.48 | 43.97 | 13.48 | 25.24 | 3.48 | 6.52 |
| MONOHYDRATE | | | | | | |
| GRANULAC 200 | | | | | | |
| LACTOSE | — | — | — | — | — | — |
| MONOHYDRATE | | | | | | |
| FAST FLO | | | | | | |

BINDER SOLUTION

| | | | | | | |
|---|---|---|---|---|---|---|
| POVIDONE K30 | 2.50 | 4.68 | 2.50 | 4.68 | 2.50 | 4.68 |
| ETHANOL 96% | * | * | * | * | * | * |
| WATER | * | * | * | * | * | * |

FINAL BLEND

| | | | | | | |
|---|---|---|---|---|---|---|
| MAGNESIUM | 1.00 | 1.87 | 1.00 | 1.87 | 1.00 | 1.87 |
| STEARATE | | | | | | |

COATING

| | | | | | | |
|---|---|---|---|---|---|---|
| OPADRY 2 WHITE | 1.00 | 1.87 | 1.00 | 1.87 | 1.00 | 1.87 |
| PEG 6000 | 2.40 | 4.49 | 2.40 | 4.49 | 2.40 | 4.49 |
| WATER, PURIFIED | * | * | * | * | * | * |
| TOTAL | 53.4 | 100.0 | 53.4 | 100.0 | 53.4 | 100.0 |

The dissolution profile of DNG for Formulations 15-17 prepared at different hardness levels is summarized in Table 12 below. The data in Table 12 shows that Formulation 17 exhibits an optimal DNG dissolution profile—and this is true when applying both 30N and 58N of compression force.

TABLE 12
DNG dissolution profiles for file-coating tablets prepared using
different HPMC/Lactose mass ratios within the pre-blend granules

| | | Form. 15 | | Form. 16 | | Form. 17 | | Form. 15 | | Form. 16 | | Form. 17 | |
|---|---|---|---|---|---|---|---|---|---|---|---|---|---|
| Time | Time | (30N) | | (30N) | | (30N) | | (39N) | | (63N) | | (58N) | |
| (min) | (h) | Mean (%) | RSD | Mean (%) | RSD | Mean (%) | RSD | Mean (%) | RSD | Mean (%) | RSD | Mean (%) | RSD |
| 0 | 0 | 0 | 0.0 | 0 | 0.0 | 0 | 0.0 | 0 | 0.0 | 0 | 0.0 | 0 | 0.0 |
| 30 | 0.5 | 90 | 6.6 | 18 | 11.0 | 8 | 20.2 | 68 | 27.1 | 14 | 19.7 | 8 | 19.7 |
| 45 | 0.75 | 100 | 2.6 | 26 | 10.1 | 13 | 14.8 | 91 | 15.4 | 21 | 18.3 | 12 | 15.4 |
| 60 | 1 | 101 | 2.5 | 33 | 9.5 | 18 | 12.3 | 100 | 1.8 | 30 | 17.8 | 18 | 14.7 |
| 120 | 2 | 101 | 2.3 | 66 | 8.4 | 42 | 10.8 | 101 | 1.7 | 62 | 15.0 | 39 | 12.5 |
| 180 | 3 | 101 | 2.4 | 94 | 7.6 | 66 | 10.9 | 102 | 2.1 | 94 | 11.1 | 63 | 13.5 |
| 240 | 4 | 101 | 2.6 | 102 | 1.5 | 85 | 10.9 | 102 | 2.4 | 104 | 3.9 | 83 | 13.1 |
| 300 | 5 | 102 | 2.6 | 103 | 1.8 | 101 | 3.7 | 102 | 3.8 | 101 | 6.0 | 98 | 10.3 |
| 360 | 6 | 102 | 2.3 | 102 | 1.7 | 103 | 3.8 | 101 | 2.4 | 102 | 4.1 | 104 | 5.3 |
| 420 | 7 | 101 | 2.6 | 102 | 1.7 | 107 | 1.1 | 102 | 2.5 | 106 | 5.6 | 107 | 2.5 |
| 480 | 8 | 102 | 2.6 | 103 | 1.9 | 107 | 1.4 | 102 | 2.6 | 105 | 4.4 | 108 | 3.1 |
| PI | PI | 104 | 2.4 | 106 | 1.6 | 110 | 1.3 | 103 | 2.6 | 105 | 4.5 | 110 | 2.7 |

| | | | |
|---|---|---|---|
| Visually | First minute | Second hour: | Sixth hour: the |
| | the tablet | the tablet | tablet starts to |
| | starts to | starts to | disintegrate |

| | |
|---|---|
| disintegrate | disintegrate |

The dissolution profile of EE for Formulations 15-17 prepared at different hardness levels is summarized in Table 13 below. The data in Table 13 shows that Formulation 17 exhibits an optimal EE dissolution profile—and this is true when applying both 30N and 58N of compression force.

TABLE 13
EE dissolution profiles for file-coating tablets prepared using different
HPMC/Lactose mass ratios within the pre-blend granules

| | | Form. 15 | | Form. 16 | | Form. 17 | | Form. 15 | | Form. 16 | | Form. 17 | |
|---|---|---|---|---|---|---|---|---|---|---|---|---|---|
| | | (30N) | | (30N) | | (30N) | | (39N) | | (63N) | | (58N) | |
| Time | Time | Mean | | Mean | | Mean | | Mean | | Mean | | Mean | |
| (min) | (h) | (%) | RSD | (%) | RSD | (%) | RSD | (%) | RSD | (%) | RSD | (%) | RSD |
| 0 | 0 | 0 | 0.0 | 0 | 0.0 | 0 | 0.0 | 0 | 0.0 | 0 | 0.0 | 0 | 0.0 |
| 30 | 0.5 | 83 | 7.8 | 20 | 7.0 | 10 | 18.5 | 68 | 27.1 | 16 | 16.2 | 11 | 13.5 |
| 45 | 0.75 | 93 | 6.8 | 27 | 8.0 | 16 | 12.3 | 93 | 16.6 | 25 | 13.4 | 16 | 9.2 |
| 60 | 1 | 94 | 4.4 | 35 | 8.4 | 21 | 5.3 | 103 | 0.8 | 32 | 15.2 | 21 | 12.3 |
| 120 | 2 | 96 | 3.1 | 63 | 7.3 | 44 | 7.7 | 103 | 1.7 | 65 | 11.8 | 42 | 10.2 |
| 180 | 3 | 96 | 3.5 | 89 | 8.9 | 67 | 7.8 | 104 | 1.2 | 97 | 10.7 | 65 | 11.7 |
| 240 | 4 | 96 | 2.6 | 92 | 7.7 | 87 | 8.1 | 102 | 4.8 | 106 | 6.0 | 85 | 12.2 |
| 300 | 5 | 97 | 2.9 | 92 | 9.5 | 100 | 3.4 | 105 | 2.1 | 106 | 4.2 | 97 | 10.2 |
| 360 | 6 | 94 | 4.7 | 96 | 2.0 | 103 | 1.7 | 104 | 1.7 | 105 | 5.0 | 104 | 4.9 |
| 420 | 7 | 97 | 2.0 | 93 | 11.2 | 105 | 1.6 | 104 | 2.6 | 107 | 5.0 | 107 | 2.7 |
| 480 | 8 | 97 | 3.2 | 96 | 1.9 | 105 | 1.4 | 105 | 3.1 | 106 | 5.2 | 106 | 3.1 |
| PI | PI | 100 | 2.4 | 100 | 1.3 | 108 | 2.2 | 106 | 2.0 | 106 | 4.3 | 110 | 3.1 |

| | | | |
|---|---|---|---|
| Visually | First minute | Second hour: | Sixth hour: the |
| | the tablet | the tablet | tablet starts to |
| | starts to | starts to | disintegrate |

| | |
|---|---|
| disintegrate | disintegrate |

### B. Pharmacokinetic Profiles of DNG-EE Extended-Release Formulations

### Example 3: Pharmacokinetic Profile of DNG-EE Extended-Release Formulations (Once Daily Doses for 7 Days)

The information described herein is an extract of the results of a Phase 1 clinical trial, namely an open-label, randomized, cross-over, balanced block study to evaluate the relative bioavailability from four dienogest (DNG) and ethinylestradiol (EE) containing modified and immediate release formulations after repeated oral administration in healthy premenopausal women.

### Materials and Methods

### Objectives

The main objective of the present trial was to assess the relative bioavailability of oral test preparations containing 1 or 2 mg dienogest and 10 or 20 μg ethinylestradiol (Test IMP: dienogest/ethinylestradiol modified release tablets, manufactured by León Farma S.A., Spain) as compared to a market standard (Reference IMP: Valette™ immediate release tablet, containing dienogest 2 mg/ethinylestradiol 30 μg; company responsible for placing the product on the market: Jenapharm GmbH, Germany) after oral administration of a multiple dose once daily for 7 days under fasting conditions in four different periods, at least 7 days apart.

In order to investigate the relative bioavailability of the products, the pre-defined confidence intervals were calculated for the ratios (test/reference) of the primary endpoints AUC(0-τ), Cmax,ss, and Tmax,ss of dienogest and ethinylestradiol. These confidence intervals were compared with the corresponding acceptance ranges. These endpoints underwent descriptive and comparative statistical evaluation.

The secondary objective of the present trial was to investigate the safety of the preparations on the basis of safety clinical and laboratory examinations (at the beginning and at the end of the trial) and registration of adverse events and/or adverse drug reactions.

### Methodology

The study was conducted as a single centre, open-label, multiple dose, crossover, randomized, four-treatment, four-period study in healthy premenopausal female volunteers of between 18 and 40 years of age and BMI within the range (including the borders) of 18.5 to 30.0 kg/m², with duration of hospitalization of approximately 26 hours (day 6 to 7) and with a real wash-out period of 7 days after the last (7th) dose in each period in all subjects.

### Subjects

enrolled (study population): 25
screened only: 6
randomized (safety analysis population): 19
completed: 14
data set for statistical analysis (including the available samples of the drop-outs and replacement of drop-outs): 19
data set for statistical analysis: 14

### Test Products (Product, Dose and Mode of Administration)

TEST 1: Dienogest (DNG) (2 mg)/Ethinylestradiol (EE) (20 μg) modified release tablet; oral/1 modified release tablet once daily for 7 days.
TEST 2: Dienogest (DNG) (1 mg)/Ethinylestradiol (EE) (10 μg) modified release tablet; oral/1 modified release tablet once daily for 7 days.
TEST 3: Dienogest (DNG) (2 mg)/Ethinylestradiol (EE) (10 μg) modified release tablet; oral/1 modified release tablet once daily for 7 days.

The qualitative and quantitative formulation of the tested modified release formulations, as well as the method of producing thereof is as detailed in Example 1 (Lactose—HPMC: Formulations 6, 7 and 8).

Reference product (product, dose and mode of administration) Valette™ immediate release tablets (VALETTE™; Jenapharm GmbH & Co. KG, Germany): 2 mg dienogest (DNG) and 30 μg ethinylestradiol (EE) per immediate release tablet; oral/1 immediate release tablet once daily for 7 days.

### Duration of Treatment

The volunteers swallowed under fasting conditions in each study period an oral daily dose of 1 modified release tablet either Test 1 or Test 2 or Test 3 IMP or 1 immediate release tablet of the Reference IMP in accordance with the randomization schedule.

### Results

Fourteen volunteers completed the trial according to the protocol. The statistical BA evaluation was based on the data of 14 study completers (per protocol set).

### Pharmacokinetics

The primary and secondary endpoints of the statistical analysis of DNG and EE after an oral multiple dose of 1 modified release tablet (once daily for 7 days) of the Test 1, Test 2, Test 3 IMPs or 1 immediate release tablet (once daily for 7 days) of Reference drug of the 14 subjects who were subject to statistical evaluation are summarized in Tables 14 and 15.

TABLE 14
Pharmacokinetic profiles of DNG for DNG-EE extended-
release formulations (Once Daily Doses for 7 Days)
DIENOGEST

| | | | | | | | |
|---|---|---|---|---|---|---|---|
| | geom. | arithm. | | | | | |
| Variable [unit] | mean | mean | SD | CV | range | median | N |

TEST 1 (2 mg dienogest and 20 μg ethinylestradiol per modified release tablet)

| | | | | | | | |
|---|---|---|---|---|---|---|---|
| AUC(0-τ) [ng*h/mL] | 716.7 | 731.6 | 158.6 | 21.7 | 542.1-1085.2 | 714.8 | 14 |
| Cmax, ss [ng/mL] | 58.2 | 59.3 | 12.3 | 20.7 | 46.6-91.4 | 55.8 | 14 |
| Tmax, ss [h] | — | 3.750 | 0.700 | 18.7 | 3.000-5.000 | 4.000 | 14 |

TEST 2 (1 mg dienogest and 10 μg ethinylestradiol per modified release tablet)

| | | | | | | | |
|---|---|---|---|---|---|---|---|
| AUC(0-τ) [ng*h/mL] | 358.1 | 366.5 | 80.3 | 21.9 | 222.1-524.8 | 372.4 | 14 |
| Cmax, ss [ng/mL] | 28.1 | 28.6 | 5.6 | 19.7 | 20.0-40.9 | 26.8 | 14 |
| Tmax, ss [h] | — | 3.857 | 0.745 | 19.3 | 3.000-5.000 | 4.000 | 14 |

TEST 3 (2 mg dienogest and 10 μg ethinylestradiol per modified release tablet)

| | | | | | | | |
|---|---|---|---|---|---|---|---|
| AUC(0-τ) [ng*h/mL] | 721.1 | 735.0 | 154.3 | 21.0 | 569.2-1006.5 | 719.1 | 14 |
| Cmax, ss [ng/mL] | 55.4 | 56.2 | 10.1 | 17.9 | 41.7-76.4 | 56.0 | 14 |
| Tmax [h] | — | 3.754 | 0.752 | 20.0 | 2.500-5.000 | 4.000 | 14 |

REFERENCE (2 mg dienogest and 30 μg ethinylestradiol per immediate release tablet)

| | | | | | | | |
|---|---|---|---|---|---|---|---|
| AUC(0-τ) [ng*h/mL] | 707.2 | 720.3 | 140.9 | 19.6 | 466.8-1000.2 | 731.1 | 14 |
| Cmax, ss [ng/mL] | 74.6 | 75.4 | 12.5 | 16.5 | 60.8-109.0 | 74.0 | 14 |
| Tmax, ss [h] | — | 1.321 | 0.805 | 60.9 | 0.333-3.000 | 1.000 | 14 |

TABLE 15
Pharmacokinetic profiles of EE for DNG-EE extended-
release formulations (Once Daily Doses for 7 Days)
ETHINYLESTRADIOL

| | | | | | | | |
|---|---|---|---|---|---|---|---|
| | geom. | arithm. | | | | | |
| Variable [unit] | mean | mean | SD | CV | range | median | N |

TEST 1 (2 mg dienogest and 20 μg ethinylestradiol per modified release tablet)

| | | | | | | | |
|---|---|---|---|---|---|---|---|
| AUC(0-τ) [pg*h/mL] | 683.3 | 706.3 | 196.4 | 27.8 | 465.4-1221.1 | 691.4 | 14 |
| Cmax, ss [pg/mL] | 59.9 | 63.6 | 23.3 | 36.6 | 33.3-112.0 | 60.0 | 14 |
| Tmax, ss [h] | — | 3.754 | 1.242 | 33.1 | 2.000-6.033 | 4.000 | 14 |

TEST 2 (1 mg dienogest and 10 μg ethinylestradiol per modified release tablet)

| | | | | | | | |
|---|---|---|---|---|---|---|---|
| AUC(0-τ) [pg*h/mL] | 342.4 | 351.9 | 90.0 | 25.6 | 239.6-587.9 | 331.9 | 14 |
| Cmax, ss [pg/mL] | 31.2 | 32.3 | 9.5 | 29.5 | 23.1-56.4 | 29.9 | 14 |
| Tmax, ss [h] | — | 3.571 | 0.805 | 22.5 | 2.500-5.000 | 3.500 | 14 |

TEST 3 (2 mg dienogest and 10 μg ethinylestradiol per modified release tablet)

| | | | | | | | |
|---|---|---|---|---|---|---|---|
| AUC(0-τ) [pg*h/mL] | 326.2 | 338.4 | 99.0 | 29.3 | 188.0-590.7 | 330.0 | 14 |
| Cmax, ss [pg/mL] | 30.4 | 33.2 | 15.8 | 47.5 | 15.9-75.5 | 28.4 | 14 |
| Tmax, ss [h] | — | 3.468 | 0.746 | 21.5 | 2.000-5.000 | 3.500 | 14 |

REFERENCE (2 mg dienogest and 30 μg ethinylestradiol per immediate release tablet)

| | | | | | | | |
|---|---|---|---|---|---|---|---|
| AUC(0-τ) [pg*h/mL] | 1030.5 | 1072.8 | 345.2 | 32.2 | 636.8-2055.1 | 974.0 | 14 |
| Cmax, ss [pg/mL] | 129.0 | 135.2 | 44.9 | 33.2 | 78.2-250.0 | 132.5 | 14 |
| Tmax, ss [h] | — | 1.345 | 0.504 | 37.5 | 0.667-2.000 | 1.250 | 14 |

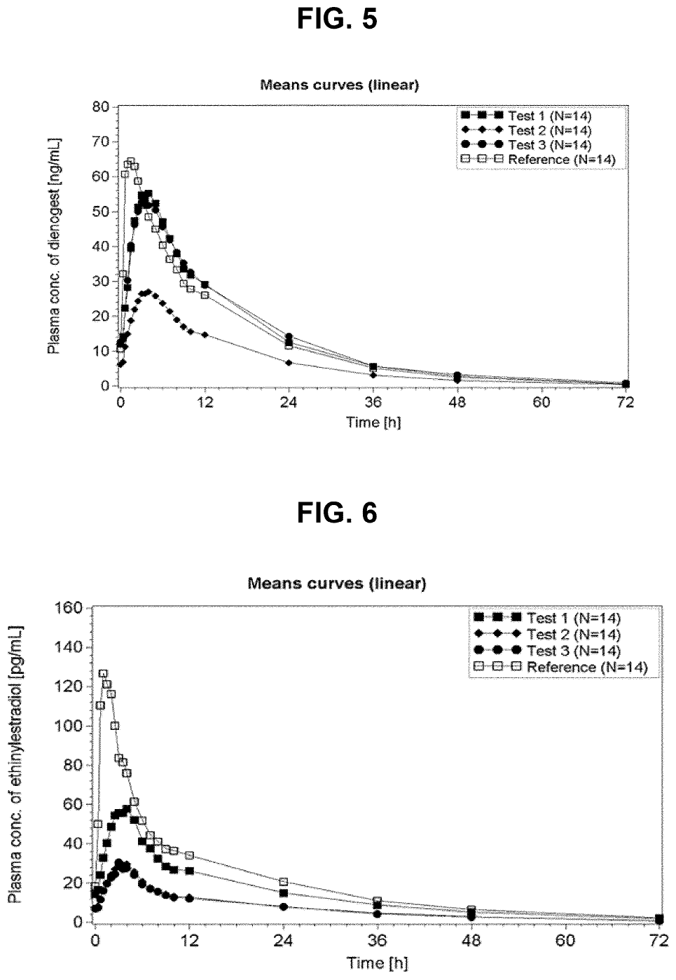

The concentration-time curve of DNG after administration of an oral multiple dose of 1 modified release tablet of the Test 1, Test 2, Test 3 IMPs and 1 immediate release tablet of Reference drug once daily for 7 days are to be found in FIG. 5 for the all preparations (arithmetic means).

The concentration-time curve of EE after administration of an oral multiple dose of 1 modified release tablet of the Test 1, Test 2, Test 3 IMPs and 1 immediate release tablet of the Reference drug are to be found in FIG. 6 for all preparations (arithmetic means).

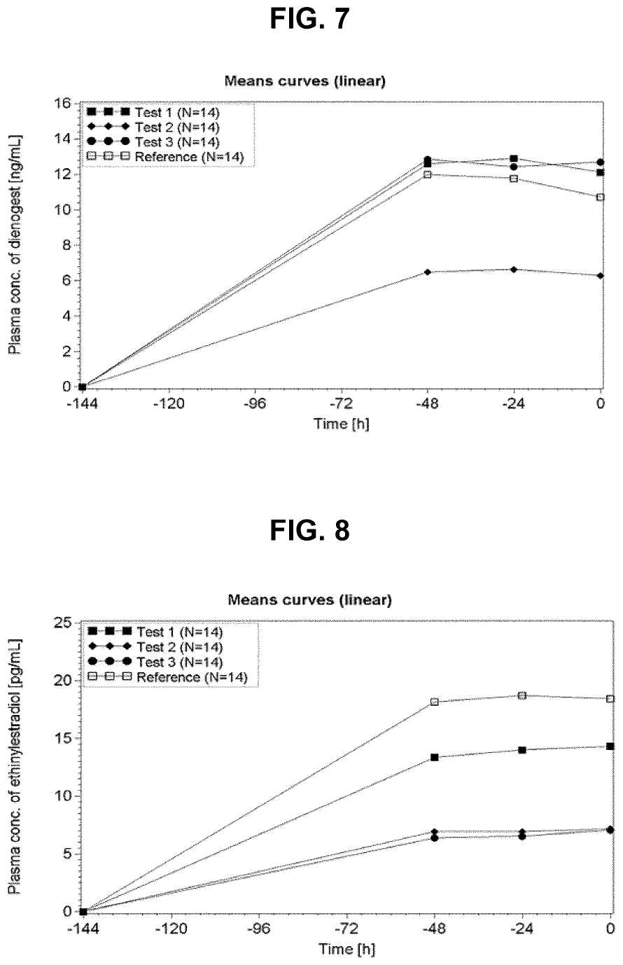

The achieving of steady-state for both DNG and EE concentrations before the 7th dose is presented in text FIGS. 7 and 8.

### Safety:

The 3 Test formulations and the Reference drug were equally well tolerated. The pattern of almost all AEs is in the line with what has to be expected after administration of dienogest and ethinylestradiol (mentioned in the SmPC of the reference IMP Valette™)

### Overall Study Conclusions

In this study, all tested formulations containing dienogest and ethinylestradiol were safe and well tolerated after multiple dose of once daily administration for 7 days in healthy premenopausal females. The tolerability of the three investigational prolonged release formulations was comparable to the reference product.

Attainment of steady-state: pre-dose blood samples were taken on days 1, 5, 6, and 7 of dosing. For all products and for both analytes, the pre-dose levels did not increase after day 5.

After repeated daily intake over 7 days of administration, the main pharmacokinetic characteristics of the three Test PR formulations—Dienogest/ethinylestradiol modified release tablets were:
Tmax was shifted from 1.5 hours to approximately 4 hours in the PR formulations.
AUC0-24 h was strictly proportional compared to the immediate release reference product.
Peak Through Fluctuations were reduced in the PR formulations compared to immediate release reference product (Cmax was reduced by approx. 25% and Cmin was slightly higher in the PR formulations).

### Example 4: Pharmacokinetic Profile of DNG-EE Extended-Release Formulations (Single-Dose: Fasting and Fed Conditions)

An open-label, single dose, randomized, two-period crossover, Phase I pharmacokinetic study with modified release Dienogest/Ethinyl Estradiol 2.00 mg/0.02 mg film-coated tablets (Test) administered with or without food in healthy post-menopausal female volunteers. The qualitative and quantitative formulation of the tested modified release formulation correspond to Example 1 (Lactose—HPMC: Formulation 8).

The main objective was to assess the pharmacokinetics of the oral test preparation after single dose administration under fasting and fed conditions in two different periods, at least 7 days apart.

The clinical trial was conducted as a single center, open-label, two-treatment, two-period, crossover study in healthy post-menopausal female volunteers, with a duration of hospitalization of about 27 hours (up to 15 hours before and until 12 hours after the dosing on day 1) and with an actual wash-out period of 7 days between both treatment periods for all subjects. Each volunteer received in a crossover fashion randomly an oral single dose of 1 film-coated tablet (=Dienogest 2 mg and Ethinyl Estradiol 0.02 mg) of the TEST IMP either under fasting or under fed conditions (i.e. 30 minutes after intake of a standard high-fat breakfast) on two single occasions.

### Pharmacokinetics:

Primary endpoints: AUC(0-72 h) and Cₘₐₓ of dienogest and ethinyl estradiol
Secondary endpoints: tmax of dienogest and ethinyl estradiol
Safety: Safety clinical and laboratory examinations (at the beginning and at the end of the trial) and registration of adverse events and/or adverse drug reactions.

### Results

A total number of 18 volunteers were randomized and treated with study medication. Eighteen volunteers completed the trial according to the protocol. The samples of 18 study completers were analysed. The comparative statistical evaluation was based on the data of 18 subjects (per protocol set).

The plasma exposure of dienogest (DNG) and ethinylestradiol (EE) following single-dose administration in the fasted and fed state are shown in Tables 16 and 17. There was no food effect observed for the prolonged release formulation (2 mg DNG and 20 μg EE) in the single dose study following intake of a standard high-fat breakfast 30 minutes before administration compared to fasted state.

TABLE 16
Pharmacokinetic profiles of DNG for DNG-EE extended-release
Formulation 8 following Single-Dose in Fasted and Fed states

| | | Geom. | Arithm. | | | | | |
|---|---|---|---|---|---|---|---|---|
| Test | Variable | mean | mean | SD | CV | Range | Median | N |
| ‘A’ under | AUC(_{0-72 h)} | 596.98 | 604.64 | 103.60 | 17.1 | 483.24-859.36 | 600.16 | 18 |
| fasting | [ng*h/mL] | | | | | | | |
| conditions | Cₘₐₓ [ng/mL] | 38.04 | 38.78 | 7.90 | 20.4 | 26.77-54.69 | 38.45 | 18 |
| | tₘₐₓ [h] | — | 4.038 | 0.695 | 17.2 | 3.000-6.000 | 4.017 | 18 |
| ‘B’ under fed | AUC(_{0-72 h)} | 616.17 | 626.36 | 120.75 | 19.3 | 436.97-938.03 | 594.21 | 18 |
| conditions | [ng*h/mL] | | | | | | | |
| | Cₘₐₓ [ng/mL] | 43.44 | 44.08 | 7.70 | 17.5 | 30.21-58.36 | 44.76 | 18 |
| | tₘₐₓ [h] | — | 3.254 | 0.814 | 25.0 | 1.500-4.533 | 3.000 | 18 |

Abbreviations:
AUC(_{0-72 h)} = Area under the concentration-time curve from 0-72 h;
Cₘₐₓ = Maximum observed plasma concentration;
tₘₐₓ = Time until Cₘₐₓ is reached;
CV = Coefficient of variance;
DNG = Dienogest;
EE—Ethinyl estradiol;
h—Hour;
N = Number;
SD = Standard deviation

TABLE 17
Pharmacokinetic profiles of EE for DNG-EE extended-release Formulation
8 following Single-Dose in Fasted and Fed states

| | | Geom. | Arithm. | | | | | |
|---|---|---|---|---|---|---|---|---|
| Test | Variable | mean | mean | SD | CV | Range | Median | N |
| ‘A’ under | AUC_{0-72 h)} | 454.85 | 483.11 | 169.34 | 35.1 | 241.40-788.22 | 479.39 | 18 |
| fasting | [pg*h/mL] | | | | | | | |
| conditions | Cₘₐₓ [pg/mL] | 30.32 | 32.42 | 12.11 | 37.4 | 11.69-57.77 | 29.33 | 18 |
| | tₘₐₓ [h] | — | 4.255 | 0.550 | 12.9 | 3.000-5.017 | 4.500 | 18 |
| ‘B’ under fed | AUC_{0-72 h)} | 484.82 | 510.45 | 162.84 | 31.9 | 269.29-781.86 | 514.16 | 18 |
| conditions | [pg*h/mL] | | | | | | | |
| | Cₘₐₓ [pg/mL] | 32.02 | 33.50 | 10.07 | 30.1 | 18.43-51.11 | 33.82 | 18 |
| | tₘₐₓ [h] | — | 3.754 | 0.943 | 25.1 | 2.000-5.000 | 4.250 | 18 |

Abbreviations:
AUC(_{0-72 h)} = Area under the concentration-time curve from 0-72 h;
Cₘₐₓ = Maximum observed plasma concentration;
tₘₐₓ = Time until Cₘₐₓ is reached;
CV = Coefficient of variance;
DNG = Dienogest;
EE—Ethinyl estradiol;
h—Hour;
N = Number;
SD = Standard deviation

### C. Efficacy of DNG-EE Extended-Release Formulations

### Example 5: Contraceptive Efficacy of DNG-EE Extended-Release Formulations Versus DRSP-EE Formulations (Hoogland Score, Langren Score)

Described below is an extract of the results of a single center, phase II, open label randomized clinical trial to evaluate the inhibition of ovulation of three prolonged release formulations containing a combination of dienogest (DNG) and ethinyl estradiol (EE) versus a flexible regimen contraceptive containing drospirenone (DRSP) 3 mg and ethinyl estradiol (EE) 20 μg in 100 healthy women.

### Materials and Methods

### Objectives

The primary objective was the inhibition of ovarian activity (Hoogland score) of oral modified release preparations containing 1 or 2 mg DNG and 10 or 20 μg EE compared with the immediate release oral contraceptive Velmari® (EE 0.02 mg/DRSP 3 mg) in Treatment Cycle (TC) 1 and TC4.

The bleeding pattern; serum levels of progesterone, estradiol (EE), follicle-stimulating hormone (FSH), and luteinizing hormone (LH); safety and tolerability; and the Landgren score if an ovulation had been observed sonographically in TC1 or TC4 were secondary objectives of the study.

### Methodology

Healthy women were randomized in a 1:1:1:1 ratio to receive 1 of 4 unblinded treatments: ethinyl estradiol (EE)/dienogest (DNG) 10 μg/1 mg, or EE/DNG 10 μg/2 mg, or EE/DNG 20 μg/2 mg, or Velmari® Langzyklus 0.02/3 mg tablets (EE 20 μg/DRSP 3 mg).

### Number of Subjects (Planned and Analyzed)

Planned: 100; Actual: 100; Completed: 84; Analyzed: 100.

### Determination of Sample Size

There was no formal sample size calculation. The sample size was set to 25 subjects per group (total 100 subjects allocated in the trial) and was based on methodology of DNG-mediated inhibition of ovulation as described in the literature (Klipping et al 2012).

### Diagnosis and Main Criteria for Inclusion

The study sample were healthy women (18-35 years, inclusive) with a history of regular cycles and no evident gynecological abnormalities. Women with conditions or characteristics that might have had an impact on the aims of the study or meant a safety risk for the subject were excluded. In particular, pregnancy, a likelihood of residual pharmaceutical hormones in the body, or a history of thrombophlebitis, venous or arterial thromboembolic diseases led to exclusion.

### Test and Reference Products (Product, Dose and Mode of Administration)

T1: EE/DNG 10 μg/1 mg
T2: EE/DNG 10 μg/2 mg
T3: EE/DNG 20 μg/2 mg
T4: Velmari® Langzyklus 0.02/3 mg tablets (EE 20 μg/DRSP 3 mg)

### One Tablet/Day Oral Administration

Subjects received an investigational medicinal product (IMP) kit containing 4 blisters of 24 tablets to cover the 87-day treatment period. On Day 91, subjects taking test products were given 1 blister of 28 tablets and subjects taking Velmari® were given 2 blisters each containing 24 tablets.

Both test and reference products were manufactured and packaged by Laboratorios León Farma, S.A. (León, Spain).

The qualitative and quantitative formulation of the tested modified release formulations, as well as the method of producing thereof, is as detailed in Example 1 (Lactose—HPMC) using the proportions of T1-T3 shown above.

### Duration of Treatment

Treatment phase (TC1 to TC4): 119 days. TC1 to TC3 corresponds to a first treatment period (extended) 87 days intake+4 hormone-free days (91); and TC4 to a second treatment period of 24 days intake+4 hormone-free days. TC1 corresponds to days 1 to 27.

### Statistical Methods

This was an exploratory study. All data were summarized with sample statistics or frequency tables as appropriate.

### Results

One hundred subjects were randomized and received at least 1 dose of IMP, and 84 subjects completed the study.

### Efficacy Results

### Hoogland Score:

Inhibition of ovulation was measured using the Hoogland score (Hoogland and Skouby 1993), which reflects the ovarian status, during TC1 and TC4, based on the observations at the scheduled visits.

The Hoogland score combines follicle size in mm and progesterone/estradiol serum concentrations in nmol/L. The scoring system is detailed in Table 18.

TABLE 18
Hoogland Score Evaluation

| | Size of Largest | | | | | | | | | |
|---|---|---|---|---|---|---|---|---|---|---|
| Score | Follicle | | Progesterone | | | | Estradiol | | | |
| 1: No activity | ≤10 | mm | — | | — | | — | | — | |
| 2: Potential | >10 | mm | — | | — | | — | | — | |
| activity | | | | | | | | | | |
| 3: Nonactive | >13 | mm | — | | — | | ≤27.2 | pg/mL | ≤0.1 | nmol/L |
| FLS | | | | | | | | | | |
| 4: Active FLS | >13 | mm | ≤1.6 | ng/mL | ≤5 | nmol/L | >27.2 | pg/mL | >0.1 | nmol/L |
| 5: LUF | >13 | mm persisting | >1.6 | ng/mL | >5 | nmol/L | >27.2 | pg/mL | >0.1 | nmol/L |
| 6: Ovulation | >13 | mm ruptured | >1.6 | ng/mL | >5 | nmol/L | >27.2 | pg/mL | >0.1 | nmol/L |

FLS = follicle-like structure;
LUF = luteinized unruptured follicle

The maximum Hoogland score observed during the study was used for the efficacy assessment. Three categories were defined based on the Hoogland score:
1 or 2: no or minimum ovarian activity
3 or 4: residual ovarian activity
5 or 6: high ovarian activity including ovulation

A Hoogland score 1 to 4 was defined as “inhibition of ovulation” for the efficacy assessment. Based on this definition, the inhibition rate in the treatment groups T3 and Velmari® was 100% in TC1 and TC4. For T2, the inhibition rates were 100% and 95.5% in TC1 and TC4; for T1, the inhibition rates were 96.0% and 90.9% in TC1 and TC4 (Table 19).

TABLE 19
Inhibition Rates

| | | T1 | | T2 | | T3 | Velmari ® |
|---|---|---|---|---|---|---|---|
| TC | | N = 25 n | | N = 24 n | | N = 25 n | N = 24 |
| No. | Inhibition | (%) | | (%) | | (%) | n (%) |
| TC 1 | No | 1 | (4.0) | 0 | | 0 | 0 |
| | Yes | 24 | (96.0) | 24 | (100.0) | 25 (100.0) | 24 (100.0) |
| TC 4 | No | 2 | (9.1) | 1 | (4.5) | 0 | 0 |
| | Yes | 20 | (90.9) | 21 | (95.5) | 22 (100.0) | 20 (100.0) |

### Landgren Score:

The Landgren score was determined in TC1 and TC4 only if an ovulation was suspected in the TVU examination and if the corresponding Hoogland score was 5 or 6.

According to Landgren (Landgren et al 1980), a normal ovulation can be verified by a progesterone level of >16 nmol/L on a minimum of 5 consecutive days. For the purpose of this study, the Landgren score was positive if in 3 progesterone measurements within 5 days the progesterone was >16 nmol/L.

The Landgren score was determined 5 times (T1: 4 cases; T2: 1 case). It was positive in one T1 subject during TC4 and negative in all other cases.

### Example 6: Contraceptive Efficacy of DNG-EE Extended-Release Formulations Versus DRSP-EE Formulations (Pearl Index) (9 Cycles)

A multicentre, double-blind, double-dummy, randomised trial on the contraceptive efficacy, tolerability and safety of Formulation 17 (Example 2, dienogest (DNG) 2 mg/ethinyl estradiol (EE) 0.02 mg) during 9 cycles in comparison with drospirenone (DRSP) 3 mg/ethinyl estradiol (EE) 0.02 mg was conducted.

A total of 782 (76.1%) subjects completed the trial (559 [67.2%] subjects in the DNG/EE group (Formulation 17) and 223 [75.9%] subjects in the DRSP/EE group. The mean (SD) subjects' age in the total population was 27.4 (6.87) years ranging from 18 to 45 years. The majority of women were 35 years of age or younger.

### 24-Hour Miss-Pill Window

If the subject was less than 24 hours late from her usual time in taking tablet, the missed tablet was to be taken as soon as it was remembered, and the next tablet was to be taken at the usual time. If she was more than 24 hours late from her usual time in taking an active tablet, she was to take the tablet as soon as she remembered, even if that meant taking up to two tablets at the same time (i.e., the late tablet with the current tablet). Then the next day's tablet was to be taken at the usual time. Taking 2 tablets in one day was considered acceptable and did not lead to overdosing. In the case of more than 2 missed tablets within a cycle, the investigator was to advise the subject to re-enter the medication cycle correctly, so that the subject re-entered treatment as though she had been taking pills consecutively since Day 1 of the respective cycle. Missed tablets were to remain in the blister pack.

### Efficacy Results:

The primary endpoint was defined as the overall Pearl Index (PI) from exposure cycles in women aged ≤35 years (at the time of trial enrolment) and was based on confirmed on-drug pregnancies. The Full Analysis Set (FAS) was the primary set for efficacy analysis. As shown in Table 20, the overall PI (95% CI) in the DNG/EE group (Formulation 7) based on 605 subjects, 2 confirmed on-drug pregnancies and 4650 exposure cycles was 0.6 (0.07, 2.02) compared to an overall PI (95% CI) in the DRSP/EE group of 0 (NC, 2.55) based on 246 subjects, no confirmed on-drug pregnancies and 1883 exposure cycles.

TABLE 20
Pearl Index Based on Exposure Cycles and Confirmed On-Drug
Pregnancies in Women Aged ≤35 Years (Full Analysis Set)

| | DNG/EE | | DRSP/EE | |
|---|---|---|---|---|
| | (N - 605) | | (N = 246) | |
| Total number of exposure cycles | 4650 | | 1883 | |
| Confirmed on-drug pregnancy | | | | |
| Subjects with pregnancy (n [%]) | 2 | (0.3) | 0 | |
| Subjects without pregnancy (n | 603 | (99.7) | 246 | (100.0) |
| [%]) | | | | |
| Pearl Index (95% CI) | 0.6 | (0.07, 2.02) | 0 | (NC, 2.55) |

N: Number of subjects aged ≤35 years in Full Analysis Set; n: Number of subjects with data available; %: Percentage based on N; CI: Confidence interval, NC: not calculable.

Based on the data in Table 20, Formulation 17 (Example 2, dienogest (DNG) 2 mg/ethinyl estradiol (EE) 0.02 mg) has a miss pill window of 24 hours (efficacy of the product is maintained).

By contrast, Valette™ (combined immediate release oral contraceptive with 0.03 mg ethinyl estradiol and 2 mg dienogest) in a 21/7 regimen has a miss pill window of only 12 hours. The 24-hour miss pill window of Formulation 17, together with the 24/4 daily intake, greatly improves a patient's adherence to the product and reduces contraceptive failure.

### Example 7: Contraceptive Efficacy of DNG-EE Extended-Release Formulations (Pearl Index) (13 Cycles)

A multicentre, uncontrolled trial on the contraceptive efficacy, safety, tolerability and pharmacokinetics of Formulation 17 (Example 2, dienogest (DNG) 2 mg/ethinyl estradiol (EE) 0.02 mg) during 13 cycles was conducted.

A total of 609 (70.8%) subjects completed the trial, and 251 (29.2%) subjects prematurely terminated the trial. The most common primary reasons for discontinuation were AEs (113 subjects, 13.1%) and at subject's own request (withdrawal of consent) (73 subjects, 8.5%). At screening, the mean (SD) subjects' age was 27.8 (7.06) years ranging from 15 to 45 years.

### 24-Hour Miss-Pill Window

If the subject was less than 24 hours late from her usual time in taking tablet, the missed tablet was to be taken as soon as it was remembered, and the next tablet was to be taken at the usual time. If she was more than 24 hours late from her usual time in taking an active tablet, she was to take the tablet as soon as she remembered, even if that meant taking up to two tablets at the same time (i.e., the late tablet with the current tablet). Then the next day's tablet was to be taken at the usual time. Taking 2 tablets in one day was considered acceptable and did not lead to overdosing. In the case of more than 2 missed tablets within a cycle, the investigator was to advise the subject to re-enter the medication cycle correctly, so that the subject re-entered treatment as though she had been taking pills consecutively since Day 1 of the respective cycle. Missed tablets were to remain in the blister pack.

### Efficacy Results:

The primary endpoint was defined as the overall Pearl Index (PI) from exposure cycles in women aged ≤35 years (at the time of trial enrolment) and was based on confirmed on-drug pregnancies. The Full Analysis Set (FAS) was the primary set for the efficacy analysis. A total of 704 subjects aged ≤35 years with 7476 exposure cycles were analysed. During these cycles no subject became pregnant, leading to a PI (95% CI) of 0 (NC; 0.64) as summarized in Table 21.

TABLE 21
Pearl Index Based on Exposure Cycles and Confirmed On-Drug
Pregnancies in Women Aged ≤35 Years (Full Analysis Set)

| | DNG/EE | |
|---|---|---|
| | (N - 704) | |
| Total number of exposure cycles | 7476 | |
| Confirmed on-drug pregnancy | | |
| Subjects with pregnancy (n [%]) | 0 | |
| Subjects without pregnancy (n [%]) | 704 | (100.0) |
| Pearl Index (95% CI) | 0 | (NC, 0.64) |

Following the CHMP Guideline on Clinical Investigation of Steroid Contraceptives in Women (EMEA/CPMP/EWP/519/98 Rev 1), the number of cycles collected should be at least large enough to give the overall Pearl Index (PI) with a 95% confidence interval-such that the difference between the upper limit of the confidence interval and the point estimate does not exceed 1. The DNG-EE data from the two studies in Examples 6 and 7 were pooled in order to calculation the overall P1. In total, the analysed number of exposure cycles in both trials in women aged ≤35 years was 12 126 and in all women 14,597.

A total of 2 confirmed on-drug pregnancies including one method failure pregnancy and one user failure pregnancy, were observed in women who used Formulation 17 (Example 2, dienogest (DNG) 2 mg P ethinyl estradiol (EE) 0.02 mg) up to 13×28-day. Both pregnancies were reported for subjects ≤35 years. As summarized in Table 22, an overview of number of cycles and overall PIs, PIs based on evaluable cycles and method failure PIs (confirmed pregnancies) is presented for all women and for women aged ≤35 years.

TABLE 22
Pearl Indices DNG-EE extended-release formulations_{—}
Based on Exposure Cycles, Evaluable Cycles, Perfect Cycles
and Confirmed On-Drug Pregnancies in Pooled data of Examples
6 and 7 in All Women versus Women Aged ≤35 Years

| | Formulation 17 | |
|---|---|---|
| | (DNG/EE 2 mg/0.02 mg) | |
| | Women Aged ≤35 | All Woman |
| | Years (N = 1309) | (N = 1576) |
| Overall Pearl Index | | |
| Total number of exposure | 12126 | 14597 |
| cycles | | |
| Confirmed on-drug pregnancy | 2 (0.2) | 2 (0.1) |
| (n [%]) | | |
| Pearl Index (95% CI) | 0.2 (0.03, 0.77) | 0.2 (0.02, 0.64) |
| Pearl Index for evaluable | | |
| cycles | | |
| Total number of exposure | 9624 | 11808 |
| cycles | | |
| Confirmed on-drug pregnancy | 2 (0.2) | 2 (0.1) |
| (n [%]) | | |
| Pearl Index (95% CI) | 0.3 (0.03, 0.98) | 0.2 (0.03, 0.8) |
| Pearl Index for method | | |
| failures | | |
| Total number of exposure | 6415 | 8006 |
| cycles | | |
| Confirmed on-drug pregnancy | 2 (0.2) | 2 (0.1) |
| (n [%]) | | |
| Pearl Index (95% CI) | 0.4 (0.05, 1.46) | 0.3 (0.04, 1.17) |

Considering the two confirmed on-drug pregnancies reported in the study for women aged ≤35 years, the PIs (95% CIs) calculated for pooled data were 0.2 (0.03, 0.77) for 12,126 exposure cycles (primary endpoint), 0.3 (0.03, 0.98) for 9,624 evaluable cycles and 0.4 (0.05, 1.46) for 6,415 perfect cycles. In the whole population of women, the PIs (95% CI) calculated for pooled data were 0.2 (0.02, 0.64) for 14,597 exposure cycles, 0.2 (0.03, 0.8) for 11,808 evaluable cycles and 0.3 (0.04, 1.17) for 8,006 perfect cycles.

Based on the data in Tables 21 and 22, Formulation 17 (Example 2, dienogest (DNG) 2 mg/ethinyl estradiol (EE) 0.02 mg) has a miss pill window of 24 hours (efficacy of the product is maintained). By contrast, Valette™ (combined immediate release oral contraceptive with 0.03 mg ethinyl estradiol and 2 mg dienogest) in a 21/7 regimen has a miss pill window of only 12 hours. The 24-hour miss pill window of Formulation 17, together with the 24/4 daily intake, greatly improves a patient's adherence to the product and reduces contraceptive failure.

### D. Tolerability and Safety of DNG-EE Extended-Release Formulations

### Example 8: Tolerability and Safety of DNG-EE Extended-Release Formulations Versus DRSP-EE Formulations (Bleeding Patterns and Adverse Events)

Described below is an extract of the results of a single center, phase II, open label randomized clinical trial to evaluate the inhibition of ovulation of three prolonged release formulations containing a combination of dienogest (DNG) and ethinyl estradiol (EE) versus a flexible regimen contraceptive containing drospirenone (DRSP) 3 mg and ethinyl estradiol (EE) 20 μg in 100 healthy women.

### Materials and Methods

### Objectives

The objective was to study the tolerability (in terms of bleeding pattern) of oral modified release preparations containing 1 or 2 mg DNG and 10 or 20 μg EE compared with the immediate release oral contraceptive Velmari® (EE 0.02 mg/DRSP 3 mg) in Treatment Cycle (TC) 1 and TC4.

### Methodology

Healthy women were randomized in a 1:1:1:1 ratio to receive 1 of 4 unblinded treatments: ethinyl estradiol (EE)/dienogest (DNG) 10 μg/1 mg, or EE/DNG 10 μg/2 mg, or EE/DNG 20 μg/2 mg, or Velmari® Langzyklus 0.02/3 mg tablets (EE 20 μg/DRSP 3 mg).
Number of Subjects (Planned and Analyzed)
Planned: 100; Actual: 100; Completed: 84; Analyzed: 100.

### Determination of Sample Size

There was no formal sample size calculation. The sample size was set to 25 subjects per group (total 100 subjects allocated in the trial) and was based on methodology of DNG-mediated inhibition of ovulation as described in the literature (Klipping et al 2012).

### Diagnosis and Main Criteria for Inclusion

The study sample were healthy women (18-35 years, inclusive) with a history of regular cycles and no evident gynecological abnormalities. Women with conditions or characteristics that might have had an impact on the aims of the study or meant a safety risk for the subject were excluded.

In particular, pregnancy, a likelihood of residual pharmaceutical hormones in the body, or a history of thrombophlebitis, venous or arterial thromboembolic diseases led to exclusion.

### Test and Reference Products (Product, Dose and Mode of Administration)

T1: EE/DNG 10 μg/1 mg
T2: EE/DNG 10 μg/2 mg
T3: EE/DNG 20 μg/2 mg
T4: Velmari® Langzyklus 0.02/3 mg tablets (EE 20 μg/DRSP 3 mg)

### One Tablet/Day Oral Administration

Subjects received an investigational medicinal product (IMP) kit containing 4 blisters of 24 tablets to cover the 87-day treatment period. On Day 91, subjects taking test products were given 1 blister of 28 tablets and subjects taking Velmari® were given 2 blisters each containing 24 tablets.

Both test and reference products were manufactured and packaged by Laboratorios León Farma, S.A. (León, Spain).

The qualitative and quantitative formulation of the tested modified release formulations, as well as the method of producing thereof, is as detailed in Example 1 (Lactose—HPMC) using the proportions of T1-T3 shown above.

### Duration of Treatment

Treatment phase (TC1 to TC4): 119 days. TC1 to TC3 corresponds to a first treatment period (extended) 87 days intake+4 hormone-free days (91); and TC4 to a second treatment period of 24 days intake+4 hormone-free days. TC1 corresponds to days 1 to 27.

### Statistical Methods

This was an exploratory study. All data were summarized with sample statistics or frequency tables as appropriate.

### Results

One hundred subjects were randomized and received at least 1 dose of IMP, and 84 subjects completed the study.

### Bleeding Pattern

The Bleeding Pattern is not an absolute value but relative to other contraceptives and doses. The trial was double-blind and Velmari® was used herein for comparative purposes. Subjects noted the occurrence of vaginal bleeding in a subject diary as none, spotting, light, normal, or heavy bleeding. For the analysis, the following treatment periods were considered and tabulated separately: TC1 (Day 1-24), TC4 (Day 92-119), TC1 to TC3 (Day 1-91), and TC1 to TC4 (Day 1-119). Summary data of TC4 and TC1 to TC3 are shown in Table 23.

“No bleeding” was recorded for the majority of days in all treatment groups, followed by “spotting.” Only a small proportion of days were recorded as light, normal, or heavy bleeding (in descending order) for all treatment groups.

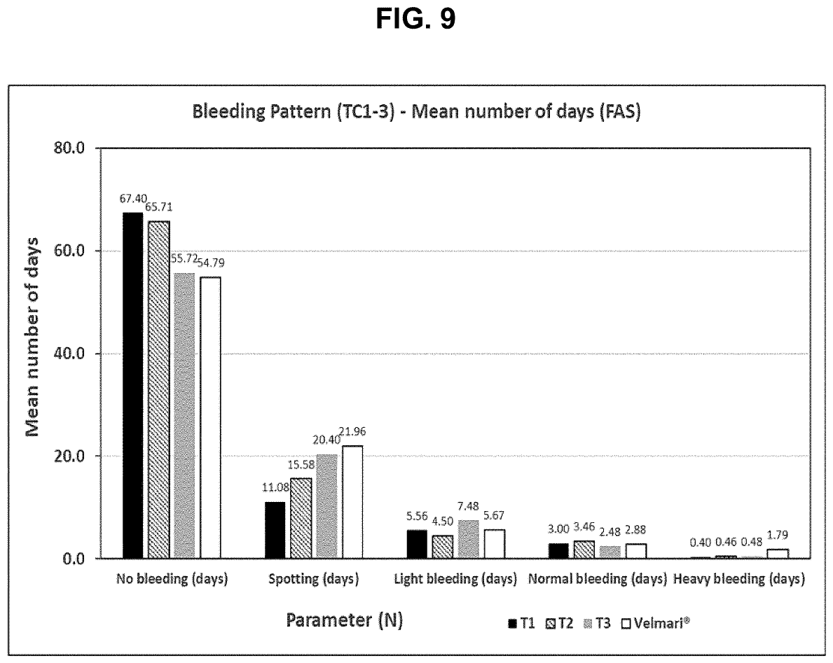

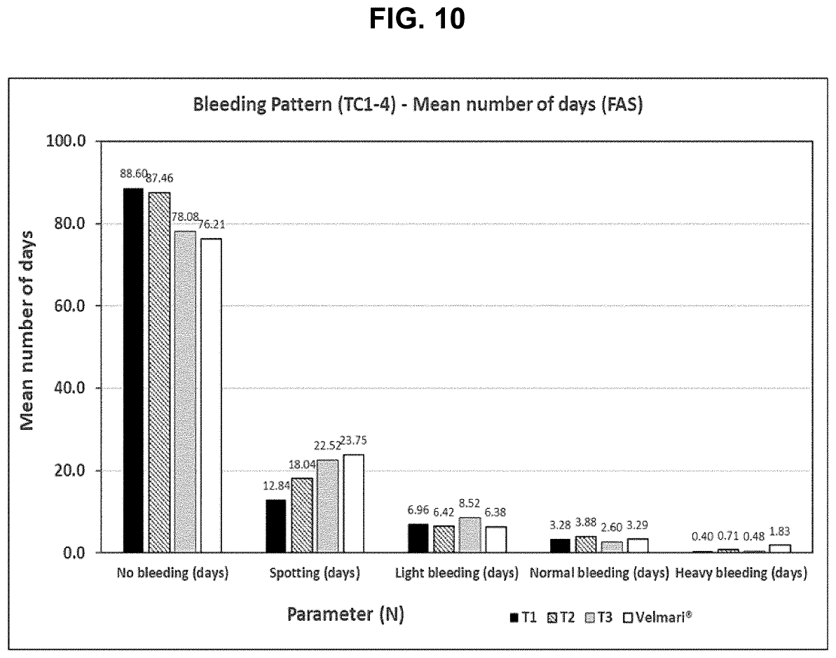

As summarized in Table 23, the bleeding pattern was least favorable in the Velmari® group with a mean value of only 76 bleeding-free days, compared with 78 days for T3, 87 days for T2, and 89 days for T1 (TC1 to TC4 inclusive). In TC4, however, values converged towards 23 to 24 days for all groups. FIG. 9 is a chart summarizing the bleeding patterns in TC1 to TC3; and FIG. 10 is a chart summarizing the bleeding patterns in TC1 to TC4.

TABLE 23
Bleeding Pattern of DNG-EE extended-release formulations versus DRSP-EE formulations

| | Bleeding | | T1 | T2 | T3 | Velmari ® | Total |
|---|---|---|---|---|---|---|---|
| | Category | | N = 25 | N = 25 | N = 25 | N = 25 | N = 100 |
| TC 1 | None | Mean ± SD¹ | 18.7 ± 5.1 | 17.0 ± 5.9 | 13.7 ± 7.5 | 16.7 ± 6.2 | 16.5 ± 6.4 |
| | (days) | Min-Max | 7-24 | 0-24 | 1-25 | 1-24 | 0-25 |
| | Spotting | Mean ± SD | 3.2 ± 3.7 | 4.9 ± 5.5 | 7.9 ± 6.1 | 5.2 ± 5.7 | 5.3 ± 5.5 |
| | (days) | Min-Max | 0-13 | 0-23 | 0-23 | 0-21 | 0-23 |
| | Light | Mean ± SD | 2.7 ± 2.2 | 2.0 ± 1.3 | 3.0 ± 3.0 | 2.5 ± 2.0 | 2.6 ± 2.2 |
| | (days) | Min-Max | 0-9 | 0-5 | 1-12 | 0-7 | 0-12 |
| | Normal | Mean ± SD | 2.1 ± 1.4 | 2.5 ± 1.5 | 2.0 ± 1.1 | 1.9 ± 1.2 | 2.1 ± 1.3 |
| | (days) | Min-Max | 0-5 | 1-8 | 0-4 | 0-5 | 0-8 |
| | Heavy | Mean ± SD | 0.4 ± 0.8 | 0.3 ± 0.7 | 0.5 ± 0.9 | 0.8 ± 1.0 | 0.5 ± 0.9 |
| | (days) | Min-Max | 0-3 | 0-2 | 0-3 | 0-4 | 0-4 |
| | Number of | Mean ± SD | 1.5 ± 0.6 | 1.4 ± 0.6 | 1.8 ± 0.7 | 1.5 ± 0.6 | 1.6 ± 0.6 |
| | episodes | Min-Max | 1-3 | 1-3 | 1-3 | 1-3 | 1-3 |
| TC 4 | None | Mean ± SD | 24.1 ± 3.7 | 22.7 ± 5.3 | 24.3 ± 4.0 | 23.4 ± 5.8 | 23.6 ± 4.8 |
| | (days) | Min-Max | 17-28 | 6-28 | 9-28 | 0-28 | 0-28 |
| | Spotting | Mean ± SD | 2.0 ± 2.4 | 2.6 ± 2.5 | 2.3 ± 3.5 | 2.0 ± 2.6 | 2.2 ± 2.7 |
| | (days) | Min-Max | 0-9 | 0-8 | 0-16 | 0-9 | 0-16 |
| | Light | Mean ± SD | 1.6 ± 2.0 | 2.0 ± 2.4 | 1.1 ± 1.2 | 0.8 ± 0.9 | 1.4 ± 1.8 |
| | (days) | Min-Max | 0-7 | 0-8 | 0-4 | 0-3 | 0-8 |
| | Normal | Mean ± SD | 0.3 ± 0.9 | 0.4 ± 0.9 | 0.1 ± 0.3 | 0.4 ± 0.6 | 0.3 ± 0.7 |
| | (days) | Min-Max | 0-4 | 0-3 | 0-1 | 0-2 | 0-4 |
| | Heavy | Mean ± SD | 0 | 0.3 ± 1.1 | 0 | 0.0 ± 0.2 | 0.1 ± 0.5 |
| | (days) | Min-Max | 0 | 0-5 | 0 | 0-1 | 0-5 |
| | Number of | Mean ± SD | 1.2 ± 0.9 | 1.5 ± 1.3 | 1.4 ± 0.8 | 1.3 ± 0.8 | 1.4 ± 1.0 |
| | episodes | Min-Max | 0-3 | 0-6 | 0-3 | 0-3 | 0-6 |
| TC | None | Mean ± SD | 67.4 ± 18.0 | 65.7 ± 21.1 | 55.7 ± 20.1 | 54.8 ± 27.0 | 60.9 ± 22.2 |
| 1-3 | (days) | Min-Max | 20-87 | 0-87 | 12-91 | 1-87 | 0-91 |
| | Spotting | Mean ± SD | 11.1 ± 11.1 | 15.6 ± 16.0 | 20.4 ± 14.1 | 22.0 ± 20.2 | 17.2 ± 16.0 |
| | (days) | Min-Max | 0-51 | 0-61 | 0-48 | 0-64 | 0-64 |
| | Light | Mean ± SD | 5.6 ± 4.2 | 4.5 ± 4.1 | 7.5 ± 6.9 | 5.7 ± 5.4 | 5.8 ± 5.3 |
| | (days) | Min-Max | 1-15 | 0-19 | 1-21 | 0-24 | 0-24 |
| | Normal | Mean ± SD | 3.0 ± 2.0 | 3.5 ± 2.5 | 2.5 ± 1.5 | 2.9 ± 2.0 | 3.0 ± 2.0 |
| | (days) | Min-Max | 0-7 | 1-12 | 0-5 | 1-7 | 0-12 |
| | Heavy | Mean ± SD | 0.4 ± 0.9 | 0.5 ± 1.0 | 0.5 ± 0.9 | 1.8 ± 3.6 | 0.8 ± 2.0 |
| | (days) | Min-Max | 0-3 | 0-3 | 0-3 | 0-16 | 0-16 |
| | Number of | Mean ± SD | 3.8 ± 1.9 | 3.8 ± 2.1 | 4.3 ± 2.1 | 3.3 ± 1.6 | 3.8 ± 1.9 |
| | episodes | Min-Max | 1-8 | 1-7 | 1-12 | 1-6 | 1-12 |

Abbreviations:
DNG = dienogest;
EE = ethinyl estradiol;
Max = maximum;
Min = minimum;
N = number of subjects;
T1 = EE/DNG 10 μg/1 mg;
T2 = EE/DNG 10 μg/2 mg;
T3 = EE/DNG 20 μg/2 mg;
TC = treatment cycle;
Velmari ® = Velmari Langzyklus 0.02/3 mg tablets (EE 20 μg/drospirenone 3 mg)
¹Mean and SD values are rounded to 1 decimal place

Differences between treatment groups were largest for the categories “no bleeding” and “spotting.” Intergroup differences for “light,” “normal,” and “heavy” bleeding differed by no more than 1 day per treatment cycle.

In TC4, the number of bleeding free days was very similar between all groups. The number of bleeding events classified under “light”, “normal” and “heavy” for T3 were equal or lower than T1 or T2. Only the number of “spotting” events was higher in T3 than T1. Finally, the number of episodes differed only in 0.2 between T1 and T3. Thus, it may be concluded that in TC4 there were no significant differences between the prolonged release formulations.

During the TC1-TC3 period, a slightly more favorable profile was observed for the PR formulations with respect to the number of “heavy” bleeding, as well as for “spotting” and “no bleeding” days.

As a conclusion, with respect to the bleeding pattern no significant differences were found between the different doses of the PR formulations and these have a more favorable profile than the reference product, especially during TC1-TC3.

### Safety and Tolerability

All of the extended-release formations were found to be safe and reasonably tolerable. More than four-fifth of subjects experienced at least one product-related adverse event (AE), most of mild or moderate intensity. Headache, lower abdominal pain, and breast discomfort were the most frequent product-related AEs. The single serious AE reported in this study was unrelated to the investigation medicinal product (IMP) and occurred under Velmari® treatment. The safety data did not reveal any clinically meaningful differences between treatment groups. Details are summarized in Table 24

TABLE 24
Summary of Adverse Events for DNG-EE extended-release
formulations versus commercial DRSP-EE formulation

| | | | | | | | Velmari ® | |
|---|---|---|---|---|---|---|---|---|
| | T1 N = 25 | | T2 N = 25 | | T3 N = 25 | | N = 25 | |
| Event | n (%) | Events | n (%) | Events | n (%) | Events | n (%) | Events |
| All AE | 25 | 173 | 24 | 144 | 25 | 141 | 25 | 153 |
| | (100.0) | | (96.0) | | (100.0) | | (100.0) | |
| Pretreatment | 0 | 0 | 0 | 0 | 1 | 1 | 1 | 1 |
| AE | (0.0) | | (0.0) | | (4.0) | | (4.0) | |
| TEAE | 25 | 173 | 24 | 144 | 25 | 140 | 25 | 152 |
| | (100.0) | | (96.0) | | (100.0) | | (100.0) | |
| ADR | 22 | 94 | 20 | 67 | 21 | 70 | 21 | 72 |
| | (88.0) | | (80.0) | | (84.0) | | (84.0) | |
| Serious | 0 | 0 | 0 | 0 | 0 | 0 | 1 | 1 |
| TEAE | (0.0) | | (0.0) | | (0.0) | | (4.0) | |
| Serious ADR | 0 | 0 | 0 | 0 | 0 | 0 | 0 | 0 |
| | (0.0) | | (0.0) | | (0.0) | | (0.0) | |
| TEAE | 2 | 2 | 1 | 1 | 1 | 1 | 3 | 4 |
| leading to | (8.0) | | (4.0) | | (4.0) | | (12.0) | |
| IMP | | | | | | | | |
| withdrawal | | | | | | | | |

### Example 9: Tolerability of DNG-EE Extended-Release Formulations Versus DRSP-EE Formulations (Bleeding Patterns)

This trial was a prospective, multicentre, randomised, double-blind, double-dummy trial in female subjects who presented to the clinic seeking contraception and who were postmenarcheal and premenopausal.

### Objective

The objective was to demonstrate the safety and tolerability of Formulation 17 (Example 2, dienogest (DNG) 2 mg/ethinyl estradiol (EE) 0.02 mg) in comparison to Velmari® Langzyklus 0.02/3 mg tablets (drospirenone (DRSP) 3 mg/ethinyl estradiol (EE) 0.02 mg), especially regarding bleeding pattern.

### Materials and Methods

Film-coated tablets of each of the respective contraceptive formulations were administered orally. Each blister contained 28 tablets. One treatment cycle consisted of 24 active tablets followed by 4 placebo tablets per cycle. A total of 9 sequential cycles of 28 days corresponding to 252 days in total.

The tolerability analyses were focused on bleeding pattern changes. A trend towards less bleeding/spotting was observed with long term administration of both Formulation 17 and Velmari®.

Percentage of Subjects with Any Bleeding/Spotting Days

As summarized in Table 25, during Cycles 2-9, 90.0% of subjects reported any bleeding/spotting days in the DNG/EE group vs. 93.8% in the DRSP/EE group. The proportion of subjects with any bleeding/spotting days decreased from 87.6% (Cycle 1) to 79.9% (Cycle 9) in the DNG/EE group and from 93.0% (Cycle 2) to 86.4% (Cycle 9) in the DRSP/EE group. In the DNG/EE group, the proportions of subjects with any bleeding/spotting days and any bleeding days taken alone were lower at each reference period and overall compared to the DRSP/EE group; whereas the proportions of subjects with any spotting days were similar in both treatment groups at each defined period.

TABLE 25
% of Subjects with Any Bleeding/Spotting Days

| | % of subjects | % of subjects | % of subjects |
|---|---|---|---|
| | with bleeding | with bleeding/ | with bleeding/ |
| | days Cycles | spotting days | spotting days |
| | 2-9 | Cycle 2 | Cycle 9 |
| DNG/EE | 90.0 | 87.6 | 79.9 |
| DRSP/EE | 93.8 | 93.0 | 86.4 |

As summarized in Table 26, the proportion of subjects with any bleeding/spotting days decreased from 88.6% (Cycles 2-4) to 82.4% (Cycles 8-9) in the DNG/EE group and from 94.2% (Cycles 2-4) to 87.1% (Cycles 8-9) in the DRSP/EE group.

TABLE 26
% of Subjects with Any Bleeding/Spotting Days

| | % of subjects with | % of subjects with |
|---|---|---|
| | bleeding/spotting | bleeding/spotting |
| | days Cycles 2-4 | days Cycles 8-9 |
| DNG/EE | 88.6 | 82.4 |
| DRSP/EE | 94.2 | 87.1 |

### Mean (SD) Number of Bleeding/Spotting Days

As summarized in Table 27, the mean (SD) number of bleeding/spotting days was significantly lower in the DNG/EE group compared to the DRSP/EE group during Cycles 2-6 (13.5 [10.96] days vs. 16.7 [11.72] days; p-value: 0.0001) and Cycles 2-9 (18.8 [16.33] days vs. 23.1 [16.91] days; p-value: 0.0002).

TABLE 27
Mean (SD) Number of Bleeding/Spotting Days

| | Mean (SD) | | Mean (SD) | |
|---|---|---|---|---|
| | number | | number | |
| | Bleeding/ | | Bleeding/ | |
| | spotting days | | spotting days | |
| | Cycles 2-6 | p-value | Cycles 2-9 | p-value |
| DNG/EE | 13.5 [10.96] | 0.0001 | 18.8 [16.33] | 0.0002 |
| DRSP/EE | 16.7 [11.72] | | 23.1 [16.91] | |

As summarized in Table 28, the mean (SD) number of bleeding/spotting days decreased from 6.9 (6.15) days (Cycle 1) to 3.4 (3.05) days (Cycle 9) in the DNG/EE and from 6.1 (6.11) days (Cycle 1) to 4.3 (2.94) days (Cycle 9) in the DRSP/EE group. Among these bleeding days prevailed: The mean (SD) number of bleeding days decreased from 4.7 (4.66) days (Cycle 1) to 2.4 (2.33) days (Cycle 9) in the DNG/EE group and from 4.3 (4.60) days (Cycle 1) to 3.3 (2.35) days (Cycle 9) in the DRSP/EE group. The mean (SD) number of spotting days decreased from 2.2 (3.23) days (Cycle 1) to 1.2 (1.87) days (Cycle 3) in the DNG/EE group and from 1.8 (3.16) days (Cycle 1) to 1.1 (1.41) days (Cycle 3) in the DRSP/EE group with no major changes until Cycle 9 in both treatment groups.

TABLE 28
Mean (SD) Number of Bleeding/Spotting Days

| | | | Mean | Mean | Mean | Mean | Mean |
|---|---|---|---|---|---|---|---|
| | Mean (SD) | Mean (SD) | (SD) | (SD) | (SD) | (SD) | (SD) |
| | number | number | number | number | number | number | number |
| | Bleeding/spotting | Bleeding/spotting | bleeding | bleeding | spotting | spotting | spotting |
| | days | days | days | days | days | days | days |
| | Cycle 1 | Cycle 9 | Cycle 1 | Cycle 9 | Cycle 1 | Cycle 3 | Cycle 9 |
| DNG/EE | 6.9 | 3.4 | 4.7 | 2.4 | 2.2 | 1.2 | No |
| | (6.15) | (3.05) | (4.66) | (2.33) | (3.23) | (1.87) | changes |
| DRSP/EE | 6.1 | 4.3 | 4.3 | 3.3 | 1.8 | 1.1 | No |
| | (6.11) | (2.94) | (4.60) | (2.35) | (3.16) | (1.41) | changes |

Percentage of Subjects with Unscheduled Bleeding/Spotting Days

As summarized in Table 29, during Cycles 2-9, the proportion of subjects with unscheduled bleeding/spotting days was significantly lower (p-value: ≤0.0001) in the DNG/EE group than in the DRSP/EE group (55.1% vs. 75.2%). The proportion of subjects with unscheduled bleeding/spotting during Cycles 2-6 was lower in the DNG/EE than in the DRSP/EE group (50.5% vs. 72.8%) with a statistically significant difference between the treatment groups (95% Cl: 0.159-0.286, p-value: ≤0.0001). The non-inferiority of the DNG/EE formulation with regard to unscheduled bleeding/spotting in Cycles 2-6 compared to DRSP/EE was proved. As the confidence interval does not include zero, the superiority the DNG/EE formulation can be concluded.

TABLE 29
% of Subjects with Unscheduled Bleeding/Spotting Days

| | % of subjects | | % of subjects | |
|---|---|---|---|---|
| | with unscheduled | | with unscheduled | |
| | bleeding/spotting | | bleeding/spotting | |
| | Cycles 2-9 | p-value | Cycles 2-6 | p-value |
| DNG/EE | 55.1 | <0.0001 | 50.5 | <0.0001 |
| DRSP/EE | 75.2 | | 72.8 | |

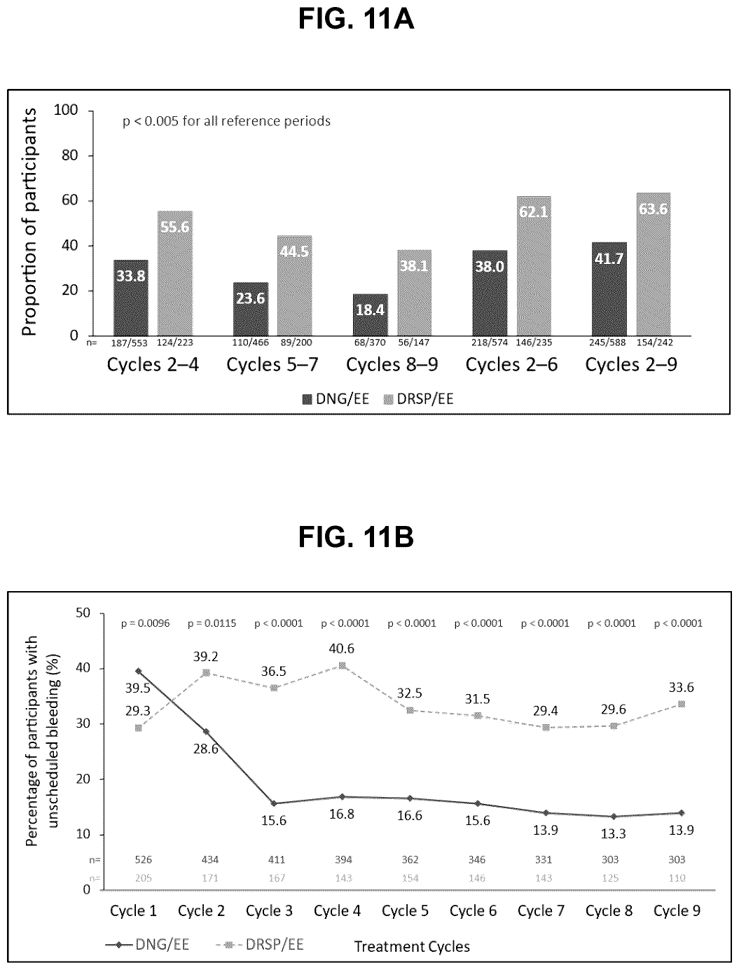

As illustrated in FIG. 11A, the proportion of participants who experienced unscheduled bleeding during Cycles 2-6 showed a similar pattern (DNG/EE, 38.0% versus DRSP/EE, 62.1%; p≤0.0001; post hoc analysis).

A similar trend was observed for participants who experienced unscheduled bleeding during Cycles 2-9, with a significantly smaller proportion of participants in the DNG/EE group experiencing unscheduled bleeding during each cycle, see FIG. 11B. Overall, the proportion of participants who experienced unscheduled bleeding in Cycles 2-9 was 41.7% (254/588) and 63.9% (154/242) for the DNG/EE and DRSP/EE groups, respectively. The proportion of participants with unscheduled bleeding/spotting during Cycles 2-9 was also significantly lower with DNG/EE versus DRSP/EE (55.1% vs. 75.2%) (p<0.0001; post hoc analysis).

As summarized in Table 30, in the DNG/EE group the proportion of subjects with unscheduled bleeding/spotting days decreased from 54.9% in Cycle 1 to 24.8% in Cycle 9. In the DRSP/EE group, no clear bleeding/spotting pattern was observed: the highest proportion of subjects with unscheduled bleeding/spotting was observed in Cycle 4 (55.9%) and the lowest in Cycle 1 (42.0%). In Cycle 9, unscheduled bleeding/spotting was observed for 48.2% of subjects. For unscheduled bleeding and spotting taken alone, the same trends as for unscheduled bleeding/spotting were observed in both treatment groups. The proportions of subjects with unscheduled bleeding/spotting, bleeding and spotting days were lower in the DNG/EE group compared to DRSP/EE group during Cycles 2-9 and at each reference period. A trend towards reduced numbers of subjects with unscheduled bleeding and/or spotting days was observed over time in both treatment groups.

TABLE 30
% of Subjects with Unscheduled Bleeding/Spotting Days

| | % of subjects with | % of subjects with |
|---|---|---|
| | unscheduled bleeding/ | unscheduled bleeding/ |
| | spotting days Cycle 1 | spotting days Cycle 9 |
| DNG/EE | 54.9 | 24.8 |
| DRSP/EE | 42.0 | 48.2 |

### Mean (SD) Number of Unscheduled Bleeding/Spotting Days

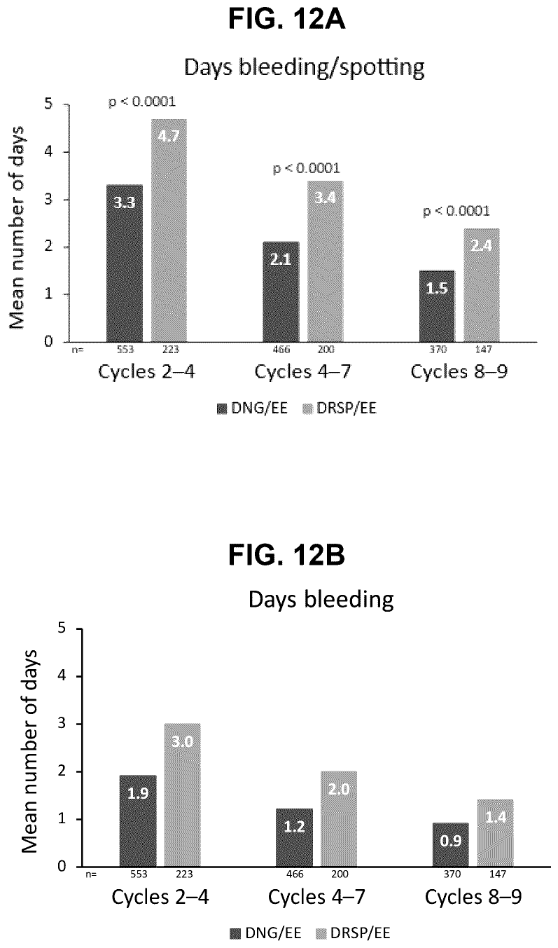

The mean number of days of unscheduled bleeding/spotting (FIG. 12A) and unscheduled bleeding (FIG. 12B) were lower with DNG/EE compared with DRSP/EE for each reference period.

Furthermore, as summarized in Table 31, during Cycles 2-9 the mean (SD) number of unscheduled bleeding/spotting days was significantly lower in the DNG/EE group than in the DRSP/EE group (5.6 [10.76] days vs. 8.7 [10.59] days; Wilcoxon-rank-sum-test p-value<0.0001).

TABLE 31
Mean (SD) Number of Unscheduled Bleeding/Spotting Days

| | Mean (SD) number | |
|---|---|---|
| | unscheduled | |
| | bleeding/spotting | |
| | days Cycles 2-9 | p-value |
| DNG/EE | 5.6 [10.76] | <0.0001 |
| DRSP/EE | 8.7 [10.59] | |

As summarized in Table 32, the reduction of the mean number of unscheduled bleeding days in the DNG/EE group is about 3 times higher than that in the DRSP/EE group and the reduction of the mean number of unscheduled spotting days in the DNG/EE group is twice as high than that in the DRSP/EE group: The mean (SD) number of unscheduled bleeding days decreased from 2.0 (3.54) days in Cycle 1 to 0.5 (1.71) days in Cycle 9 in the DNG/EE group and from 1.6 (3.54) days in Cycle 1 to 1.0 (1.81) days in Cycle 9 in the DRSP/EE group. The mean (SD) number of unscheduled spotting days decreased from 1.6 (2.78) days in Cycle 1 to 0.4 (1.06) days in Cycle 9 in the DNG/EE group and from 1.2 (2.77) days to 0.6 (1.21) days in Cycle 9 in the DRSP/EE group.

TABLE 32
Mean (SD) Number of Unscheduled Bleeding versus Spotting Days

| | Mean (SD) | Mean (SD) | Mean (SD) | Mean (SD) |
|---|---|---|---|---|
| | number | number | number | number |
| | unscheduled | unscheduled | unscheduled | unscheduled |
| | bleeding days | bleeding days | spotting days | spotting days |
| | Cycle 1 | Cycle 9 | Cycle 1 | Cycle 9 |
| DNG/EE | 2.0 (3.54) | 0.5 (1.71) | 1.6 (2.78) | 0.4 (1.06) |
| DRSP/EE | 1.6 (3.54) | 1.0 (1.81) | 1.2 (2.77) | 0.6 (1.21) |

Percentage of Subjects with Scheduled Bleeding/Spotting Days

As summarized in Table 33, during Cycles 2-9, similar proportions of subjects in both treatment groups reported scheduled bleeding/spotting: 86.6% in the DNG/EE and 86.0% in the DRSP/EE group. In the DNG/EE group, the proportion of subjects with scheduled bleeding/spotting decreased from 77.2% during Cycle 1 to 73.6% during Cycle 9. In the DRSP/EE group, no clear bleeding/spotting pattern was observed from Cycle 1 (77.1% of subjects) to Cycle 6 (78.1%), afterwards the proportion of subjects decreased to 69.1% in Cycle 9. The proportions of subjects with scheduled bleeding/spotting and bleeding were similar in both treatment groups for a specific reference period and overall, whereas the proportion of subjects with scheduled spotting was higher in the DNG/EE group compared to DRSP/EE. A trend towards reduced numbers of subjects with scheduled bleeding/spotting as well as bleeding or spotting days was observed over time in both treatment groups.

TABLE 33
% of Subjects with Scheduled Bleeding/Spotting Days

| | % of subjects | % of subjects | % of subjects |
|---|---|---|---|
| | with scheduled | with scheduled | with scheduled |
| | bleeding/spotting | bleeding/spotting | bleeding/spotting |
| | Cycles 2-9 | Cycle 1 | Cycle 9 |
| DNG/EE | 86.6 | 77.2 | 73.6 |
| DRSP/EE | 86.0 | 77.1 | 69.1 |

### Mean (SD) Number of Scheduled Bleeding and Spotting Days

As summarized in Table 34, at all cycles (except Cycle 1) and defined periods, the mean number of scheduled bleeding days was slightly lower in the DNG/EE group compared to the DRSP/EE group. From Cycle 1 to Cycle 9, the mean (SD) number of scheduled bleeding days slightly decreased from 2.7 (2.61) to 1.9 (1.77) days in the DNG/EE group and from 2.7 (2.51) days to 2.3 (1.95) days in the DRSP/EE group. The changes of mean (SD) scheduled spotting days in both groups were minimal overtime: from 0.6 (1.09) days during Cycle 1 to 0.7 (1.00) days during Cycle 9 in the DNG/EE group and from 0.6 (0.93) days to 0.4 (0.95) days, respectively, in the DRSP/EE group.

TABLE 34
Mean (SD) Number of Scheduled Bleeding versus Spotting Days

| | Mean (SD) | Mean (SD) | Mean (SD) | Mean (SD) |
|---|---|---|---|---|
| | number | number | number | number |
| | scheduled | scheduled | scheduled | scheduled |
| | bleeding days | bleeding days | spotting days | spotting days |
| | Cycle 1 | Cycle 9 | Cycle 1 | Cycle 9 |
| DNG/EE | 2.7 (2.61) | 1.9 (1.77) | 0.6 (1.09) | 0.7 (1.00) |
| DRSP/EE | 2.7 (2.51) | 2.3 (1.95) | 0.6 (0.93) | 0.4 (0.95) |

Percentage of Subjects with Frequent or Irregular Bleeding/Spotting Episodes

As summarized in Table 35, during Cycles 2-9, similar proportions of subjects in both treatment groups reported frequent bleeding/spotting episodes: 48.1% in the DNG/EE group and 47.5% in the DRSP/EE group. The proportions of subjects with frequent bleeding/spotting episodes were similar during the first 2 reference periods (ranging from 3.0% to 3.6%) in both treatment groups. Six or more bleeding/spotting episodes during the third 2-cycle reference period (Cycles 8-9) were reported by 1 (0.3%) subject in the DGN/EE group only.

As also shown in Table 35, during Cycles 2-9, the proportion of subjects with irregular bleeding/spotting was slightly higher in the DNG/EE group (50.4%) compared to the DRSP/EE group (46.4%). The highest proportion of subjects with irregular bleeding/spotting was reported during Cycles 5-7 (DNG/EE: 29.0%; DRSP/EE: 27.4%) and the lowest during Cycles 8-9 (DNG/EE: 13.8%; DRSP/EE: 14.3%) in both treatment groups.

TABLE 35
% of Subjects with Frequent or Irregular
Bleeding/Spotting Episodes

| | Frequent bleeding/ | Irregular bleeding/ |
|---|---|---|
| | spotting episodes | spotting episodes |
| | Cycles 2-9 | Cycles 2-9 |
| DNG/EE | 48.1 | 50.4 |
| DRSP/EE | 47.5 | 46.4 |

Percentage of Subjects with Prolonged Bleeding

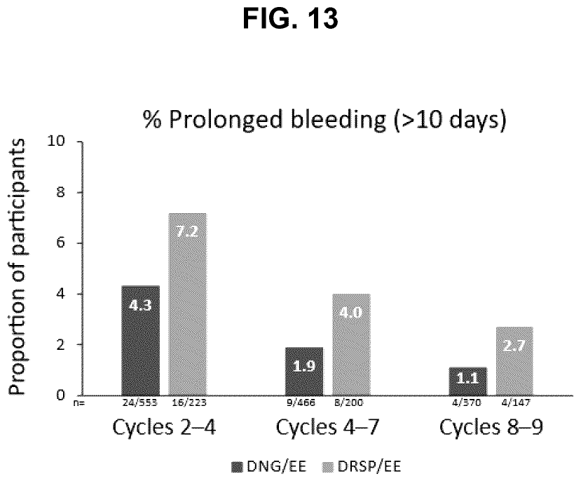

As summarized in Table 36, during Cycles 2-9, the proportion of subjects with prolonged bleeding lasting more than 10 consecutive days during the reference period was statistically significantly lower in the DNG/EE group than in the DRSP/EE group (5.6% vs. 10. 3%; p-value: 0.0154). The proportion of subjects with prolonged bleeding/spotting decreased over time from 4.3% during Cycles 2-4 to 1.1% during Cycles 8-9 in the ENG/EE group and from 7.2% to 2.7% in the DRSP/EE group (FIG. 13).

TABLE 36
% of Subjects with Prolonged Bleeding

| | Prolonged bleeding | |
|---|---|---|
| | (>10 consec. days) | p-value |
| DNG/EE | 5.6 | 0.0154 |
| DRSP/EE | 10.3 | |

Percentage of Subjects with Absence of Bleeding/Spotting

As summarized in Table 37, during Cycles 2-9, the proportion of subjects with absence of bleeding/spotting days was slightly higher in the DNG/EE group compared to the DRSP/EE group (10.5% vs 7.9%). This was also true for each reference period. A trend towards increased numbers of subjects with no bleeding/spotting days was observed over time in both treatment groups. From Cycle 1 to Cycle 9 the proportion of subjects with no bleeding/spotting increased from 12.4% to 27.4% in the DNG/EE and from 17.1% to 25.5% in the DRSP/EE group.

TABLE 37
% of Subjects with Absence of Bleeding/Spotting

| | % of subjects | % of subjects | % of subjects |
|---|---|---|---|
| | with absence of | with absence of | with absence of |
| | bleeding/spotting | bleeding/spotting | bleeding/spotting |
| | Cycles 2-9 | Cycle 1 | Cycle 9 |
| DNG/EE | 10.5 | 12.4 | 27.4 |
| DRSP/EE | 7.9 | 17.1 | 25.5 |

### Mean (SD) Number and Duration of Bleeding/Spotting Episodes

As summarized in Table 38, during Cycles 2-9, the mean (SD) number of bleeding/spotting episodes was similar in the DNG/EE and DRSP/EE groups (5.2 [3.56] vs. 5.3 [3.31]), whereas the mean (SD) duration of bleeding/spotting episodes was slightly shorter in the DNG group compared to DRSP/EE group (3.5 [2.28] days vs. 4.1 [2.68] days).

TABLE 38
Mean (SD) Number and Duration of Bleeding/Spotting Episodes

| | Mean (SD) number | Mean (SD) duration |
|---|---|---|
| | of bleeding/spotting | of bleeding/spotting |
| | episodes Cycles 2-9 | episodes Cycles 2-9 |
| DGN/EE | 5.2 [3.56] | 3.5 [2.28] |
| DRSP/EE | 5.3 [3.31] | 4.1 [2.68] |

### Premature Discontinuation Due to Metrorrhagia and Dysmenorrhoea

As summarized in Table 39, overall, 12 subjects (1.7%) discontinued prematurely due to metrorrhagia and 3 subjects (0.4%) due to dysmenorrhoea in the DNG/EE group vs. 2 subjects (0.7%) and 1 subject (0.3%) respectively in the DRSP/EE group.

TABLE 39
Discontinuations Due to Metrorragia and Dysmenorrhoea

| | Premature discontinuation | Premature discontinuation |
|---|---|---|
| | due metrorrhagia | due dysmenorrhoea |
| DNG/EE | 12 (1.7%) | 2 (0.7%) |
| DRSP/EE | 3 (0.4%) | 1 (0.3%) |

### Investigational Medicinal Product (IMP) Data

The investigational medicinal product (IMP) acceptability data collected via the questionnaire confirm that a majority of DNG/EE group (87.0%) and DRSP/EE group (89.2%) subjects strongly agreed or agreed to be satisfied with the contraception method. 11.4% of the switchers to DNP/EEQ-rated their well-being as “worse” compared to the time when they used another oral contraceptive vs. 7.1% of the switchers to DRSP/EE.

### Quality of Life Enjoyment and Satisfaction Questionnaire Short Form (Q-LES-Q-SF) Results

At baseline, mean (SD) total Quality of Life Enjoyment and Satisfaction Questionnaire Short Form (Q-LES-Q-SF) percentage scores were similar in both treatment groups: 80.69 (12.210)% in the DNG/EE and of 79.38 (11.343)% in the DRSP/EE group, with minimal changes throughout the study.

CONCLUSION: The results of this trial show that the use of DNG/EE over 9 treatment cycles provided effective contraceptive protection with a favourable bleeding pattern and was well-tolerated. In particular, the treatment with DNG/EE resulted in significantly lower frequency of unscheduled bleeding (i.e. provided better cycle control) both during Cycles 2-6 and Cycles 2-9 than the treatment with DRSP/EE. During Cycles 2-6, the treatment with DNG/EE was superior to the treatment with DRSP/EE with regard to the proportion of subjects with unscheduled bleeding. In the DNG/EE group, the reduction of the mean number of unscheduled bleeding days is about 3 times higher and the reduction of unscheduled spotting days twice as high from Cycle 1 to Cycle 9 than the respective reduction in the DRSP/EE group.

### EMBODIMENTS

Embodiment [1]: A method for oral contraception in a female subject in need thereof, the method comprising: (i) a first phase wherein active daily dosage units of an oral contraceptive composition are administered to the female subject over a period of 21 to 27 consecutive days; and (ii) a second phase wherein no contraceptive composition is administered to the female subject over a period of 1 to 7 consecutive days, wherein: the oral contraceptive composition comprises 2 mg of 17α-cyanomethyl-17-β-hydroxyestra-4,9-dien-3-one (dienogest, DNG) and 0.02 mg of 17α-ethinylestradiol (ethynyl estradiol, EE), wherein at least one of the DNG and the EE are subject to controlled release; and protection against pregnancy is not reduced when administration of the scheduled active daily oral dosage units is missed, skipped or delayed for up to 24 hours from the scheduled daily administration.

Embodiment [2]: The method of Embodiment [1], wherein the first phase lasts from 21 to 24 consecutive days, and the second phase lasts from 4 to 7 consecutive days.

Embodiment [3]: The method of Embodiment [1] or [2], wherein the first phase lasts 24 consecutive days, and the second phase lasts from 4 consecutive days.

Embodiment [4]: The method of at least one of Embodiments [1]-[3], wherein administration of one scheduled daily oral dose is skipped or delayed for up to 24 hours from the scheduled daily administration.

Embodiment [5]: The method of at least one of Embodiments [1]-[4], wherein two daily oral doses are administered within 24 hours from the scheduled daily administration of the one scheduled daily oral dose that is skipped or delayed.

Embodiment [6]: The method of at least one of Embodiments [1]-[5], wherein administration of two non-consecutive scheduled daily oral doses is skipped or delayed for up to 24 hours from the scheduled daily administration.

Embodiment [7]: The method of at least one of Embodiments [1]-[6], wherein two daily oral doses are administered within 24 hours from each scheduled daily administration of the two non-consecutive scheduled daily oral doses that are delayed or skipped.

Embodiment [8]: The method of at least one of Embodiments [1]-[7], wherein the overall Pearl Index of contraception is less than 0.7 with a confidence interval (CI) of at least 95%, wherein the overall Pearl Index is calculated as the number of confirmed on-drug pregnancies divided by cumulative cycles of exposure with the quotient multiplied by 1300.

Embodiment [9]: The method of at least one of Embodiments [1]-[8], wherein the method provides a reduced overall Pearl Index (PI) of contraception compared to orally administering a progestogen-only contraceptive composition.

Embodiment [10]: The method of at least one of Embodiments [1]-[9], wherein the method provides a reduced overall Pearl Index (PI) of contraception compared to orally administering a contraceptive composition comprising 3 mg of 17β3-Hydroxy-6β,7β:15β3,16β-dimethylene-3-oxo-17α-pregn-4-ene-21-carboxylic acid, γ-lactone (drospirenone, DRSP) and 0.02 mg of 17α-ethinylestradiol (ethynyl estradiol, EE), wherein the overall Pearl Index is calculated as the number of confirmed on-drug pregnancies divided by cumulative cycles of exposure with the quotient multiplied by 1300.

Embodiment [11]: The method of at least one of Embodiments [1]-[10], wherein the method provides a reduced overall Pearl Index (PI) of contraception compared to orally administering a contraceptive composition comprising 2 mg of 17α-cyanomethyl-17-β-hydroxyestra-4,9-dien-3-one (dienogest, DNG) and 0.03 mg of 17α-ethinylestradiol (ethynyl estradiol, EE), wherein the overall Pearl Index is calculated as the number of confirmed on-drug pregnancies divided by cumulative cycles of exposure with the quotient multiplied by 1300.

Embodiment [12]: The method of at least one of Embodiments [1]-[11], wherein the overall Pearl Index of contraception is less than 4, with a confidence interval (CI) of at least 95%, when the method is carried out over a period of 7 to 9 menstruation cycles, wherein the overall Pearl Index is calculated as the number of confirmed on-drug pregnancies divided by cumulative cycles of exposure with the quotient multiplied by 1300.

Embodiment [13]: The method of at least one of Embodiments [1]-[12], wherein the overall Pearl Index of contraception is less than 2, with a confidence interval (CI) of at least 95%, when the method is carried out over a period of 7 to 9 menstruation cycles, wherein the overall Pearl Index is calculated as the number of confirmed on-drug pregnancies divided by cumulative cycles of exposure with the quotient multiplied by 1300.

Embodiment [14]: The method of at least one of Embodiments [1]-[13], wherein the overall Pearl Index of contraception is less than 0.7, with a confidence interval (CI) of at least 95%, when the method is carried out over a period of 7 to 9 menstruation cycles, wherein the overall Pearl Index is calculated as the number of confirmed on-drug pregnancies divided by cumulative cycles of exposure with the quotient multiplied by 1300.

Embodiment [15]: The method of at least one of Embodiments [1]-[14], wherein the overall Pearl Index of contraception is less than 0.6, with a confidence interval (CI) of 95%, wherein the overall Pearl Index is calculated as the number of confirmed on-drug pregnancies divided by cumulative cycles of exposure with the quotient multiplied by 1300.

Embodiment [16]: The method of at least one of Embodiments [1]-[15], wherein the overall Pearl Index of contraception is less than 0.4, with a confidence interval (CI) of 95%, wherein the overall Pearl Index is calculated as the number of confirmed on-drug pregnancies divided by cumulative cycles of exposure with the quotient multiplied by 1300.

Embodiment [17]: The method of at least one of Embodiments [1]-[16], wherein the overall Pearl Index of contraception is less than or equal to 0.2, with a confidence interval (CI) of 95%, wherein the overall Pearl Index is calculated as the number of confirmed on-drug pregnancies divided by cumulative cycles of exposure with the quotient multiplied by 1300.

Embodiment [18]: The method of at least one of Embodiments [1]-[17], wherein the method provides an improved bleeding profile in the female subject compared to orally administering a progestogen-only contraceptive composition.

Embodiment [19]: The method of at least one of Embodiments [1]-[18], wherein the method provides an improved bleeding profile in the female subject compared to orally administering a contraceptive composition comprising 3 mg of 17β3-Hydroxy-6β,7β:15β,16β-dimethylene-3-oxo-17α-pregn-4-ene-21-carboxylic acid, γ-lactone (drospirenone, DRSP) and 0.02 mg of 17α-ethinylestradiol (ethynyl estradiol, EE).

Embodiment [20]: The method of at least one of Embodiments [1]-[19], wherein the method provides an improved bleeding profile in the female subject compared to orally administering a contraceptive composition comprising 2 mg of 17α-cyanomethyl-17-β-hydroxyestra-4,9-dien-3-one (dienogest, DNG) and 0.03 mg of 17α-ethinylestradiol (ethynyl estradiol, EE).

Embodiment [21]: The method of at least one of Embodiments [1]-[20], wherein the improved bleeding profile comprises reduced incidences of spotting and/or bleeding.

Embodiment [22]: The method of at least one of Embodiments [1]-[21], wherein, when the oral contraceptive composition is administered to a plurality of female subjects over a period of at least 9 menstruation cycles, no more than 90% of the female subjects experience bleeding, spotting, or a combination thereof, during menstruation cycles 2-9.

Embodiment [23]: The method of at least one of Embodiments [1]-[22], wherein, when the oral contraceptive composition is administered to a plurality of female subjects over a period of at least 9 menstruation cycles, no more than 60% of the female subjects experience unscheduled bleeding, unscheduled spotting, or a combination thereof, during menstruation cycles 2-9.

Embodiment [24]: The method of at least one of Embodiments [1]-[23], wherein, when the oral contraceptive composition is administered to a plurality of female subjects over a period of at least 6 menstruation cycles, no more than 55% of the female subjects experience unscheduled bleeding, unscheduled spotting, or a combination thereof, during menstruation cycles 2-6.

Embodiment [25]: The method of at least one of Embodiments [1]-[24], wherein, when the oral contraceptive composition is administered to a plurality of female subjects over a period of at least 9 menstruation cycles, no more than 90% of the female subjects experience scheduled bleeding, scheduled spotting, or a combination thereof, during menstruation cycles 2-9.

Embodiment [26]: The method of at least one of Embodiments [1]-[25], wherein, when the oral contraceptive composition is administered to a plurality of female subjects over a period of at least 9 menstruation cycles, no more than 50% of the female subjects experience frequent bleeding, frequent spotting, or a combination thereof, during menstruation cycles 2-9.

Embodiment [27]: The method of at least one of Embodiments [1]-[26], wherein, when the oral contraceptive composition is administered to a plurality of female subjects over a period of at least 9 menstruation cycles, no more than 55% of the female subjects experience irregular bleeding, irregular spotting, or a combination thereof, during menstruation cycles 2-9.

Embodiment [28]: The method of at least one of Embodiments [1]-[27], wherein, when the oral contraceptive composition is administered to a plurality of female subjects over a period of at least 9 menstruation cycles, no more than 55% of the female subjects experience prolonged bleeding lasting more than ten consecutive days, prolonged spotting lasting more than ten consecutive days, or a combination thereof, during menstruation cycles 2-9.

Embodiment [29]: The method of at least one of Embodiments [1]-[28], wherein, when the oral contraceptive composition is administered to a plurality of female subjects over a period of at least 9 menstruation cycles, at least 10% of the female subjects experience absence of bleeding, absence of spotting, or a combination thereof, during menstruation cycles 2-9.

Embodiment [30]: The method of at least one of Embodiments [1]-[29], wherein the oral contraceptive composition comprises 2 mg of 17α-cyanomethyl-17-β-hydroxyestra-4,9-dien-3-one (dienogest, DNG) and 0.02 mg of 17α-ethinylestradiol (ethynyl estradiol, EE) in an extended-release matrix.

Embodiment [31]: The method of Embodiment [1]-[30], wherein the extended-release matrix comprises a diluent, a polymeric matrix, a binder, and a lubricant.

Embodiment [32]: The method of at least one of Embodiments [1]-[31], wherein the extended-release matrix is formed from a granular core comprising a polymeric matrix forming agent and a diluent.

Embodiment [33]: The method of at least one of Embodiments [1]-[32], wherein a mass ratio of the polymeric matrix forming agent to the diluent in the granular core ranges from 2:1 to 10:1.

Embodiment [34]: The method of at least one of Embodiments [1]-[33], wherein the mass ratio of the polymeric matrix forming agent to the diluent in the granular core ranges from 3:1 to 10:1, or from 3:1 to 9:1, or from 4:1 to 9:1, or from 4:1 to 8:1, or from 5:1 to 8:1, or from 5.5:1 to 8.5:1, or from 6:1 to 8:1, or from 6.5 to 8:1, or from 6.5:1 to 7:1, or from 7:1 to 8:1, or from 7:1 to 7.5:1, or from 7.5:1 to 8:1, or from 8:1 to 8.5:1.

Embodiment [35]: The method of at least one of Embodiments [1]-[34], wherein the mass ratio of the polymeric matrix forming agent to the diluent in the granular core is about 7.2:1.

Embodiment [36]: The method of at least one of Embodiments [1]-[35], wherein the extended-release matrix comprises the granular core, the DNG, the EE, and an additional amount of the diluent.

Embodiment [37]: The method of at least one of Embodiments [1]-[36], wherein the granular core further comprises a binder.

Embodiment [38]: The method of at least one of Embodiments [1]-[37], wherein the polymeric matrix is a hydroxypropyl methylcellulose (HPMC).

Embodiment [39]: The method of at least one of Embodiments [1]-[38], wherein the polymeric matrix is HPMC K100 (a hydroxypropyl methylcellulose thickener yielding a viscosity of 100,000 cP at 2% in water).

Embodiment [40]: The method of at least one of Embodiments [1]-[39], wherein the diluent is a lactose.

Embodiment [41]: The method of at least one of Embodiments [1]-[40], wherein the diluent is lactose monohydrate.

Embodiment [42]: The method of at least one of Embodiments [1]-[41], wherein the extended-release matrix comprises: (a) 30-60% w/w of a diluent based on a total weight of the composition; (b) 10-60% w/w of a polymeric matrix based on the total weight of the composition; (c) 1-10% w/w of a binder based on a total weight of the composition; and (d) 0-5% w/w of a lubricant based on a total weight of the composition.

Embodiment [43]: The method of at least one of Embodiments [1]-[42], wherein the extended-release matrix comprises: (a) 35-45% w/w of a lactose based on a total weight of the composition; (b) 45-55% w/w of a hydroxypropyl methylcellulose (HPMC) based on the total weight of the composition; (c) 2-7% w/w of a povidone based on the total weight of the composition; and (d) 1-3% w/w of a stearate based on the total weight of the composition.

Embodiment [44]: The method of at least one of Embodiments [1]-[43], wherein the extended-release matrix comprises: (a) 35-45% w/w of lactose monohydrate based on a total weight of the composition; (b) 45-55% w/w of HPMC K100 (a hydroxypropyl methylcellulose thickener yielding a viscosity of 100,000 cP at 2% in water) based on the total weight of the composition; (c) 2-7% w/w of povidone K30 (a cross-linked polyvinylpyrrolidone having average molecular weight of 40,000) based on the total weight of the composition; and (d) 1-3% w/w of a magnesium stearate based on the total weight of the composition.

Embodiment [45]: The method of at least one of Embodiments [1]-[44], wherein the extended-release matrix comprises: (a) 46.82% w/w of the HPMC K100 (a hydroxypropyl methylcellulose thickener yielding a viscosity of 100,000 cP at 2% in water) based on a total weight of the composition; (b) 36.48% w/w of the lactose monohydrate based on the total weight of the composition; (c) 4.68% w/w of the povidone K30 (a cross-linked polyvinylpyrrolidone having average molecular weight of 40,000) based on a total weight of the composition; and (d) 1.87% w/w of the magnesium stearate based on a total weight of the composition.

Embodiment [46]: The method of at least one of Embodiments [1]-[45], wherein the extended-release matrix has a Carr Index ranging from 17% to 18%.

Embodiment [47]: The method of at least one of Embodiments [1]-[46], wherein the extended-release matrix has a Carr Index ranging from 17.0% to 17.5%.

Embodiment [48]: The method of at least one of Embodiments [1]-[47], wherein the DNG and the EE are formulated such that, when the oral contraceptive composition is subjected to an in vitro dissolution test according to USP1 (baskets) method using 500 mL of water at 37° C. (±0.5° C.) at a stirring rate of 75 rpm, the DNG and the EE, respectively, exhibit a dissolution profile such that between 30% and 60% of the DNG initially present in the composition, and between 30% and 60% of the EE initially present in the composition, is dissolved within 2 hours.

Embodiment [49]: The method of at least one of Embodiments [1]-[48], wherein the DNG and the EE are formulated such that, when the oral contraceptive composition is subjected to an in vitro dissolution test according to USP1 (basket) method using 500 mL of water at 37° C. (±0.5° C.) at a stirring rate of 75 rpm, the DNG and the EE, respectively, exhibit a dissolution profile such that: (i) no more than 20% of the DNG initially present in the composition is dissolved within 0.5 hour; (ii) no more than 25% of the EE initially present in the composition is dissolved within 0.5 hour; (iii) between 30% and 60% of the DNG initially present in the composition is dissolved within 2 hours; (iv) between 30% and 60% of the EE initially present in the composition is dissolved within 2 hours; (v) at least 70% of the DNG initially present in the composition is dissolved within 8 hours; (vi) at least 70% of the EE initially present in the composition is dissolved within 8 hours; or any combination of (i)-(vi).

Embodiment [50]: The method of at least one of Embodiments [1]-[49], wherein the DNG and the EE are formulated such that, when the oral contraceptive composition is subjected to an in vitro dissolution test according to USP1 (basket) method using 500 mL of water at 37° C. (±0.5° C.) at a stirring rate of 75 rpm, the DNG and the EE, respectively, exhibit a dissolution profile such that: (i) no more than 20% of the DNG initially present in the composition is dissolved within 0.5 hour; (ii) between 5% and 25% of the EE initially present in the composition is dissolved within 0.5 hour; (iii) between 37% and 57% of the DNG initially present in the composition is dissolved within 2 hours; (iv) between 37% and 57% of the EE initially present in the composition is dissolved within 2 hours; (v) at least 80% of the DNG initially present in the composition is dissolved within 8 hours; (vi) at least 80% of the EE initially present in the composition is dissolved within 8 hours; or any combination of (i)-(vi).

Embodiment [51]: The method of at least one of Embodiments [1]-[50], wherein, after single-dose oral administration to the female subject, the oral contraceptive composition provides: a Cₘₐₓ of EE of less than 75 μg/mL; and a Cₘₐₓ of DNG of less than 70 ng/mL.

Embodiment [52]: The method of at least one of Embodiments [1]-[51], wherein after single-dose oral administration to the female subject, the oral contraceptive composition provides: a Cₘₐₓ of EE ranging from 29.2 μg/mL to 36.9 μg/mL; and a Cₘₐₓ of DNG ranging from 34.9 ng/mL to 48.5 ng/mL.

Embodiment [53]: The method of at least one of Embodiments [1]-[52], wherein, after single-dose oral administration to the female subject under fasting conditions, the oral contraceptive composition provides: a Tmax of EE ranging from 2.0 hours to 5.0 hours; and a Tmax of DNG ranging from 1.5 hours to 6.0 hours.

Embodiment [54]: The method of at least one of Embodiments [1]-[53], wherein after single-dose oral administration to the female subject, the oral contraceptive composition provides: a Tmax of EE ranging from 3.1 hours to 4.7 hours; and a Tmax of DNG ranging from 3.0 hours to 4.4 hours.

Embodiment [55]: The method of at least one of Embodiments [1]-[54], wherein, after single-dose oral administration to the female subject, the oral contraceptive composition provides: an AUC_{0-72 hr} of EE of at least 200 pg×h/mL; and an AUC_{0-72 hr} of DNG of at least 300 ng×h/mL.

Embodiment [56]: The method of at least one of Embodiments [1]-[55], wherein after single-dose oral administration to the female subject, the oral contraceptive composition provides: an AUC_{0-72 hr} of EE ranging from 435 pg×h/mL to 562 pg×h/mL; and an AUC_{0-72 hr} of DNG ranging from 545 ng×h/mL to 689 ng×h/mL.

Embodiment [57]: The method of at least one of Embodiments [1]-[56], wherein, after single-dose oral administration to the female subject, the oral contraceptive composition provides: a Cₘₐₓ of EE of less than 75 μg/mL; and a Cₘₐₓ of DNG of less than 70 ng/mL; a Tmax of EE ranging from 2.0 hours to 5.0 hours; and a Tmax of DNG ranging from 1.5 hours to 6.0 hours; and an AUC_{0-72 hr} of EE of at least 200 pg×h/mL; and an AUC_{0-72 hr} of DNG of at least 300 ng×h/mL.

Embodiment [58]: The method of at least one of Embodiments [1]-[57], wherein after single-dose oral administration to the female subject, the oral contraceptive composition provides: a Cₘₐₓ of EE ranging from 29.2 μg/mL to 36.9 μg/mL; a Tmax of EE ranging from 3.1 hours to 4.7 hours; an AUC_{0-72 hr} of EE ranging from 435 pg×h/mL to 562 pg×h/mL; a Cₘₐₓ of DNG ranging from 34.9 ng/mL to 48.5 ng/mL; a Tmax of DNG ranging from 3.0 hours to 4.4 hours; and an AUC_{0-72 hr} of DNG ranging from 545 ng×h/mL to 689 ng×h/mL.

Embodiment [59]: The method of at least one of Embodiments [1]-[58], wherein after once daily oral administration to the female subject under fasting conditions for 7 days, the oral contraceptive composition provides: a Cₘₐₓ of EE ranging from 60 μg/mL to 65 μg/mL; and a Cₘₐₓ of DNG ranging from 55 ng/mL to 60 ng/mL.

Embodiment [60]: The method of at least one of Embodiments [1]-[59], wherein after once daily oral administration to the female subject under fasting conditions for 7 days, the oral contraceptive composition provides: a Tmax of EE ranging from 3.5 hours to 4.0 hours; and a Tmax of DNG ranging from 3.5 hours to 4.0 hours.

Embodiment [61]: The method of at least one of Embodiments [1]-[60], wherein after once daily oral administration to the female subject under fasting conditions for 7 days, the oral contraceptive composition provides: an AUC_{0-24 hr} of EE ranging from 680 pg×h/mL to 710 pg×h/mL; and an AUC_{0-24 hr} of DNG ranging from 710 ng×h/mL to 740 ng×h/mL.

Embodiment [62]: The method of at least one of Embodiments [1]-[61], wherein after once daily oral administration to the female subject under fasting conditions for 7 days, the oral contraceptive composition provides: a Cₘₐₓ of EE ranging from 60 μg/mL to 65 μg/mL; a Tmax of EE ranging from 3.5 hours to 4.0 hours; an AUC_{0-24 hr} of EE ranging from 680 pg×h/mL to 710 pg×h/mL; a Cₘₐₓ of DNG ranging from 55 ng/mL to 60 ng/mL; a Tmax of DNG ranging from 3.5 hours to 4.0 hours; and an AUC_{0-24 hr} of DNG ranging from 710 ng×h/mL to 740 ng×h/mL.

Embodiment [63]: The method of at least one of Embodiments [1]-[62], wherein after single-dose oral administration to the female subject under fasting conditions, the oral contraceptive composition provides: a Cₘₐₓ of EE of less than 65 μg/mL; and a Cₘₐₓ of DNG of less than 65 ng/mL.

Embodiment [64]: The method of at least one of Embodiments [1]-[63], wherein after single-dose oral administration to the female subject under fasting conditions, the oral contraceptive composition provides: a Cₘₐₓ of EE ranging from 29.2 μg/mL to 35.6 μg/mL; and a Cₘₐₓ of DNG ranging from 34.9 ng/mL to 42.7 ng/mL.

Embodiment [65]: The method of at least one of Embodiments [1]-[64], wherein after single-dose oral administration to the female subject under fasting conditions, the oral contraceptive composition provides: a Tmax of EE ranging from 3.0 hours to 5.0 hours; and a Tmax of DNG ranging from 3.0 hours to 6.0 hours.

Embodiment [66]: The method of at least one of Embodiments [1]-[65], wherein after single-dose oral administration to the female subject under fasting conditions, the oral contraceptive composition provides: a Tmax of EE ranging from 3.9 hours to 4.7 hours; and a Tmax of DNG ranging from 3.6 hours to 4.4 hours.

Embodiment [67]: The method of at least one of Embodiments [1]-[66], wherein after single-dose oral administration to the female subject under fasting conditions, the oral contraceptive composition provides: an AUC_{0-72 hr} of EE of at least 200 pg×h/mL; and an AUC_{0-72 hr} of DNG of at least 300 ng×h/mL.

Embodiment [68]: The method of at least one of Embodiments [1]-[67], wherein after single-dose oral administration to the female subject under fasting conditions, the oral contraceptive composition provides: an AUC_{0-72 hr} of EE ranging from 435 pg×h/mL to 531 pg×h/mL; and an AUC_{0-72 hr} of DNG ranging from 545 ng×h/mL to 665 ng×h/mL.

Embodiment [69]: The method of at least one of Embodiments [1]-[68], wherein after single-dose oral administration to the female subject under fasting conditions, the oral contraceptive composition provides: a Cₘₐₓ of EE of less than 65 μg/mL; and a Cₘₐₓ of DNG of less than 65 ng/mL; a Tmax of EE ranging from 2.0 hours to 5.0 hours; and a Tmax of DNG ranging from 1.5 hours to 6.0 hours and an AUC_{0-72 hr} of EE of at least 200 pg×h/mL; and an AUC_{0-72 hr} of DNG of at least 300 ng×h/mL.

Embodiment [70]: The method of at least one of Embodiments [1]-[69], wherein after single-dose oral administration to the female subject under fasting conditions, the oral contraceptive composition provides: a Cₘₐₓ of EE ranging from 29.2 μg/mL to 35.6 μg/mL; a Tmax of EE ranging from 3.9 hours to 4.7 hours; an AUC_{0-72 hr} of EE ranging from 435 pg×h/mL to 531 pg×h/mL; a Cₘₐₓ of DNG ranging from 34.9 ng/mL to 42.7 ng/mL; a Tmax of DNG ranging from 3.6 hours to 4.4 hours; and an AUC_{0-72 hr} of DNG ranging from 545 ng×h/mL to 665 ng×h/mL.

Embodiment [71]: The method of at least one of Embodiments [1]-[70], wherein after single-dose oral administration to the female subject under fed conditions, the oral contraceptive composition provides: a Cₘₐₓ of EE of less than 60 μg/mL; and a Cₘₐₓ of DNG of less than 65 ng/mL.

Embodiment [72]: The method of at least one of Embodiments [1]-[71], wherein after single-dose oral administration to the female subject under fed conditions, the oral contraceptive composition provides: a Cₘₐₓ of EE ranging from 30.2 μg/mL to 36.9 μg/mL; and a Cₘₐₓ of DNG ranging from 40.0 ng/mL to 48.5 ng/mL.

Embodiment [73]: The method of at least one of Embodiments [1]-[72], wherein after single-dose oral administration to the female subject under fed conditions, the oral contraceptive composition provides: a Tmax of EE ranging from 2.0 hours to 5.0 hours; and a Tmax of DNG ranging from 1.5 hours to 5 hours.

Embodiment [74]: The method of at least one of Embodiments [1]-[73], wherein after single-dose oral administration to the female subject under fed conditions, the oral contraceptive composition provides: a Tmax of EE ranging from 3.4 hours to 4.1 hours; and a Tmax of DNG ranging from 3.0 hours to 3.6 hours.

Embodiment [75]: The method of at least one of Embodiments [1]-[74], wherein after single-dose oral administration to the female subject under fed conditions, the oral contraceptive composition provides: an AUC_{0-72 hr} of EE of at least 200 pg×h/mL; and an AUC_{0-72 hr} of DNG of at least 300 ng×h/mL.

Embodiment [76]: The method of at least one of Embodiments [1]-[75], wherein after single-dose oral administration to the female subject under fed conditions, the oral contraceptive composition provides: an AUC_{0-72 hr} of EE ranging from 459 pg×h/mL to 562 pg×h/mL; and an AUC_{0-72 hr} of DNG ranging from 563 ng×h/mL to 689 ng×h/mL.

Embodiment [77]: The method of at least one of Embodiments [1]-[76], wherein after single-dose oral administration to the female subject under fed conditions, the oral contraceptive composition provides: a Cₘₐₓ of EE of less than 60 μg/mL; and a Cₘₐₓ of DNG of less than 65 ng/mL; a Tmax of EE ranging from 2.0 hours to 5.0 hours; and a Tmax of DNG ranging from 1.5 hours to 5 hours; and an AUC_{0-72 hr} of EE of at least 200 pg×h/mL; and an AUC_{0-72 hr} of DNG of at least 300 ng×h/mL.

Embodiment [78]: The method of at least one of Embodiments [1]-[77], wherein after single-dose oral administration to the female subject under fed conditions, the oral contraceptive composition provides: a Cₘₐₓ of EE ranging from 30.2 μg/mL to 36.9 μg/mL; a Tmax of EE ranging from 3.4 hours to 4.1 hours; an AUC_{0-72 hr} of EE ranging from 459 pg×h/mL to 562 pg×h/mL; a Cₘₐₓ of DNG ranging from 40.0 ng/mL to 48.5 ng/mL; a Tmax of DNG ranging from 3.0 hours to 3.6 hours; and an AUC_{0-72 hr} of DNG ranging from 563 ng×h/mL to 689 ng×h/mL.

Embodiment [79]: The method of at least one of Embodiments [1]-[78], wherein the oral contraceptive composition is in the form of a tablet comprising the extended-release matrix.

Embodiment [80]: The method of at least one of Embodiments [1]-[79], wherein a hardness of the tablet ranges from 15N to 60 N.

Embodiment [81]: The method of at least one of Embodiments [1]-[80], wherein a hardness of the tablet ranges from 28N to 58 N.

Embodiment [82]: The method of at least one of Embodiments [1]-[81], wherein the tablet is a film-coated tablet comprising a tablet core and a film coating, and wherein the tablet core comprises the extended-release matrix.

Embodiment [83]: The method of at least one of Embodiments [1]-[82], wherein the film coating comprises a polymer, a plasticizer and a pigment.

Embodiment [84]: The method of at least one of Embodiments [1]-[83], wherein the polymer comprises a polyethylene glycol.

Embodiment [85]: The method of at least one of Embodiments [1]-[84], wherein the polymer comprises a polyethylene glycol having an average molecular weight within 5,000-7,000.

Embodiment [86]: The method of at least one of Embodiments [1]-[85], wherein the oral contraceptive composition does not comprise estrogen.

### REFERENCE LIST

Hoogland H J, Skouby S O. Ultrasound evaluation of ovarian activity under oral contraceptives. Contraception. 1993 June; 47(6):583-90.
Klipping C, Duijkers I, Remmers A, et al. Ovulation-inhibiting effects of dienogest in a randomized, dose-controlled pharmacodynamic trial of healthy women, J Clin Pharmacol. 2012 Nov; 52(11):1704-13.
Landgren B M, Unden A L, Diczfalusy E. Hormonal profile of the cycle in 68 normally menstruating women. Acta Endocrinol (Copenh). 1980 May; 94(1):89-98.
Nappi R. E., Lete I., Lee L. K., Flores N. M., Micheletti M. C., Tang B., Real-world experience of women using extended-cycle vs monthly-cycle combined oral contraception in the United States: the National health and Wellness Survey. BMC Women's Health 2018 18:1 Article Number 22.
Wiegratz I, Stahlberg S, Manthey T, et al. Effect of extended-cycle regimen with an oral contraceptive containing 30 mcg ethinyl estradiol and 2 mg dienogest on bleeding patterns, safety, acceptance and contraceptive efficacy. Contraception. 2011 August; 84(2):133-43.
Wiegratz I, Kutschera E, Lee J H, et al. Effect of four different oral contraceptives on various sex hormones and serum-binding globulins. Contraception. 2003; 67(1):25-32.
Wiegratz I, Lee J H, Kutschera E, Winkler U H, Kuhl H. Effect of four oral contraceptives on hemostatic parameters. Contraception. 2004; 70(2):97-106.
Helmerhorst F M, Rosendaal F R, Vandenbroucke J P. Venous thromboembolism and the pill. The WHO technical report on cardiovascular disease and steroid hormone contraception: state-of-the-art. World Health Organization. Hum Reprod. 1998; 13(11): 2981-2983.
Meade T W. Oral contraceptives, clotting factors, and thrombosis. Am J Obstet Gynecol. 1982; 142(6 Pt 2):758-761.
Gerstman B B, Piper J M, Tomita D K, Ferguson W J, Stadel B V, Lundin F E. Oral contraceptive estrogen dose and the risk of deep venous thromboembolic disease. Am J Epidemiol. 1991; 133(1):32-37.
Lidegaard O. Oral contraception and risk of a cerebral thromboembolic attack: results of a case-control study. BMJ. 1993; 306(6883):956-963.
Guida M, Bifulco G, Di Spiezio Sardo A, Scala M, Fernandez L M, Nappi C. Review of the safety, efficacy and patient acceptability of the combined dienogest/estradiol valerate contraceptive pill. Int J Womens Health. 2010; 2:279-290.
Stanczyk F Z, Archer D F, Bhavnani B R. Ethinyl estradiol and 17β-estradiol in combined oral contraceptives: pharmacokinetics, pharmacodynamics and risk assessment. Contraception. 2013; 87(6):706-727 doi:10.1016/j.contraception.2012.12.011.
Stanczyk F Z, Winer S A, Foidart J-M, et al. Comparison of estrogenic components used for hormonal contraception. Contraception. 2024; 130.
Valette. Summary of Product Characteristics. Bayer A G. Available from: https://resources.bayer.com.au/resources/uploads/P/file9439.pdf Accessed April, 2024

1. A method for oral contraception in a female subject in need thereof, the method comprising:
(i) a first phase wherein active daily dosage units of an oral contraceptive composition are administered to the female subject over a period of 24 days, with a proviso that no more than two of the 24 days may be non-consecutive; and
(ii) a second phase wherein no contraceptive composition is administered to the female subject over a period of 4 consecutive days,
wherein:
the oral contraceptive composition comprises 2 mg of 17α-cyanomethyl-17-β-hydroxyestra-4,9-dien-3-one (dienogest, DNG) and 0.02 mg of 17α-ethinylestradiol (ethynyl estradiol, EE);
the DNG and the EE are both subject to controlled release, such that after single-dose oral administration to the female subject, the oral contraceptive composition provides:
a Tmax of EE ranging from 3.1 hours to 4.7 hours, and
a Tmax of DNG ranging from 3.0 hours to 4.4 hours;
the active daily dosage units of the oral contraceptive composition are scheduled to be administered to the female subject every day at about the same time; and
administration of the scheduled active daily oral dosage units can be missed, skipped or delayed for up to 24 hours from the scheduled daily administration.

2. The method of claim 1, wherein two daily oral doses are administered within 24 hours from the scheduled daily administration of the one scheduled daily oral dose that is missed, skipped or delayed.

3. The method of claim 1, wherein administration of one or two non-consecutive scheduled daily oral doses are skipped or delayed for up to 24 hours from the scheduled daily administration.

4. The method of claim 1, wherein the overall Pearl Index of contraception is less than 0.7 with a confidence interval (CI) of at least 95%, wherein the overall Pearl Index is calculated as the number of confirmed on-drug pregnancies divided by cumulative cycles of exposure with the quotient multiplied by 1300.

5. The method of claim 1, wherein an overall Pearl Index of contraception is less than or equal to 0.2 with a confidence interval (CI) of 95%, wherein the overall Pearl Index is calculated as the number of confirmed on-drug pregnancies divided by cumulative cycles of exposure with the quotient multiplied by 1300.

6. The method of claim 1, wherein, when the oral contraceptive composition is administered to a plurality of female subjects over a period of at least 9 menstruation cycles, no more than 90% of the female subjects experience bleeding, spotting, or a combination thereof, during menstruation cycles 2-9.

7. The method of claim 1, wherein, when the oral contraceptive composition is administered to a plurality of female subjects over a period of at least 9 menstruation cycles, no more than 60% of the female subjects experience unscheduled bleeding, unscheduled spotting, or a combination thereof, during menstruation cycles 2-9.

8. The method of claim 1, wherein, when the oral contraceptive composition is administered to a plurality of female subjects over a period of at least 6 menstruation cycles, no more than 55% of the female subjects experience unscheduled bleeding, unscheduled spotting, or a combination thereof, during menstruation cycles 2-6.

9. The method of claim 1, wherein, when the oral contraceptive composition is administered to a plurality of female subjects over a period of at least 9 menstruation cycles, no more than 90% of the female subjects experience scheduled bleeding, scheduled spotting, or a combination thereof, during menstruation cycles 2-9.

10. The method of claim 1, wherein, when the oral contraceptive composition is administered to a plurality of female subjects over a period of at least 9 menstruation cycles, no more than 50% of the female subjects experience frequent bleeding, frequent spotting, or a combination thereof, during menstruation cycles 2-9.

11. The method of claim 1, wherein, when the oral contraceptive composition is administered to a plurality of female subjects over a period of at least 9 menstruation cycles, no more than 55% of the female subjects experience irregular bleeding, irregular spotting, or a combination thereof, during menstruation cycles 2-9.

12. The method of claim 1, wherein, when the oral contraceptive composition is administered to a plurality of female subjects over a period of at least 9 menstruation cycles, no more than 55% of the female subjects experience prolonged bleeding lasting more than ten consecutive days, prolonged spotting lasting more than ten consecutive days, or a combination thereof, during menstruation cycles 2-9.

13. The method of claim 1, wherein, when the oral contraceptive composition is administered to a plurality of female subjects over a period of at least 9 menstruation cycles, at least 10% of the female subjects experience absence of bleeding, absence of spotting, or a combination thereof, during menstruation cycles 2-9.

14. The method of claim 1, wherein the DNG and the EE are formulated such that, when the oral contraceptive composition is subjected to an in vitro dissolution test according to USP1 (baskets) method using 500 mL of water at 37° C. (±0.5° C.) at a stirring rate of 75 rpm, the DNG and the EE, respectively, exhibit a dissolution profile such that between 30% and 60% of the DNG initially present in the composition, and between 30% and 60% of the EE initially present in the composition, is dissolved within 2 hours.

15. The method of claim 1, wherein the DNG and the EE are formulated such that, when the oral contraceptive composition is subjected to an in vitro dissolution test according to USP1 (basket) method using 500 mL of water at 37° C. (±0.5° C.) at a stirring rate of 75 rpm, the DNG and the EE, respectively, exhibit a dissolution profile such that:
(i) no more than 20% of the DNG initially present in the composition is dissolved within 0.5 hour;
(ii) no more than 25% of the EE initially present in the composition is dissolved within 0.5 hour;
(iii) between 30% and 60% of the DNG initially present in the composition is dissolved within 2 hours; and
(iv) between 30% and 60% of the EE initially present in the composition is dissolved within 2 hours.

16. The method of claim 1, wherein after single-dose oral administration to the female subject, the oral contraceptive composition provides:
a Cₘₐₓ of EE ranging from 29.2 μg/mL to 36.9 μg/mL; and
a Cₘₐₓ of DNG ranging from 34.9 ng/mL to 48.5 ng/mL.

17. (canceled)

18. The method of claim 1, wherein after single-dose oral administration to the female subject, the oral contraceptive composition provides:
an AUC_{0-72 hr} of EE ranging from 435 pg×h/mL to 562 pg×h/mL; and
an AUC_{0-72 hr} of DNG ranging from 545 ng×h/mL to 689 ng×h/mL.

19. The method of claim 1, wherein after single-dose oral administration to the female subject, the oral contraceptive composition provides:
a Cₘₐₓ of EE ranging from 29.2 μg/mL to 36.9 μg/mL;
a Tmax of EE ranging from 3.1 hours to 4.7 hours;
an AUC_{0-72 hr} of EE ranging from 435 pg×h/mL to 562 pg×h/mL;
a Cₘₐₓ of DNG ranging from 34.9 ng/mL to 48.5 ng/mL;
a Tmax of DNG ranging from 3.0 hours to 4.4 hours; and
an AUC_{0-72 hr} of DNG ranging from 545 ng×h/mL to 689 ng×h/mL.

20. (canceled)

21. A method for oral contraception in a female subject in need thereof, the method comprising:
(i) a first phase wherein active daily dosage units of an oral contraceptive composition are administered to the female subject over a period of 21 to 27 days, with a proviso that no more than two of the 21 to 27 days may be non-consecutive; and
(ii) a second phase wherein no contraceptive composition is administered to the female subject over a period of 1 to 7 consecutive days,
wherein:
the oral contraceptive composition comprises 2 mg of 17α-cyanomethyl-17-β-hydroxyestra-4,9-dien-3-one (dienogest, DNG) and 0.02 mg of 17α-ethinylestradiol (ethynyl estradiol, EE) in an extended-release matrix;
the DNG and the EE are both subject to controlled release, such that after single-dose oral administration to the female subject, the oral contraceptive composition provides:
a Tmax of EE ranging from 3.1 hours to 4.7 hours, and
a Tmax of DNG ranging from 3.0 hours to 4.4 hours;
the extended-release matrix comprises a diluent, a polymeric matrix, a binder and a lubricant;
administration of one or two non-consecutive scheduled daily oral doses are missed, skipped or delayed for up to 24 hours from the scheduled daily administration.

22. The method of claim 21, wherein polymeric matrix comprises a hydroxypropyl methyl cellulose, and the diluent comprises a lactose monohydrate.

23. The method of claim 21, wherein the DNG and the EE are formulated such that, when the oral contraceptive composition is subjected to an in vitro dissolution test according to USP1 (basket) method using 500 mL of water at 37° C. (±0.5° C.) at a stirring rate of 75 rpm, the DNG and the EE, respectively, exhibit a dissolution profile such that:
(i) no more than 20% of the DNG initially present in the composition is dissolved within 0.5 hour;
(ii) no more than 25% of the EE initially present in the composition is dissolved within 0.5 hour;
(iii) between 30% and 60% of the DNG initially present in the composition is dissolved within 2 hours; and
(iv) between 30% and 60% of the EE initially present in the composition is dissolved within 2 hours.

24. The method of claim 21, wherein after single-dose oral administration to the female subject, the oral contraceptive composition provides:
a Cₘₐₓ of EE ranging from 29.2 μg/mL to 36.9 μg/mL;
a Tmax of EE ranging from 3.1 hours to 4.7 hours;
an AUC_{0-72 hr} of EE ranging from 435 pg×h/mL to 562 pg×h/mL;
a Cₘₐₓ of DNG ranging from 34.9 ng/mL to 48.5 ng/mL;
a Tmax of DNG ranging from 3.0 hours to 4.4 hours; and
an AUC_{0-72 hr} of DNG ranging from 545 ng×h/mL to 689 ng×h/mL.

25. The method of claim 1, wherein the DNG and the EE are provided in an extended-release matrix.

26. The method of claim 25, wherein the extended-release matrix is formed from a polymeric matrix forming agent.

27. The method of claim 25, wherein the extended-release matrix is formed from a granular core comprising a polymeric matrix forming agent and a diluent.

28. The method of claim 27, wherein a mass ratio of the polymeric matrix forming agent and the diluent in the granular core ranges from 2:1 to 10:1.

29. The method of claim 27, wherein the polymeric matrix forming agent comprises a hydroxypropyl methylcellulose, and the diluent comprises a lactose monohydrate.
